(19) United States
(12) Patent Application Publication
(10) Pub. No.: US 2024/0226002 A9
(43) Pub. Date: Jul. 11, 2024

(54) SOFT CHEWABLE FORMULATIONS WITH ACTIVE INGREDIENTS

(71) Applicant: Noble Pharma, LLC, Menomonie, WI (US)

(72) Inventors: Alok K. Kulshreshtha, Southlake, TX (US); Anita K. Sinha, Bloomington, IN (US); Daniel J. DuBourdieu, Limerick, ME (US)

(21) Appl. No.: 18/456,374

(22) Filed: Aug. 25, 2023

Related U.S. Application Data
(60) Provisional application No. 63/401,795, filed on Aug. 29, 2022.

(51) Int. Cl. A61K 9/00; A61K 31/365; A61K 31/403; A61K 31/439; A61K 31/4985; A61K 31/501; A61K 31/506; A61K 31/7048; A61K 47/36; A61K 47/46

(52) U.S. Cl. CPC A61K 9/0056; A61K 31/365; A61K 31/403; A61K 31/439; A61K 31/4985; A61K 31/501; A61K 31/506; A61K 31/7048; A61K 47/36; A61K 47/46

(57) ABSTRACT

The present disclosure generally relates to a soft chewable formulation, especially suitable for delivering active ingredients to animals and processes for preparation thereof. In some embodiments of the disclosure, a soft chewable veterinary formulation includes a starch, one or more carbohydrates, a flavoring agent, a lipid, and at least one active ingredients. In accordance with some embodiments, the starch in the soft chewable formulations includes a certain ratios of regular starch and pre-gelatinized starch.

### CROSS REFERENCE TO RELATED APPLICATIONS

This application claims the benefit of U.S. Provisional App. No. 63/401,795, filed on Aug. 29, 2022, which is hereby incorporated by reference in its entirety.

### FIELD OF THE DISCLOSURE

The present disclosure generally relates to the field of medicine and veterinary medicine. Generally, the disclosure relates to materials for delivering an active ingredient to animals.

### BACKGROUND OF THE DISCLOSURE

While drugs are important for maintaining the health of an individual, many drugs taste bad and are typically not palatable by themselves. This creates a problem to actually deliver the active drugs to individuals. As such, active drug ingredients are provided orally to humans and animals in many formats including as liquids, powders, capsules, tablets, and in soft chew formats, in attempts to mask the flavor and make the drugs more palatable to consume.

### SUMMARY OF THE DISCLOSURE

Disclosed herein are formulations useful for the treatment or amelioration of various diseases, disorders, or conditions.

Some embodiments pertain to a soft chewable veterinary formulation. In some embodiments, the formulation includes a starch between about 2% w/w and about 50% w/w, one or more carbohydrates between about 5% w/w and about 25% w/w, a flavoring agent between about 5% w/w and about 15% w/w, a disintegrant between about 0.1% w/w and about 10.0% w/w, a lipid between about 5% w/w and about 15% w/w, and at least one active ingredient between about 0.01% w/w and about 20% w/w, wherein, the starch comprises a regular starch and a pre-gelatinized starch that is in a ratio from about 3:1 to about 15:1. In some embodiments, the flavoring agent is an animal-derived flavoring agent. In some embodiments, the animal-derived flavoring agent is chicken liver powder, pork liver powder, beef liver powder, ham, fish, or a combination thereof. In some embodiments, the chicken-liver comprises about 8% to about 14% of the soft chewable veterinary formulation. In some embodiments, the soft chewable veterinary formulation further comprises a humectant. In some embodiments, the humectant is glycerin, glycerol triacetate, polydextrose, lactic acid, or a combination thereof. In some embodiments, the humectant comprises between about 8% w/w and about 20% w/w of the soft chewable veterinary formulation. In some embodiments, the soft chewable veterinary formulation further comprises a preservative. In some embodiments, the preservative is benzalkonium chloride, benzethonium chloride, benzoic acid, benzyl alcohol, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, propyl paraben, myristyl gamma-picolinium chloride, methylparaben, propylparaben, quaternary ammonium compounds, or combinations thereof. In some embodiments, the preservative comprises between about 0.05% w/w to about 3.0% w/w of the soft chewable veterinary formulation. In some embodiments, the soft chewable veterinary formulation further comprises a binder. In some embodiments, the binder comprises between about 0.1% to about 5.0% w/w. In some embodiments, the disintegrant is sodium starch glycolate, crospovidone, croscarmellose sodium, microcrystalline cellulose, alginic acid, veegum, bentonite, croscarmellose, or combinations thereof. In some embodiments, the disintegrant comprises between about 0.1% w/w to about 5.0% w/w of the soft chewable veterinary formulation. In some embodiments, the soft chewable veterinary formulation further comprises an emulsifying agent. In some embodiments, the emulsifying agent is potassium laurate, triethanolamine stearate, sodium lauryl sulfate, alkyl polyoxyethylene sulfates, sodium dodecyl sulfate, dioctyl sodium sulfosuccinate, polyoxyethylene fatty acid derivatives of the sorbitan esters, polyoxyethylene fatty alcohol ethers sorbitan fatty acid esters polyoxyethylene alkyl ethers polyoxyethylene sorbitan fatty acid esters, polyoxyethylene polyoxypropylene block copolymers, polyethylene glycol 400 monostearate, lanolin alcohols, hydrophilic colloids, alginates, acacia, tragacanth, xanthan, vitamin E, pectin, gelatin, casein, lecithin, or combinations thereof. In some embodiments, the emulsifying agent is less than about 5.0% w/w of the soft chewable veterinary formulation. In some embodiments, the at least one active ingredient is a moisture-sensitive active ingredient. In some embodiments, the at least one active ingredient is at least one active anti-parasitic active ingredient. In some embodiments, the at least one active ingredient is selected from the group consisting of pyrantel pamoate, ivermectin, pimobendan, firocoxib, carporfen, maropitant citrate, and praziquantel. In some embodiments, the lipid is a vegetable oil. In some embodiments, the vegetable oil is refined soybean oil. In some embodiments, the one or more carbohydrates is a simple carbohydrate. In some embodiments, the simple carbohydrate is dextrose, ribose, fructose, sucrose, maltose, erythritol, xylitol, hydrogenated isomaltulose, maltitol, or combinations thereof. In some embodiments, the soft chewable veterinary formulation further comprises a sweetener. In some embodiments, the sweetener is honey, maple syrup, corn syrup, synthetic sweeteners, natural sweeteners, or combinations thereof. In some embodiments, the sweetener is between about 1% to about 10% of the soft chewable veterinary formulation. In some embodiments, the soft chewable veterinary formulation further comprises a coloring agent. In some embodiments, the coloring agent is caramel color liquid. In some embodiments, the coloring agent is between about 0.1% to about 4.0% of the soft chewable veterinary formulation. In some embodiments, the chewable veterinary formulation further comprises one or more components selected from the group consisting of surfactants, wetting agents, pH stabilizers, a softening agent, and a solvent. In some embodiments, the soft chewable veterinary formulation has a water content of less than 5% w/w. In some embodiments, the chewable veterinary formulation has an added water content of less than 1%. In some embodiments, the at least one active ingredient is a nutrient. In some embodiments, the soft chewable veterinary formulation further comprises a nutrient.

In some embodiments, the soft chewable veterinary formulation includes a liquid between 20% to about 40%, a regular starch between 20% to about 50%, a pre-gelatinized starch between 2% to about 50%, and an active ingredient between 0.01% and 15%, wherein, the total of the active ingredient and the regular starch comprises 35% to 50% of the soft chewable veterinary formulation, wherein, the ratio of the active ingredient and the regular starch to the pre-gelatinized starch is in a ratio from about 3:1 to about 15:1.

Some embodiments pertain to a method of treating, controlling, preventing, or ameliorating a disease or condition in a subject in need thereof. In some embodiments, the method includes administering to the subject a soft chewable formulation as described herein thereby, treating, controlling, preventing, or ameliorating the disease or condition in the subject in need thereof. In some embodiments, the disease or condition is associated with a viral infection. In some embodiments, viral infection is selected from canine distemper, canine influenza, canine parvovirus, herpes, rabies, feline calicivirus, coronavirus, feline leukemia virus, feline panleukopenia virus, feline calicivirus, feline herpes virus, varicella zoster virus, herpes simplex virus, cytomegalovirus, or Epstein-Barr virus. In some embodiments, the disease or condition is associated with a bacterial infection or condition. In some embodiments, the bacterial infection or condition is selected from Bacillus anthracia, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumonia, Chlamydia trachomatis, Chlamydophila psittaci, Clostridium botulinum, Clostridium difficile, Clostridium perfringens, Clostridium tetani, Corynebacterium diphtheria, Enterococcus faecalis, Enterococcus faecium, Escherichia coli, Francisella tularensis, Haemophilus influenza, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumonia, Neisseria gonorrhoeae, Neisseria meningitides, Pseudomonas aeruginosa, Rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumonia, Streptococcus pyogenes, Treponema pallidum, Ureaplasma urealyticum, Vibrio cholera, Yersinia pestis, Yersinia enterocolitica, Yersinia pseudotuberculosis. In some embodiments, the disease or condition is associated with a fungus disease or condition. In some embodiments, the fungal disease or condition is selected from Histoplasma capsulatum (causes Histoplasmosis), Blastomyces dermatitidis (causes Blastomycosis), and Coccidioides immitis (causes Coccidioidomycosis (Valley Fever)), Aspergillosis fumigaus (causes Aspergillosis), Aspergillus tereus (causes Disseminated Aspergillosis), Aspergillus deflectus, Aspergillus niger, Candida albicans (causes Candidiasis), Cryptococcus neoformans (causes Cryptococcosis), Cryptococcus gattii, Geotrichum candidum (causes Geotrichosis), Pythium insidiosum (causes Oomycosis also called pythiosis), Rhinosporidium seeberi (causes Rhinosporidiosis), and Sporothrix schenckii (causes Sporotrichosis).

Some embodiments pertain to a method of treating, controlling, preventing, or ameliorating a parasitic, insect, acarid, or helminth infestation in a subject in need thereof. In some embodiments, the method includes administering to the subject a soft chewable formulation as described herein, thereby, treating, controlling, preventing, or ameliorating the parasitic, insect, acarid, or helminth infestation in a subject in need thereof.

Some embodiments pertain to a method of treating, controlling, preventing, or ameliorating an infection in a subject in need thereof. In some embodiments, the method includes administering to the subject a soft chewable formulation as described herein, thereby, treating, controlling, preventing, or ameliorating the infection in a subject in need thereof. In some embodiments, treating the infection reduces infection by at least about 90% after about 5 days. In some embodiments, the soft chewable formulation is administered to the subject at least once a day. In some embodiments, the soft chewable formulation is administered to the subject once a week. In some embodiments, the soft chewable formulation is administered to the subject once a month. In some embodiments, the soft chewable formulation is administered to the subject once every 3 months. In some embodiments, the subject is a companion animal. In some embodiments, the companion animal is a cat, dog, or horse.

Some embodiments relate to a method of treating, controlling, preventing, managing, or ameliorating pain in a subject in need thereof. In some embodiments, the method includes administering to the subject a soft chewable formulation as described herein, thereby, treating, controlling, preventing, or ameliorating the pain in a subject in need thereof.

Some embodiments relate to a method of treating, controlling, preventing, managing, or ameliorating heart disease in a subject in need thereof. In some embodiments, the method includes administering to the subject a soft chewable formulation as described herein, thereby treating, controlling, preventing, or ameliorating the chronic heart disease in a subject in need thereof.

Some embodiments relate to a method of treating, controlling, preventing, managing, or ameliorating nausea and vomiting in a subject in need thereof. In some embodiments, the method includes administering to the subject a soft chewable formulation as described herein, thereby, treating, controlling, preventing, or ameliorating the nausea and vomiting in a subject in need thereof.

### BRIEF DESCRIPTION OF THE DRAWINGS

FIG. 1 illustrates a line graph depicting a dissolution profile for firocoxib soft chewables over time.

FIG. 2 is a picture of a soft chewable of an embodiment of the disclosure.

FIG. 3 is a picture of a side perspective of a soft chewable of an embodiment of the disclosure.

FIG. 4 is a picture illustrating a crushed soft chewable of an embodiment of the disclosure.

FIG. 5 is a picture illustrating a crushed soft chewable of an embodiment of the disclosure.

### DETAILED DESCRIPTION OF DISCLOSURE

Soft chew formats are intended to provide an active ingredient in a palatable form that can be orally consumed. The parameters that ideally should be met during manufacturing include keeping the active ingredients stable over a defined period of time while maintaining the soft chew matrix in a manner that allows the similar degree of softness, texture, dissolution parameters and palatability that was initially found at the time of manufacturing.

Some soft chewable formulations present stability issues of the actives and the matrix itself for maintaining palatability, softness, texture, and dissolution parameters over the defined shelf life. This is believed to be due to the interaction of drugs and specific ingredients and concentrations of these ingredients in the soft chew matrix. New FDA regulations require that the dissolution of the drug product be tested using a discriminating in vitro dissolution method. Hence, a new soft chewable formulation that can meet the regulatory agencies' requirements for dissolution in physiologically relevant media is needed. This has been a challenge for a number of drug actives in soft chew formulations. The ability to extrude any particular formulation that is not too dry or too sticky for the equipment used continues to be a challenge to manufacturers. Furthermore, maintaining the stability of many active drugs is a challenge in soft chew formats because the presence of water is typically detrimental to drug actives.

In some embodiments, there is provided a palatable soft chew oral dosage format for administering pharmaceuticals or nutraceuticals to animals and humans that could avoid some of the challenges of existing soft chews. In some embodiments, the soft chewable formulation described herein provides for a formulation that before mastication is not sticky or powdery. In some embodiments, the formulation comprises a matrix with a desired firmness. In some embodiments, the soft chewable formulation may provide relatively fast disintegration. An advantage of this embodiment may be that due to the relatively fast disintegration, the mastication of the formulation described herein begins with a relatively solid matrix that after initial pressure is applied to it transforms into a soft, grainy amalgam. In some embodiments, the soft chewable formulation meets FDA requirements for dissolution in physiologically relevant media. In some embodiments, the soft chewable formulation includes a low water content and remains suitable for moisture-sensitive active ingredients. In other aspects of the disclosure, processes for preparing a soft chewable formulation are provided. In some embodiments, the soft chewable formulation may remain stable for the intended shelf life. In some embodiments, the soft chewable formulation may remain stable for the intended degree of desired dissolution. In some embodiments, the soft chewable formulation may be highly palatable for veterinary animals.

The section headings used herein are for organizational purposes only and are not to be construed as limiting the subject matter described. Features disclosed under one heading (such as a composition) can be used in combination with features disclosed under a different heading (a method of treating). Unless defined otherwise, all technical and scientific terms used herein have the same meaning as is commonly understood by one of ordinary skill in the art. It should be noted that the use of particular terminology when describing certain features or aspects of the disclosure should not be taken to imply that the terminology is being re-defined herein to be restricted to include any specific characteristics of the features or aspects of the disclosure with which that terminology is associated.

While the disclosure has been illustrated and described in detail in the foregoing description, such description is to be considered illustrative or exemplary and not restrictive. The disclosure is not limited to the disclosed embodiments. Variations to the disclosed embodiments can be understood and effected by those skilled in the art in practicing the claimed disclosure, from a study of the disclosure and the appended claims.

### Definitions

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function of the disclosure, but instead as merely intended to highlight alternative or additional features that may or may not be utilized in a particular embodiment of the disclosure. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

As used in the specification and the appended claims, the singular forms “a,” “an” and “the” include plural referents unless the context clearly dictates otherwise. The use of “or” or “and” means “and/or” unless stated otherwise. Furthermore, use of the term “including” as well as other forms, such as “include”, “includes,” and “included,” is not limiting. The mere fact that certain measures are recited in mutually different dependent claims does not indicate that a combination of these measures cannot be used to advantage.

The term “about” or “approximately” means within an acceptable error range for the particular value as determined by one of ordinary skill in the art, which will depend in part on how the value is measured or determined, e.g., the limitations of the measurement system. Where particular values are described in the application and claims, unless otherwise stated, the term “about” means “within an acceptable error range for the particular value.”

The term “animal” is used herein to include all vertebrate animals, including transgenic animals. It also includes an individual animal in all stages of development, including embryonic and fetal stages. As used herein, the term “production animals” is used interchangeably with “livestock animals” and refers generally to animals raised primarily for food. For example, such animals include, but are not limited to, cattle (bovine), sheep (ovine), pigs (porcine or swine), poultry (avian), and the like. As used herein, the term “cow” or “cattle” is used generally to refer to an animal of bovine origin of any age. Interchangeable terms include “bovine”, “calf”, “steer”, “bull”, “heifer”, “cow” and the like. As used herein, the term “pig” is used generally to refer to an animal of porcine origin of any age. Interchangeable terms include “piglet”, “sow” and the like. As used herein, the term “companion animals” is used herein to refers to a domestic animal. Companion animal is used generally to refer to a domestic dog, cat, rabbit, ferret, horse, or the like.

The term “subject” includes animals.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C1-C7 alkylamine, cyclohexylamine, triethanolamine, ethylenediamine, and salts with amino acids such as arginine and lysine. It is understood that the methods and formulations described herein include the use of pharmaceutically acceptable salts and/or conformers of compounds of disclosed embodiments, as well as metabolites and active metabolites of these compounds having the same type of activity. A conformer is a structure that is a conformational isomer. Conformational isomerism is the phenomenon of molecules with the same structural formula but different conformations (conformers) of atoms about a rotating bond. Likewise, it is understood that the compounds described herein, include the compound in any of the forms described herein (e.g., pharmaceutically acceptable salts, polymorphs, enantiomeric forms, tautomeric forms, and the like).

The terms “effective amount” and “therapeutically effective amount” are broad terms, and are to be given their ordinary and customary meaning to a person of ordinary skill in the art (and are not to be limited to a special or customized meaning), and refer without limitation to a sufficient amount of an agent or a compound being administered which will relieve to some extent one or more of the symptoms of the disease and/or condition being treated. In some embodiments, the result can be reduction and/or alleviation of the signs, symptoms, or causes of a disease, or any other desired alteration of a biological system. For example, an “effective amount” for therapeutic uses is an amount of the composition comprising a compound as disclosed herein required to provide a clinically significant decrease in disease symptoms. An appropriate “effective” amount in any individual case may be determined using techniques, such as a dose escalation study. Where a drug has been approved by the U.S. Food and Drug Administration (FDA) or a counterpart foreign medicines agency, a “therapeutically effective amount” optionally refers to the dosage approved by the FDA or its counterpart foreign agency for treatment of the identified disease or condition.

The term “bioavailability” includes, generally, the degree to which a drug or other substance (e.g., testosterone) becomes available to a subject following ingestion, administration, or exposure. In one embodiment, for example, the bioavailability of one or more of the compounds disclosed herein may be the bioavailability to a particular target tissue (e.g., the liver). For example, in an embodiment, the particular target tissue may require traversal of the stomach, the small intestines, and/or the blood brain barrier, therefore the bioavailability data may be obtained from this particular target tissue.

The terms “treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

The term “co-administration” and similar terms as used herein are broad terms, and are to be given their ordinary and customary meaning to a person of ordinary skill in the art (and are not to be limited to a special or customized meaning), and refer without limitation to administration of the selected therapeutic agents to a single patient, and are intended to include treatment regimens in which the agents are administered by the same or different route of administration or at the same or different time. In some embodiments, the compounds disclosed herein are co-administered.

Additionally, the phrase “consisting essentially of” will be understood to include those elements specifically recited and those additional elements that do not materially affect the basic and novel characteristics of the claimed technology. The phrase “consisting of” excludes any element not specified. The transitional phrase “consisting of” excludes any element, step, or ingredient not specified in the claim.

The term “pharmaceutical composition” refers to a mixture of one or more compounds disclosed herein with other chemical components, such as diluents or carriers. The pharmaceutical composition facilitates administration of the compound to an organism. Pharmaceutical compositions will generally be tailored to the specific intended route of administration.

As used herein, a “carrier” refers to a compound that facilitates the delivery of a compound into cells or tissues.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration.

As used herein, the term “disintegrant” includes conventional disintegrants and other disintegrants known in the art as super-disintegrants.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

As used herein, the term “stability” or “stabilization” refers to the stability of the pharmaceutical composition in total and in particular to the stability of the active ingredient (i.e. the an active ingredient) or nutrient itself, specifically during formulation, filling, shipment, storage and administration.

As used herein, the term “regular starch” refers to an untreated or naturally occurring polymeric carbohydrate or a synthesized compound with substantially the same properties. An example of a regular starch can include an untreated, native starch extracted from a plant, such as corn, potato, or rice. Unlike modified starches, such as pregelatinized starches, which may undergo one or more chemical or physical treatments to alter their native properties, regular starch has properties that are the same as or functionally equivalent to those of starches in their natural, substantially unmodified forms as extracted from plants.

As used herein, the term “pregelatinized starch” or “pre-gelatinized starch” refers to a chemically or mechanically modified form of a native starch. Pregelatinized starches may undergo a process that disrupts the hydrogen bonds between starch molecules, causing the crystalline structure to change into an amorphous state. In some embodiments, the starch is then rapidly dried, locking it into a state that allows for cold-water solubility and instant thickening upon rehydration. This modification may enhance various of the starch's functional properties, making it more stable under various conditions such as heat, acidity, and shear stress. Pregelatinized starches can include a starch which yield a dispersion, paste or gel when dispersed in cold water. Pregelatinized starches may include a starch that has been modified and then dried, usually in a process involving heat and/or changes in moisture. For example, this treatment can induce the starch to instantly thicken upon the addition of cold or warm water.

As used herein, the term “weight percent,” when referring to a component, is the weight of the component divided by the weight of the composition that includes the component, multiplied by 100%. For example, the weight percent of component A when 5 grams of component A is added to 95 grams of component B is 5% (e.g., 5 g A/(5 g A+95 g B)×100%).

### Formulations

In aspects, a soft chewable formulation is provided herein including at least one active ingredient. In some embodiments, the at least one active ingredient includes one active ingredient. In some embodiments, the at least one active ingredient includes two active ingredients. In some embodiments, the at least one active ingredient includes three active ingredients. In some embodiments, the soft chewable formulation is formulated as a soft chewable veterinary formulation.

In some embodiments, the at least one active ingredient may include agents that are, for example, anticonvulsant, anticoagulant, antifungal, antiparasitic (endo- or ecto-), antipyretic, antiseptic, acaricidic, anthelmintic, beta-blockers, bisphosphonates, bronchodilator, chelating agents, chemotherapy agents, congestive heart failure, insecticidal, antiemetic, antimicrobial, antiviral, antibiotic, anti-inflammatory, hormone, hormone modulator, laxatives, muscle relaxant, statins, stimulant, vasodilator, psychotropic, pain management, and proton pump inhibitors.

In some embodiments, the at least one active ingredient is an insect growth regulator. In some embodiments, the insect growth regulator includes, for a non-limiting example, isooxazoline compounds such as azadirachtin, afoxolaner, fluralaner, sarolaner, benzoylphenylureas such as diflubenzuron, clofentezine, chlorfluazuron, etoxazole, diflubenzuron, difenolan, flufenoxuron, flucycloxuron, s-methoprene, nitenpyram, novaluron, noviflumuron, oxamyl, hexaflumuron, hexythiazox, hydroprene, propargite, spiromesifen, spirotetramat, tolfenpyrad, triflumuron, teflubenzuron, fenoxycarb or substances like fenoxycarb, pyridalyl, pyriproxifen, methoprene, kinoprene, bydroprene, cyromazine, buprofezin, pymetrozine and combinations or derivatives thereof in free form or in the form of a pharmaceutical acceptable salt.

In some embodiments, the at least one active ingredient is an anthelmintics comprising a broad spectrum macrocyclic lactones, such as avermectins, milbemycins and derivatives thereof, including, but not limited to, ivermectin, doramectin, moxidectin, selamectin, emamectin, eprinomectin, milbemectin, abamectin, milbemycin oxime, nemadectin, Spinosad, derquantel, oxfendazole, monepantel, and combinations or derivatives thereof, in free form or in the form of a pharmaceutical acceptable salt. In some embodiments, the at least one active ingredient comprises a broad spectrum macrocyclic lactone and at least one of a fipronil, Spinosad, paraherquamide, derquantel, oxfendazole, monepantel, or praziquantel. In some embodiments, the at least one active ingredient comprises a combination of ivermectin and praziquantel.

In some embodiments, the at least one active ingredient is at least one active anti-parasitic active ingredient. In some embodiments, the anti-parasitic active ingredient is a benzimidazoles, isoquinoline-pryazine derivative, amino-acetonitrile derivative, pyridine, metronidazole, amprolium, emodepside, toltrazuril, fipronil, melarsomine dihydrochloride, diclazuril, and combinations or derivatives thereof in free form or in the form of a pharmaceutical acceptable salt. In some embodiments, the benzimidazole is fenbendazole or albendazole. In some embodiments, the iosquinoline-pyrazine derivative is praziquantel. In some embodiments, the amino-acetonitrile derivative is monepantel. In some embodiments, the pyridine is nitenpyram. In some embodiments, the at least one active ingredient is pyrantel pamoate.

In some embodiments, at least one active ingredient is at least one anti-inflammation agent. In some embodiments, the at least anti-inflammation agent is a non-steroidal anti-inflammatory agent (NSAIDs). In some embodiments, the NSAIDs may be selected from, but is not limited to, carprofen, deracoxib, firocoxib, meloxicam, robenacoxib, flunixin meglumine, ketoprofen, tepoxalin, piroxicam, phenylbutazone, aspirin and combinations or derivatives thereof in free form or in the form of a pharmaceutically acceptable salt. In some embodiments, the at least anti-inflammation agent is a corticosteroid. In some embodiments, the corticosteroid may be selected from, but is not limited to, prednisone, prednisolone, dexamethasone, triamcinolone, hydrocortisone, methylprednisolone, fludrocortisone, and combinations or derivatives thereof in free form or in the form of a pharmaceutical acceptable salt. In some embodiments, the at least one anti-inflammation agent may be selected from pentosan polysulfate sodium. In some embodiments, the at least one anti-inflammation agent may be selected from adequan (polysulfated glycosaminoglycan).

In some embodiments, the at least one active ingredient is at least one vasodilator. In some embodiments, the at least one vasodilator includes, but is not limited to, an Angiotensin-Converting Enzyme (ACE) Inhibitor, calcium channel blocker, nitrate vasodilators, direct vasodilators, phosphodiesterase inhibitors, theophylline, pentoxifylline and combinations or derivatives thereof in free form or in the form of a pharmaceutical acceptable salt. In some embodiments, the ACE inhibitor may be selected from enalapril, benazepril, or ramipril. In some embodiments, the calcium channel blockers may be selected from amlodipine besylate or diltiazem. In some embodiments, the nitrate vasodilators may be selected from nitroglycerin or isosorbide dinitrate. In some embodiments, the direct vasodilators may be selected from hydralazine, minoxidil, or prazosin. In some embodiments, the phosphodiesterase inhibitors may be selected from sildenafil or tadalafil.

In some embodiments, the at least one active ingredient is at least one antiemetic. In some embodiments, the at least one antiemetic may be selected from, but not limited to, serotonin (5-HT3) receptor antagonists, neurokinin receptor antagonists, phenothiazines, dopamine antagonists, H1 antihistamines, substance P antagonists, mirtazapine, maropitant citrate and combinations or derivatives thereof in free form or in the form of a pharmaceutical acceptable salt. In some embodiments, the serotonin (5-HT3) receptor antagonist is ondansetron. In some embodiments, the neurokinin receptor antagonist is maropitant citrate. In some embodiments, the phenothiazines may be selected from chlorpromazine or prochlorperazine. In some embodiments, the dopamine antagonists may be selected from metoclopramide or domperidone. In some embodiments, the H1 antihistamines may be selected from diphenhydramine (Benadryl) or dimenhydrinate (Dramamine).

In some embodiments, the at least one active ingredient is at least one sympathomimetic agent. In some embodiments, the at least one sympathomimetic agent may be selected from, but not limited to, alpha and beta agonists, beta-2 adrenergic agonists, alpha-2 adrenergic agonists, mixed alpha and beta agonists, indirect sympathomimetics, dobutamine and combinations or derivatives thereof in free form or in the form of a pharmaceutical acceptable salt. In some embodiments, the alpha and beta agonists may be selected from epinephrine, norepinephrine, isoproterenol, or dopamine. In some embodiments, the beta-2 adrenergic agonists may be selected from albuterol, terbutaline, or clenbuterol. In some embodiments, the alpha-2 adrenergic agonists may be selected from xylazine, dexmedetomidine, detomidine, or romifidine. In some embodiments, the indirect sympathomimetics may be selected from amphetamines or phenylpropanolamine.

In some embodiments, the at least one active ingredient is at least one drug or active agent for pain management. For example, a drug or active agent may inhibit COX-1 and COX-2 and may be a NSAID. In some embodiments, the at least one drug or active agent for pain management may include, but are not limited to, NSAIDs, opioids, alpha-2 adrenergic agonists, local anesthetics, adjunctive analgesics, maropitant citrate, acetaminophen, paracetamol and combinations or derivatives thereof in free form or in the form of a pharmaceutical acceptable salt. In some embodiments, the NSAIDs for pain management may be selected from carprofen, firocoxib, meloxicam, deracoxib, robenacoxib, ketoprofen, flunixin meglumine, or phenylbutazone. In some embodiments, the opioids are selected from morphine, hydromorphone, buprenorphine, fentanyl, tramadol, butorphanol, methadone, or oxymorphone. In some embodiments, the alpha-2 adrenergic agonists may be selected from dexmedetomidine or xylazine. In some embodiments, the local anesthetic may be lidocaine, bupivacaine, or mepivacaine. In some embodiments, the adjunctive analgesics may be selected from gabapentin, amitriptyline, tricyclic antidepressants, or NMDA antagonists (for example, ketamine).

In some embodiments, the soft chewable formulation includes one or more active ingredients may be selected from, but is not limited to, omeprazole, famotidine, fluconazole, itraconzole, ketoconazole, levetiracetam, zonisamide, furosemide, pimobendane, levothyroxine, methimazole, amoxicillin/clavulanate, cephalexin, doxycycline, metronidazole, enrofloxacin, cyclosporine, ketoconazole, gabapentin, tramadol, oxytetracycline, tilmicosin, sulfadimethoxine, levamisole, bismuth subsalicylate, prostaglandins, hormones, ranitidine, sucralfate, psyllium, methocarbamol, trilostane, guafenisen, pentobarbital, phenobarbital, mirtazapine, misoprostol, omeperazole, cisapride, febantel, spironolactone, atropine, or a combination thereof.

In some embodiments, the soft chewable formulation includes about 0.001% to about 40% w/w of at least one active ingredient. In some embodiments, the soft chewable formulation includes at least about: 0.001%, 0.005%, 0.01%, 0.05%, 0.1%, 0.5%, 1%, 1.5%, 2.0%, 2.5%, 3.0%, 3.5%. 4.0%, 4.5% 5.0%, 6.0%, 7.0%, 8.0%, 8.5%, 9.0%, 9.5% 10%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 22.5%, 25%, 27.5%, 30.0%, 32.5%, 35.0%, 37.5%, 40.0% w/w of at least one active ingredient, or ranges including and/or spanning the aforementioned values. In some embodiments, the soft chewable formulation includes about 0.001% w/w to about 6.0% w/w of at least one active ingredient. In some embodiments, the soft chewable formulation includes about 0.01% w/w to about 5.0% w/w of at least one active ingredient. In some embodiments, the soft chewable formulation includes about 0.05% w/w to about 4.0% w/w of at least one active ingredient. In some embodiments, the soft chewable formulation includes about 1.0% w/w to about 6.0% w/w of at least one active ingredient. In some embodiments, the soft chewable formulation includes about 0.01% w/w to about 1.0% w/w of at least one active ingredient. In some embodiments, the soft chewable formulation includes about 2.0% w/w to about 5.0% w/w of at least one active ingredient. In some embodiments, the soft chewable formulation includes about 0.01% w/w of at least one active ingredient. In some embodiments, the soft chewable formulation includes about 0.6% w/w of at least one active ingredient. In some embodiments, the soft chewable formulation includes about 0.85% w/w of at least one active ingredient. In some embodiments, the soft chewable formulation includes about 0.1% w/w of at least one active ingredient. In some embodiments, the soft chewable formulation includes about 1.4% w/w of at least one active ingredient. In some embodiments, the soft chewable formulation includes about 2.25% w/w of at least one active ingredient. In some embodiments, the soft chewable formulation includes about 2.5% w/w of at least one active ingredient. In some embodiments, the soft chewable formulation includes about 2.85% w/w of at least one active ingredient. In some embodiments, the soft chewable formulation includes about 4.0% w/w of at least one active ingredient. In some embodiments, the soft chewable formulation includes about 4.1% w/w of at least one active ingredient. In some embodiments, the soft chewable formulation includes about 5.0% w/w of at least one active ingredient. In some embodiments, the soft chewable formulation includes about 5.7% w/w of at least one active ingredient. In some embodiments, the soft chewable formulation includes about 8.2% w/w of at least one active ingredient. In some embodiments, the soft chewable formulation includes about 16.4% w/w of at least one active ingredient. In some embodiments, the at least one active ingredient is ivermectin. In some embodiments, the at least one active ingredient is pyrantel pamoate. In some embodiments, the at least one active ingredient is firocoxib. In some embodiments, the at least one active ingredient is carprofen. In some embodiments, the at least one active ingredient is maropitant citrate. In some embodiments, the at least one active ingredient is pimobendan. In some embodiments, the at least one active ingredient is praziquantel. In some embodiments, the at least one active agent is a combination of ivermectin and pyrantel pamoate.

In some embodiments, the amount of active ingredient in the soft chewable formulation may also be specified, as is typical in the art, in terms of the weight of the active ingredient per dosage form. In some embodiments, the soft chewable formulation contains at least about 1 mg, at least about 2.5 mg, at least about 5 mg, at least about 7.5 mg, at least about 10 mg, at least about 15 mg, at least about 20 mg, at least about 25 mg, at least about 30 mg, at least about 40 mg, at least about 50 mg, at least about 75 mg, at least about 100 mg, at least about 150 mg, at least about 200 mg, at least about 250 mg of active ingredients, or ranges including and/or spanning the aforementioned values. In some embodiments, the soft chewable formulation includes from about 5 mg to about 2000 mg, from about 10 mg to about 1500 mg, from about 10 mg to about 1000 mg, from about 10 mg to about 500 mg, from about 20 mg to about 2000 mg, from about 20 mg to about 1500 mg, from about 20 mg to about 1000 mg, from about 20 mg to about 500 mg, from about 50 mg to about 2000 mg, from about 50 mg to about 1500 mg, from about 50 mg to about 1000 mg, or from about 50 mg to about 500 mg of at least one active ingredient. In some embodiments, the soft chewable formulation includes at least one active ingredient at a dose from at least 0.005 mg/kg, 0.075 mg/kg, 0.01 mg/kg, 0.015 mg/kg, 0.02 mg/kg, 0.025 mg/kg, 0.05 mg/kg, 0.075 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg, 2 mg/kg, 2.5 mg/kg, 5 mg/kg, 7.5 mg/kg, 10 mg/kg or ranges including and/or spanning the aforementioned values.

In some embodiments, the soft chewable formulation comprises one or more carbohydrates. In some embodiments, the one or more carbohydrates is a simple carbohydrate. In some embodiments, the simple sugar is a monosaccharide. In some embodiments, the simple sugar is a combination of monosaccharides. In some embodiments, the simple sugar is a disaccharide. In some embodiments, the simple sugar is a combination of monosaccharides and disaccharides. Examples of simple carbohydrate includes, but are not limited to, dextrose, ribose, glucose, galactose, lactose, mannose, sorbitol, isomalt, trehalose, mannitol, lactitol, glyceradehyde, sorbose, fructose, sucrose, maltose, erythritol, xylitol, hydrogenated isomaltulose, maltitol, or combinations thereof. In some embodiments, the simple carbohydrate is dextrose. In some embodiments, the dextrose is dextrose monohydrate. In some embodiments, the dextrose is dextrose anhydrous. In some embodiments, the dextrose is a combination of dextrose monohydrate and dextrose anhydrous. In some embodiments, the simple carbohydrate is glucose. In some embodiments, the simple carbohydrate is galactose. In some embodiments, the simple sugar is mannose.

In some embodiments, the soft chewable formulation includes about 5% to about 25% w/w of a simple carbohydrate. In some embodiments, the soft chewable formulation includes about 5.0%, 7.5%, 10%, 12.5%, 15%, 17.5%, 20%, 22.5%, 25%, w/w of a simple carbohydrate, or ranges including and/or spanning the aforementioned values. In some embodiments, the soft chewable formulation includes about 5.0% w/w to about 15.0% w/w of a simple carbohydrate. In some embodiments, the soft chewable formulation includes about 7.0% w/w to about 14.0% w/w of a simple carbohydrate. In some embodiments, the soft chewable formulation includes about 10.0% w/w to about 15.0% w/w of a simple carbohydrate. In some embodiments, the soft chewable formulation includes about 11.0% w/w to about 14.0% w/w of a simple carbohydrate. In some embodiments, the soft chewable formulation includes about 5.0% w/w to about 10.0% w/w of a simple carbohydrate. In some embodiments, the soft chewable formulation includes about 7.0% w/w to about 11.0% w/w of a simple carbohydrate. In some embodiments, the soft chewable formulation includes about 10.0% w/w to about 25.0% w/w of a simple carbohydrate. In some embodiments, the soft chewable formulation includes about 7.5% w/w of a simple carbohydrate. In some embodiments, the soft chewable formulation includes about 9% w/w of a simple carbohydrate. In some embodiments, the soft chewable formulation includes about 9.5% w/w of a simple carbohydrate. In some embodiments, the soft chewable formulation includes about 10% w/w of a simple carbohydrate. In some embodiments, the soft chewable formulation includes about 11% w/w of a simple carbohydrate. In some embodiments, the soft chewable formulation includes about 12% w/w of a simple carbohydrate. In some embodiments, the soft chewable formulation includes about 13% w/w of a simple carbohydrate. In some embodiments, the soft chewable formulation includes about 14% w/w of a simple carbohydrate. In some embodiments, the soft chewable formulation includes about 15% w/w of a simple carbohydrate. In some embodiments, the soft chewable formulation includes about 22% w/w of a simple carbohydrate. In some embodiments, the soft chewable formulation includes about 23% w/w of a simple carbohydrate. In some embodiments, the soft chewable formulation includes about 24% w/w of a simple carbohydrate.

In some embodiments, the one or more carbohydrates is a polymeric carbohydrate. Non-limiting examples of a polymeric carbohydrate include agar, alginate, amylose, amylopectin, arabinogalactan, beta-glucans, carrageenan, cellulose, fucoidan, gellan gum, gum Arabic, hemicellulose, inulin, konjac glucomannan, laminarin, mannan, microcrystalline cellulose, psyllium husk, starch, guar gum, glycogen, cyclodextrin, hyaluronic acid, chitin, xanthan, chitosan, pectin, pullulan, and combinations thereof. In some embodiments, the polymeric carbohydrate is a starch. In some embodiments, the starch is modified starch. In some embodiments, the starch is a regular starch. In some embodiments, the starch is a pregelatinized starch. In some embodiments, the starch is a partially gelatinized starch. In some embodiments, the starch is a gelatinized starch. In some embodiments, the starch is a hydrogenated starch hydrolysate. In some embodiments, the starch is a sodium starch glycolate. In some embodiments, the one or more carbohydrates may be selected from the group selected from arrowroot starch, barley starch, buckwheat starch, cassava starch, green banana starch, tapioca starch, maize starch, millet starch, mung bean starch, corn starch, oat starch, pea starch, potato starch, rice starch, rye starch, sago starch, sorghum starch, sweet potato starch, water chestnut starch, wheat starch, yam starch, and combinations thereof.

In some embodiments, the soft chewable formulation includes about 2% to about 50% w/w of a polymeric carbohydrate. In some embodiments, the polymeric carbohydrate includes one or more starches. In some embodiments, the soft chewable formulation includes about 2.0%, 4.0%, 6.0%, 8.0%, 10.0%, 12.0%, 14.0%, 16.0%, 18.0%, 20.0%, 22.0%, 24.0%, 26.0%, 28.0%, 30.0%, 32.0%, 34.0%, 36.0%, 38.0%, 40.0%, 42.0%, 44.0%, 46.0%, 48.0%, 50.0%, 55.0% w/w of a polymeric carbohydrate, or ranges including and/or spanning the aforementioned values. In some embodiments, the soft chewable formulation includes about 30.0% w/w to about 55.0% w/w of a polymeric carbohydrate. In some embodiments, the soft chewable formulation includes about 35.0% w/w to about 50.0% w/w of a polymeric carbohydrate. In some embodiments, the soft chewable formulation includes about 40.0% w/w to about 50.0% w/w of a polymeric carbohydrate. In some embodiments, the soft chewable formulation includes about 30.0% w/w to about 40.0% w/w of a polymeric carbohydrate. In some embodiments, the soft chewable formulation includes about 35.0% w/w to about 45.0% w/w of a polymeric carbohydrate. In some embodiments, the soft chewable formulation includes about 34% w/w of a polymeric carbohydrate. In some embodiments, the soft chewable formulation includes about 36% w/w of a polymeric carbohydrate. In some embodiments, the soft chewable formulation includes about 37% w/w of a polymeric carbohydrate. In some embodiments, the soft chewable formulation includes about 38% w/w of a polymeric carbohydrate. In some embodiments, the soft chewable formulation includes about 39% w/w of a polymeric carbohydrate. In some embodiments, the soft chewable formulation includes about 40% w/w of a polymeric carbohydrate. In some embodiments, the soft chewable formulation includes about 42% w/w of a polymeric carbohydrate. In some embodiments, the soft chewable formulation includes about 43% w/w of a polymeric carbohydrate. In some embodiments, the soft chewable formulation includes about 44% w/w of a polymeric carbohydrate. In some embodiments, the soft chewable formulation includes about 45% w/w of a polymeric carbohydrate. In some embodiments, the soft chewable formulation includes about 46% w/w of a polymeric carbohydrate. In some embodiments, the soft chewable formulation includes about 47% w/w of a polymeric carbohydrate. In some embodiments, the soft chewable formulation includes about 48% w/w of a polymeric carbohydrate. In some embodiments, the soft chewable formulation includes about 51% w/w of a polymeric carbohydrate.

In some embodiments, the soft chewable formulation includes about 20% to about 50% w/w of a regular starch. In some embodiments, the soft chewable formulation includes about 20%, 22%, 24%, 26%, 28%, 30%, 32%, 34%, 36%, 38%, 40%, 42%, 44%, 46%, 48%, 50.0% w/w of a regular starch, or ranges including and/or spanning the aforementioned values. In some embodiments, the soft chewable formulation includes about 30.0% w/w to about 50.0% w/w of a regular starch. In some embodiments, the soft chewable formulation includes about 40.0% w/w to about 50.0% w/w of a regular starch. In some embodiments, the soft chewable formulation includes about 30.0% w/w to about 40.0% w/w of a regular starch. In some embodiments, the soft chewable formulation includes about 35.0% w/w to about 45.0% w/w of a regular starch. In some embodiments, the soft chewable formulation includes about 28% w/w of a regular starch. In some embodiments, the soft chewable formulation includes about 30% w/w of a regular starch. In some embodiments, the soft chewable formulation includes about 31% w/w of a regular starch. In some embodiments, the soft chewable formulation includes about 32% w/w of a regular starch. In some embodiments, the soft chewable formulation includes about 33% w/w of a regular starch. In some embodiments, the soft chewable formulation includes about 34% w/w of a regular starch. In some embodiments, the soft chewable formulation includes about 35% w/w of a regular starch. In some embodiments, the soft chewable formulation includes about 36% w/w of a regular starch. In some embodiments, the soft chewable formulation includes about 38% w/w of a regular starch. In some embodiments, the soft chewable formulation includes about 39% w/w of a regular starch. In some embodiments, the soft chewable formulation includes about 40% w/w of a regular starch. In some embodiments, the soft chewable formulation includes about 44% w/w of a regular starch. In some embodiments, the soft chewable formulation includes about 45% w/w of a regular starch. In some embodiments, the soft chewable formulation includes about 46% w/w of a regular starch. In some embodiments, the soft chewable formulation includes about 47% w/w of a regular starch.

In some embodiments, the soft chewable formulation includes about 2% to about 16% w/w of a pregelatinized starch. In some embodiments, the soft chewable formulation includes about 2%, 3%, 4%, 5%, 6%, 7%, 8%, 9%, 10%, 11%, 12%, 13%, 14%, 15%, 16% w/w of a pregelatinized starch, or ranges including and/or spanning the aforementioned values. In some embodiments, the soft chewable formulation includes about 2.0% w/w to about 8.0% w/w of a pregelatinized starch. In some embodiments, the soft chewable formulation includes about 3.0% w/w to about 8.0% w/w of a pregelatinized starch. In some embodiments, the soft chewable formulation includes about 2.0% w/w to about 6.0% w/w of pregelatinized starch. In some embodiments, the soft chewable formulation includes about 3.0% w/w to about 6.0% w/w of a pregelatinized starch. In some embodiments, the soft chewable formulation includes about 4.0% w/w to about 6.0% w/w of a pregelatinized starch. In some embodiments, the soft chewable formulation includes about 4% w/w of a pregelatinized starch. In some embodiments, the soft chewable formulation includes about 5% w/w of a pregelatinized starch. In some embodiments, the soft chewable formulation includes about 6% w/w of a pregelatinized starch. In some embodiments, the soft chewable formulation includes about 7% w/w of a pregelatinized starch. In some embodiments, the soft chewable formulation includes about 8% w/w of a pregelatinized starch.

In some embodiments, the soft chewable formulation includes a ratio of regular starch to pre-gelatinized starch from about 1:1, from about 2:1, from about 3:1, from about 4:1, from about 5:1, from about 6:1, from about 7:1, from about 8:1, from about 9:1, from about 10:1, from about 11:1, or ranges including and/or spanning the aforementioned values. In some embodiments, the soft chewable formulation includes a ratio of regular starch to pre-gelatinized starch from about 4:1. In some embodiments, the soft chewable formulation includes a ratio of regular starch to pre-gelatinized starch from about 5:1. In some embodiments, the soft chewable formulation includes a ratio of regular starch to pre-gelatinized starch from about 5.4:1. In some embodiments, the soft chewable formulation includes a ratio of regular starch to pre-gelatinized starch from about 5.5:1. In some embodiments, the soft chewable formulation includes a ratio of regular starch to pre-gelatinized starch from about 5.6:1. In some embodiments, the soft chewable formulation includes a ratio of regular starch to pre-gelatinized starch from about 6:1. In some embodiments, the soft chewable formulation includes a ratio of regular starch to pre-gelatinized starch from about 6.3:1. In some embodiments, the soft chewable formulation includes a ratio of regular starch to pre-gelatinized starch from about 6.4:1. In some embodiments, the soft chewable formulation includes a ratio of regular starch to pre-gelatinized starch from about 6.5:1. In some embodiments, the soft chewable formulation includes a ratio of regular starch to pre-gelatinized starch from about 6.6:1. In some embodiments, the soft chewable formulation includes a ratio of regular starch to pre-gelatinized starch from about 6.9:1. In some embodiments, the soft chewable formulation includes a ratio of regular starch to pre-gelatinized starch from about 7:1. In some embodiments, the soft chewable formulation includes a ratio of regular starch to pre-gelatinized starch from about 7.3:1. In some embodiments, the soft chewable formulation includes a ratio of regular starch to pre-gelatinized starch from about 7.4:1. In some embodiments, the soft chewable formulation includes a ratio of regular starch to pre-gelatinized starch from about 7.5:1. In some embodiments, the soft chewable formulation includes a ratio of regular starch to pre-gelatinized starch from about 7.75:1. In some embodiments, the soft chewable formulation includes a ratio of regular starch to pre-gelatinized starch from about 8:1. In some embodiments, the soft chewable formulation includes a ratio of regular starch to pre-gelatinized starch from about 8.25:1. In some embodiments, the soft chewable formulation includes a ratio of regular starch to pre-gelatinized starch from about 8.5:1. In some embodiments, the soft chewable formulation includes a ratio of regular starch to pre-gelatinized starch from about 8.75:1. In some embodiments, the soft chewable formulation includes a ratio of regular starch to pre-gelatinized starch from about 9:1. In some embodiments, the soft chewable formulation includes a ratio of regular starch to pre-gelatinized starch from about 9.2:1. In some embodiments, the soft chewable formulation includes a ratio of regular starch to pre-gelatinized starch from about 9.5:1. In some embodiments, the soft chewable formulation includes a ratio of regular starch to pre-gelatinized starch from about 9.75:1. In some embodiments, the soft chewable formulation includes a ratio of regular starch to pre-gelatinized starch from about 10:1. In some embodiments, the soft chewable formulation includes a ratio of regular starch to pre-gelatinized starch from about 10.3:1. In some embodiments, the soft chewable formulation includes a ratio of regular starch to pre-gelatinized starch from about 11:1. In some embodiments, the soft chewable formulation includes a ratio of regular starch to pre-gelatinized starch in a range from about 1:1 to about 11:1. In some embodiments, the soft chewable formulation includes a ratio of regular starch to pre-gelatinized starch in a range from about 5:1 to about 10:1. In some embodiments, the soft chewable formulation includes a ratio of regular starch to pre-gelatinized starch in a range from about 6:1 to about 11:1. In some embodiments, the soft chewable formulation includes a ratio of regular starch to pre-gelatinized starch in a range from about 7:1 to about 10:1. In some embodiments, the soft chewable formulation includes a ratio of regular starch to pre-gelatinized starch in a range from about 5:1 to about 9:1. In some embodiments, the soft chewable formulation includes a ratio of regular starch to pre-gelatinized starch in a range from about 6:1 to about 9:1.

In some embodiments, the soft chew formulation includes a combination of at least one active ingredient and a regular starch from about 20% to about 50% w/w. In some embodiments, the soft chew formulation includes a combination of at least one active ingredient and a regular starch from about 20%, 22%, 24%, 26%, 28%, 30%, 32%, 34%, 36%, 38%, 40%, 42%, 44%, 46%, 48%, 50.0% w/w, or ranges including and/or spanning the aforementioned values. In some embodiments, the soft chewable formulation includes about 25.0% w/w to about 50.0% w/w of a combination of at least one active ingredient and a regular starch. In some embodiments, the soft chewable formulation includes about 30.0% w/w to about 50.0% w/w of a combination of at least one active ingredient and a regular starch. In some embodiments, the soft chewable formulation includes about 30.0% w/w to about 40.0% w/w of a combination of at least one active ingredient and a regular starch. In some embodiments, the soft chewable formulation includes about 35.0% w/w to about 45.0% w/w of a combination of at least one active ingredient and a regular starch. In some embodiments, the soft chewable formulation includes about 40.0% w/w to about 50.0% w/w of a combination of at least one active ingredient and a regular starch. In some embodiments, the soft chewable formulation includes about 30.0% w/w to about 35.0% w/w of a combination of at least one active ingredient and a regular starch. In some embodiments, the soft chewable formulation includes about 35.0% w/w to about 4.0% w/w of a combination of at least one active ingredient and a regular starch. In some embodiments, the soft chew formulation includes a combination of at least one active ingredient and a regular starch from about 28%. In some embodiments, the soft chew formulation includes a combination of at least one active ingredient and a regular starch from about 25%. In some embodiments, the soft chew formulation includes a combination of at least one active ingredient and a regular starch from about 31%. In some embodiments, the soft chew formulation includes a combination of at least one active ingredient and a regular starch from about 32%. In some embodiments, the soft chew formulation includes a combination of at least one active ingredient and a regular starch from about 33%. In some embodiments, the soft chew formulation includes a combination of at least one active ingredient and a regular starch from about 34%. In some embodiments, the soft chew formulation includes a combination of at least one active ingredient and a regular starch from about 35%. In some embodiments, the soft chew formulation includes a combination of at least one active ingredient and a regular starch from about 37%. In some embodiments, the soft chew formulation includes a combination of at least one active ingredient and a regular starch from about 38%. In some embodiments, the soft chew formulation includes a combination of at least one active ingredient and a regular starch from about 39%. In some embodiments, the soft chew formulation includes a combination of at least one active ingredient and a regular starch from about 40%. In some embodiments, the soft chew formulation includes a combination of at least one active ingredient and a regular starch from about 41%. In some embodiments, the soft chew formulation includes a combination of at least one active ingredient and a regular starch from about 42%. In some embodiments, the soft chew formulation includes a combination of at least one active ingredient and a regular starch from about 49%.

In some embodiments, the soft chew formulation includes a ratio of at least one active ingredient and a regular starch to pregelatinized starch from about 4:1 to about 12:1. In some embodiments, the soft chewable formulation includes a ratio of at least one active ingredient and a regular starch to pregelatinized starch from about 6:1 to about 12:1. In some embodiments, the soft chewable formulation includes a ratio of at least one active ingredient and a regular starch to pregelatinized starch from about 8:1 to about 12:1. In some embodiments, the soft chewable formulation includes a ratio of at least one active ingredient and a regular starch to pregelatinized starch from about 10:1 to about 12:1. In some embodiments, the soft chewable formulation includes a ratio of at least one active ingredient and a regular starch to pregelatinized starch from about 5:1 to about 8:1. In some embodiments, the soft chewable formulation includes a ratio of at least one active ingredient and a regular starch to pregelatinized starch from about 6:1 to about 8:1. In some embodiments, the soft chew formulation includes a ratio of at least one active ingredient and a regular starch to pregelatinized starch from about 4:1 to about 9:1. In some embodiments, the soft chew formulation includes a ratio of at least one active ingredient and a regular starch to pregelatinized starch from about 4:1 to about 8:1. In some embodiments, the soft chew formulation includes a ratio of at least one active ingredient and a regular starch to pregelatinized starch from about 4:1 to about 7:1. In some embodiments, the soft chew formulation includes a ratio of at least one active ingredient and a regular starch to pregelatinized starch from about 4:1 to about 6:1. In some embodiments, the soft chew formulation includes a ratio of at least one active ingredient and a regular starch to pregelatinized starch from about 4:1 to about 5:1. In some embodiments, the soft chew formulation includes a ratio of at least one active ingredient and a regular starch to pregelatinized starch from about 5:1 to about 9:1. In some embodiments, the soft chew formulation includes a ratio of at least one active ingredient and a regular starch to pregelatinized starch from about 5:1 to about 8:1. In some embodiments, the soft chew formulation includes a ratio of at least one active ingredient and a regular starch to pregelatinized starch from about 5:1 to about 7:1. In some embodiments, the soft chew formulation includes a ratio of at least one active ingredient and a regular starch to pregelatinized starch from about 5:1 to about 6:1. In some embodiments, the soft chew formulation includes a ratio of at least one active ingredient and a regular starch to pregelatinized starch from about 4:1. In some embodiments, the soft chew formulation includes a ratio of at least one active ingredient and a regular starch to pregelatinized starch from about 5:1. In some embodiments, the soft chewable formulation includes a ratio of regular starch to pre-gelatinized starch from about 5.5:1. In some embodiments, the soft chew formulation includes a ratio of at least one active ingredient and a regular starch to pregelatinized starch from about 6:1. In some embodiments, the soft chew formulation includes a ratio of at least one active ingredient and a regular starch to pregelatinized starch from about 6.5:1. In some embodiments, the soft chew formulation includes a ratio of at least one active ingredient and a regular starch to pregelatinized starch from about 7:1. In some embodiments, the soft chew formulation includes a ratio of at least one active ingredient and a regular starch to pregelatinized starch from about 7.5:1. In some embodiments, the soft chew formulation includes a ratio of at least one active ingredient and a regular starch to pregelatinized starch from about 7.75:1. In some embodiments, the soft chew formulation includes a ratio of at least one active ingredient and a regular starch to pregelatinized starch from about 8:1. In some embodiments, the soft chew formulation includes a ratio of at least one active ingredient and a regular starch to pregelatinized starch from about 8.5:1. In some embodiments, the soft chew formulation includes a ratio of at least one active ingredient and a regular starch to pregelatinized starch from about 8.75:1. In some embodiments, the soft chew formulation includes a ratio of at least one active ingredient and a regular starch to pregelatinized starch from about 9:1. In some embodiments, the soft chew formulation includes a ratio of at least one active ingredient and a regular starch to pregelatinized starch from about 9.5:1. In some embodiments, the soft chew formulation includes a ratio of at least one active ingredient and a regular starch to pregelatinized starch from about 9.75:1. In some embodiments, the soft chew formulation includes a ratio of at least one active ingredient and a regular starch to pregelatinized starch from about 10:1. In some embodiments, the soft chew formulation includes a ratio of at least one active ingredient and a regular starch to pregelatinized starch from about 10.5:1.

In some embodiments, the soft chewable formulation includes a pharmaceutically acceptable excipient. In some embodiments, a pharmaceutically acceptable excipient may include a surfactant, a filler, a humectant, a solvent, a softening agent, a preservative, a lubricant, or a combination thereof. In some embodiments, the soft chewable formulation includes about 0.1% to about 15% w/w of a pharmaceutically acceptable excipient. In some embodiments, the soft chewable formulation includes about 0.1%, 1%, 2%, 3%, 4%, 4%, 5%, 6%, 7%, 8%, 9%, 10%, 11%, 12%, 13%, 14%, 15% w/w of a pharmaceutically acceptable excipient, or ranges including and/or spanning the aforementioned values. In some embodiments, the soft chewable formulation includes about 1.0% w/w to about 10.0% w/w of an excipient. In some embodiments, the soft chewable formulation includes about 5.0% w/w to about 15.0% w/w of an excipient. In some embodiments, the soft chewable formulation includes about 1.0% w/w to about 5.0% w/w of an excipient. In some embodiments, the soft chewable formulation includes about 2.0% w/w to about 6.0% w/w of an excipient. In some embodiments, the soft chewable formulation includes about 5.0% w/w to about 8.0% w/w of an excipient. In some embodiments, the soft chewable formulation includes about 6.0% w/w to about 15.0% w/w of an excipient.

In some embodiments, the soft chewable formulation includes a surfactant. Non-limiting examples of surfactants include compounds such as sodium caprylate, cetylpyridinium chloride, polysorbate 40, polysorbate 60, polysorbate 85, lauric acid, myristic acid, lauryl betaine, coco glucoside, glyceryl oleate, decyl glucoside, cocamidopropyl hydroxysultaine, sorbitan oleate, sorbitan palmitate, sorbitan trioleate, ceteareth-20, ceteareth-25, sodium lauroamphoacetate, sodium methyl cocoyl taurate, disodium lauroamphodiacetate, caprylic/capric triglyceride, lauramide DEA, behentrimonium methosulfate, oleth-10, oleth-20, sodium lauryl sulfate, sodium doccusate, triacetin, vitamin E TPGS, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropyl methylcellulose, hydroxypropylcellulose, polyvinylpyrrolidone, hydroxypropylmethylcellulose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 9085®, which is a tetrafunctional block copolymer derived from sequential addition of propylene oxide and ethylene oxide to ethylenediamine (BASF Wyandotte Corporation, Parsippany, N.J.)); Tetronic 1508® (T-1508, a poloxamine) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-IOG® or Surfactant 10-G® (Olin Chemicals, Stamford, Conn.); Crodestas SL-40® (Croda, Inc.); and SA90HCO, which is C18H37CH2C(O)N(CH3)-CH2(CHOH)4(CH2OH)2 (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, PEG-40 hydrogenated castor oil, PEG-20 sorbitan cocate, PEG-30 dipolyhydroxystearate, lysozyme, random copolymers of vinyl pyrrolidone and vinyl acetate. The above surfactants are commercially available or can be prepared by techniques known in the art. Many are described in detail in the Handbook of Pharmaceutical Excipients, published jointly by the American Pharmaceutical Association and The Pharmaceutical Society of Great Britain (The Pharmaceutical Press, 2009), specifically incorporated by reference. In some embodiments, an emulsifying agent is a surfactant.

In some embodiments, the soft chewable formulation includes an emulsifying agent. Non-limiting examples of emulsifying agents include, but are not limited to, calcium stearoyl lactylate (CSL), glycerol monosterate (GMS), potassium laurate, triethanolamine stearate, magnesium stearate, mono- and diglycerides, sodium lauryl sulfate, alkyl polyoxyethylene sulfates, sodium dodecyl sulfate, dioctyl sodium sulfosuccinate, sodium stearoyl lactylate (SSL), polyoxyethylene fatty acid derivatives of the sorbitan esters (for example, Tween series), polyoxyethylene fatty alcohol, ethers, sorbitan fatty acid esters, polyoxyethylene alkyl ethers (macrogels) polyoxyethylene sorbitan fatty acid esters, fatty alcohols, cetyl alcohols, stearyl alcohol, behenyl alcohol, phospholipids, ethoxylated mono- and diglycerides, lactylated fatty acid esters of glycerol and propylene glycol, polyglycerol esters of fatty acids, propylene glycol esters of fatty acids, stearoyl-2-lactylate, ammonium phosphatide, glyceryl oleate (monoolein), glyceryl polyricinoleate, polyoxyethylene polyoxypropylene block copolymers (poloxamers), polyethylene glycol 400 monostearate, sucrose esters, lanolin alcohols, hydrophilic colloids, alginates, acacia, tragacanth, xanthan, vitamin E, pectin, gelatin, casein, lecithin, or combinations thereof. In some embodiments, the emulsifying agent is lecithin powder. In some embodiments, the emulsifying agent is soy lecithin powder.

In some embodiments, the soft chewable formulation includes less than about 5.0% w/w of an emulsifying agent. In some embodiments, the soft chewable formulation includes less than about 4.0% w/w of an emulsifying agent. In some embodiments, the soft chewable formulation includes less than about 3.0% w/w of an emulsifying agent. In some embodiments, the soft chewable formulation includes about 0.1% to about 5% w/w of an emulsifying agent. In some embodiments, the soft chewable formulation includes about 0.1%, 0.5%, 1%, 1.5%, 2.0%, 2.5%, 3.0%, 3.5%, 4.0%, 4.5%, 5.0% w/w of an emulsifying agent, or ranges including and/or spanning the aforementioned values. In some embodiments, the soft chewable formulation includes about 1.0% w/w of an emulsifying agent. In some embodiments, the soft chewable formulation includes about 3.0% w/w of an emulsifying agent. In some embodiments, the soft chewable formulation includes about 4.0% w/w of an emulsifying agent. In some embodiments, the soft chewable formulation includes about 5.0% w/w of an emulsifying agent.

In some embodiments, the soft chewable formulation includes a disintegrant. For soft chewable formulations, all disintegrants known in the soft chewable formulation art are contemplated. Non-limiting examples of a disintegrant include a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, calcium carboxymethylcellulose, potassium polacrilin, low substituted hydroxypropylcellulose, polacrilin potassium, ammonium carbonate, ammonium bicarbonate, docustate sodium, talc, urea, magnesium silicate, fumaric acid, tartaric acid, citric acid, calcium sulfate, magnesium oxide, calcium phosphate, polyacrylates, polysorbates like polysorbate 80, croscarmellose, or a cross-linked cellulose, such as cross-linked sodium carboxymethylcellulose (Ac-Di-Sol®), cross-linked carboxymethylcellulose, or cross-linked croscarmellose, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate (Explotab®), bentonite, a natural sponge, a surfactant, a resin such as a cation-exchange resin, citrus pulp, sodium lauryl sulfate, and the like. In some embodiments, the disintegrant is croscarmellose. In some embodiments, the disintegrant is crospovidone.

In some embodiments, the soft chewable formulation includes about 0.1% to about 10% w/w of a disintegrant. In some embodiments, the soft chewable formulation includes about 0.1%, 0.5%, 1%, 1.5%, 2.0%, 2.5%, 3.0%, 3.5%, 4.0%, 4.5%, 5.0%, 6.0%, 7.0%, 8.0%, 9.0%, 10.0% w/w of a disintegrant, or ranges including and/or spanning the aforementioned values. In some embodiments, the soft chewable formulation includes about 0.50% w/w of a disintegrant. In some embodiments, the soft chewable formulation includes about 0.6% w/w of a disintegrant. In some embodiments, the soft chewable formulation includes about 1.0% w/w of a disintegrant. In some embodiments, the soft chewable formulation includes about 4.0% w/w of a disintegrant. In some embodiments, the soft chewable formulation includes about 5.0% w/w of a disintegrant. In some embodiments, the soft chewable formulation includes about 6.0% w/w of a disintegrant. In some embodiments, the soft chewable formulation includes about 7.0% w/w of a disintegrant. In some embodiments, the soft chewable formulation includes about 8.0% w/w of a disintegrant. In some embodiments, the soft chewable formulation includes about 9.0% w/w of a disintegrant. In some embodiments, the soft chewable formulation includes about 10.0% w/w of a disintegrant. In some embodiments, the soft chewable formulation includes about 1.0% w/w to about 10.0% w/w of a disintegrant. In some embodiments, the soft chewable formulation includes about 1.0% w/w to about 5.0% w/w of a disintegrant. In some embodiments, the soft chewable formulation includes about 0.5% w/w to about 5.0% w/w of a disintegrant. In some embodiments, the soft chewable formulation includes about 2.0% w/w to about 5.0% w/w of a disintegrant. In some embodiments, the soft chewable formulation includes about 3.0% w/w to about 6.0% w/w of a disintegrant.

In some embodiments, the one or more active ingredients is a nutrient. In some embodiments, the soft chewable formulation further includes a nutrient. In some embodiments, the nutrient is a nutritional supplement. In some embodiments, the nutrient includes a vitamin, amino acid, electrolyte, supplement, and mineral, or a combination thereof. Example nutrients include, but not limited to, thiamin, riboflavin, nicotinic acid, pyridoxine, pantothenic acid, biotin, flavin, choline, inositol and paraminobenzoic acid, carnitine, vitamin C, vitamin D and its analogs (such as ergocalciferol, calcitriol, doxercalciferol, and paricalcitol), vitamin A and the carotenoids, folate, niacin, B vitamins, iodine, omega-3 fatty acids, omega-6 fatty acids, amino acids, coenzyme Q10, carotenoids, retinoic acid, vitamin E and vitamin K, sodium, potassium, chloride, calcium, magnesium, phosphate, phosphorus, iron, zinc, chromium, enzymes, Echinacea, garlic, glucosamine, and probiotics.

In some embodiments, the soft chewable formulation includes about 0.1% to about 25% of a nutrient. In some embodiments, the soft chewable formulation includes about 0.1%, 0.5%, 1.0%, 2.5%, 5.0%, 7.5%, 10%, 12.5%, 15%, 17.5%, 20%, 22.5%, 25%, w/w of a nutrient, or ranges including and/or spanning the aforementioned values. In some embodiments, the soft chewable formulation includes about 1% w/w of a nutrient. In some embodiments, the soft chewable formulation includes about 4% w/w of a nutrient. In some embodiments, the soft chewable formulation includes about 8% w/w of a nutrient. In some embodiments, the soft chewable formulation includes about 12% w/w of a nutrient. In some embodiments, the soft chewable formulation includes about 16% w/w of a nutrient. In some embodiments, the soft chewable formulation includes about 19% w/w of a nutrient. In some embodiments, the soft chewable formulation includes about 24% w/w of a nutrient. In some embodiments, the soft chewable formulation includes about 1.0% w/w to about 5.0% w/w of a nutrient. In some embodiments, the soft chewable formulation includes about 0.1% w/w to about 2.5% w/w of a nutrient.

In some embodiments, the soft chewable formulation includes a lipid. In some embodiments, the lipid is a triglyceride, phospholipid, glycolipid, sterol, fatty acid, waxes and sterol esters, sphingolipids, surfactants, lipid nanoparticles, ethosomes, lipid complexes, lipid emulsions, or combinations thereof. In some embodiments, the lipid is a triglyceride. In some embodiments, triglyceride is a vegetable oil. In some embodiments, the lipid is a liquid vegetable oil. In some embodiments, the lipid is a solid hydrogenated vegetable oil. The vegetable oil may be, for example, soybean oil, olive oil, flaxseed oil, canola oil, sunflower oil, safflower oil, peanut oil, sesame oil, coconut oil, palm oil, grape seed oil, rice bran oil, walnut oil, almond oil, avocado oil, cottonseed oil, hazelnut oil, hemp seed oil, macadamia oil, pumpkin seed oil, argan oil, evening primrose oil, jojoba oil, or corn oil. In some embodiments, the vegetable oil is a refined soybean oil.

In some embodiments, the soft chewable formulation includes about 5% to about 15% w/w of a lipid. In some embodiments, the soft chewable formulation includes about 5%, 6%, 7%, 8%, 9%, 10%, 11%, 12%, 13%, 14%, 15% w/w of a lipid, or ranges including and/or spanning the aforementioned values. In some embodiments, the soft chewable formulation includes about 7% w/w of a lipid. In some embodiments, the soft chewable formulation includes about 8% w/w of a lipid. In some embodiments, the soft chewable formulation includes about 9% w/w of a lipid. In some embodiments, the soft chewable formulation includes about 10% w/w of a lipid. In some embodiments, the soft chewable formulation includes about 11% w/w of a lipid. In some embodiments, the soft chewable formulation includes about 5% w/w to about 15% w/w of a lipid. In some embodiments, the soft chewable formulation includes about 5% w/w to about 15% w/w of a lipid.

In some embodiments, the soft chewable formulation includes a flavoring agent. The flavoring agent may be animal-derived or synthetic. In various embodiments, the flavoring agent is an animal-derived agent, typically having a meat flavor. For example, suitable flavoring agents include, but not limited to, chicken, chicken liver powder, pork, pork liver powder, beef, beef liver powder, pork, poultry, fish (for example, salmon or tuna), or rawhide-derived products. In some embodiments, the flavoring agent may be a vegetable and plant-based flavors. In some embodiments, the vegetable and plant-based flavors may include, but not limited to, peanut butter, sweet potato, apple, carrot, pumpkin, blueberry, mint, or a combination thereof. In some embodiments, the flavoring agent may include a smoked and roasted flavor. In some embodiments, the smoked and roasted flavor may be selected from, but not limited to, hickory smoke, BBQ, or a combination thereof. In some embodiments, the flavoring agent may be selected from a natural extract and oils. In some embodiments, the natural extract and oils may be selected from, but not limited to, vanilla aniseed, fennel, parsley oil, or a combination thereof. In various embodiments, the soft chewable includes a pork liver powder flavoring agent. In other embodiments, the flavoring agent comprises a synthetic flavoring agent. In some embodiments, the flavoring agent is chicken liver powder. In some embodiments, the chicken liver powder is hydrolyzed. In some embodiments, the chicken liver powder is irradiated.

In some embodiments, the soft chewable formulation includes about 5.0% to about 15% w/w of a flavoring agent. In some embodiments, the soft chewable formulation includes about 5%, 6%, 7%, 8%, 9%, 10%, 11%, 12%, 13%, 14%, 15%, w/w of a flavoring agent, or ranges including and/or spanning the aforementioned values. In some embodiments, the soft chewable formulation includes about 10% w/w of a flavoring agent. In some embodiments, the soft chewable formulation includes about 12% w/w of a flavoring agent. In some embodiments, the soft chewable formulation includes about 7% w/w to about 12% w/w of a flavoring agent. In some embodiments, the soft chewable formulation includes about 10% w/w to about 15% w/w of a flavoring agent. In some embodiments, the soft chewable formulation includes about 10% w/w to about 12% w/w of a flavoring agent.

In some embodiments, the soft chewable formulation includes a humectant. In some embodiments, the humectant is selected from the group consisting of glycerin, glycerol triacetate, polydextrose, sorbitol, propylene glycol, ethylene glycol, butylene glycol, hyaluronic acid, maltitol, erythritol, isopropyl myristate, and lactic acid. In some embodiments, the humectant is glycerol. In some embodiments, the humectant is glycerin.

In some embodiments, the soft chewable formulation includes about 8% to about 20% w/w of a humectant. In some embodiments, the soft chewable formulation includes about 8%, 10%, 11%, 12%, 13%, 14%, 15%, 16%, 17%, 18%, 19%, 20%, w/w of a humectant, or ranges including and/or spanning the aforementioned values. In some embodiments, the soft chewable formulation includes about 11% w/w of a humectant. In some embodiments, the soft chewable formulation includes about 12% w/w of a humectant. In some embodiments, the soft chewable formulation includes about 13% w/w of a humectant. In some embodiments, the soft chewable formulation includes about 13.5% w/w of a humectant. In some embodiments, the soft chewable formulation includes about 14.5% w/w of a humectant. In some embodiments, the soft chewable formulation includes about 15% w/w of a humectant. In some embodiments, the soft chewable formulation includes about 15.5% w/w of a humectant. In some embodiments, the soft chewable formulation includes about 10% w/w to about 18% w/w of a humectant. In some embodiments, the soft chewable formulation includes about 11% w/w to about 16% w/w of a humectant. In some embodiments, the soft chewable formulation includes about 12% w/w to about 15% w/w of a humectant. In some embodiments, the soft chewable formulation includes about 13% w/w to about 15% w/w of a humectant.

In some embodiments, the soft chewable formulation includes a binder. In some embodiments, the binder is selected from the group consisting of polyvinylpyrrolidone, low molecular weight HPMC, and alginate.

In some embodiments, the soft chewable formulation includes about 0.1% to about 5% w/w of a binder. In some embodiments, the soft chewable formulation includes about 0.1%, 0.5%, 1%, 1.5%, 2.0%, 2.5%, 3.0%, 3.5%, 4.0%, 4.5%, 5.0% w/w of a binder, or ranges including and/or spanning the aforementioned values. In some embodiments, the soft chewable formulation includes about 0.50% w/w of a binder. In some embodiments, the soft chewable formulation includes about 1.0% w/w of a binder.

In some embodiments, the soft chewable formulation includes an antioxidant. In some embodiments, the antioxidant is selected from the group consisting of BHT (butylated hydroxy toluene), propyl gallate, ascorbic acid, ascorbyl palmitate, fumaric acid, malic acid, citric acid, edetic acid and its salts, lecithin, tartaric acid, sodium ascorbate, sodium metabisulfate, BHA (butylated hydroxy anisole), monothioglycerol, Tenox 2, Tenox PG, Tenox s-1, tocopherols (alpha-, beta-, or delta-tocopherol, tocopherol esters, alpha-tocopherol acetate), other alkyl gailates, resveratrol, quercetin, benzoic acid, Trolox (N-acetylcysteine, 6-hydroxy-2,5,7,8-tetramethylchroman-2-carboxylic acid), dimethyl thiourea (DMTU), hesperetin, tetrahydrocurcumin, tetrahydrodemethoxycurcumin, or combinations thereof.

In some embodiments, the soft chewable formulation includes about 0.1% to about 5% w/w of an antioxidant. In some embodiments, the soft chewable formulation includes about 0.1%, 0.5%, 1%, 1.5%, 2.0%, 2.5%, 3.0%, 3.5%, 4.0%, 4.5%, 5.0% w/w of an antioxidant, or ranges including and/or spanning the aforementioned values. In some embodiments, the soft chewable formulation includes about 2.5% w/w of an antioxidant. In some embodiments, the soft chewable formulation includes about 5.0% w/w of an antioxidant.

In some embodiments, the soft chewable formulation includes a preservative. In some embodiments, the soft chewable formulation includes two or more preservatives. For soft chewable formulations, all preservatives known in the soft chewable formulation art are contemplated. Non-limiting examples include the benzalkonium chloride, benzethonium chloride, benzoic acid, benzyl alcohol, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, propyl paraben, myristyl gamma-picolinium chloride, methylparaben, propylparaben and quaternary ammonium compounds and the like. In some embodiments, the preservative is one or more parabens. In some embodiments, the one or more parabens is selected from methylparaben and propylparaben. In some embodiments, the preservative is potassium sorbate powder.

In some embodiments, the soft chewable formulation includes about 0.05% to about 3.0% w/w of a preservative. In some embodiments, the soft chewable formulation includes about 0.05%, 0.06%, 0.07%, 0.08%, 0.09%, 0.1%, 0.11%, 0.12%, 0.13%, 0.14%, 0.15%, 0.25%, 0.50%, 0.6%, 0.7%, 0.8%, 0.9%, 1.0%, 1.25%, 1.5%, 1.75%, 2.0%, 2.25%, 2.5%, 2.75%, 3.0% w/w of a preservative, or ranges including and/or spanning the aforementioned values. In some embodiments, the soft chewable formulation includes about 0.1% w/w of a preservative. In some embodiments, the soft chewable formulation includes about 0.2% w/w of a preservative. In some embodiments, the soft chewable formulation includes about 0.5% w/w of a preservative. In some embodiments, the soft chewable formulation includes about 0.6% w/w of a preservative. In some embodiments, the soft chewable formulation includes about 0.75% w/w of a preservative. In some embodiments, the soft chewable formulation includes about 0.85% w/w of a preservative. In some embodiments, the soft chewable formulation includes about 0.95% w/w of a preservative. In some embodiments, the soft chewable formulation includes about 0.1% w/w to about 1.0% w/w of a preservative. In some embodiments, the soft chewable formulation includes about 0.1% w/w to about 1.0% w/w of a preservative. In some embodiments, the soft chewable formulation includes about 0.5% w/w to about 1.0% w/w of a preservative. In some embodiments, the soft chewable formulation includes about 0.1% w/w to about 0.5% w/w of a preservative.

In some embodiments, the soft chewable formulation includes pH stabilizers. Non-limiting examples include, acetic acid/acetate, malic acid/malate, citric acid/citrate, tataric acid/tartrate, lactic acid/lactate, phosphoric acid/phosphate, glycine/glycimate, tris, glutamic acid/glutamates and sodium carbonate. In some embodiments, the soft chewable formulation has a pH in a range from about 4.0 to about 7.0.

In some embodiments, the soft chewable formulation includes a coloring agent. Non-limiting examples include, organic dyes, lake pigments, natural colorants such as caramel, and mineral pigments based upon, for example, iron oxide or titanium dioxide. In some embodiments, the coloring agent is caramel color liquid.

In some embodiments, the soft chewable formulation includes about 0.1% to about 4.0% w/w of a coloring agent. In some embodiments, the soft chewable formulation includes about 0.1%, 0.2%, 0.4%, 0.6%, 0.8%, 1.0%, 1.2%, 1.4%, 1.6%, 1.8%, 2.0%, 2.5%, 3.0%, 3.5%, 4.0% w/w of a coloring agent, or ranges including and/or spanning the aforementioned values. In some embodiments, the soft chewable formulation includes about 0.3% w/w of a coloring agent. In some embodiments, the soft chewable formulation includes about 0.5% w/w of a coloring agent. In some embodiments, the soft chewable formulation includes about 1.0% w/w of a coloring agent. In some embodiments, the soft chewable formulation includes about 1.5% w/w of a coloring agent. In some embodiments, the soft chewable formulation includes about 2.0% w/w of a coloring agent. In some embodiments, the soft chewable formulation includes about 3.0% w/w of a coloring agent. In some embodiments, the soft chewable formulation includes about 4.0% w/w of a coloring agent. In some embodiments, the soft chewable formulation includes about 1.0% w/w to about 4.0% w/w of a coloring agent. In some embodiments, the soft chewable formulation includes about 1.0% w/w to about 3.0% w/w of a coloring agent. In some embodiments, the soft chewable formulation includes about 1.0% w/w to about 2.0% w/w of a coloring agent. In some embodiments, the soft chewable formulation includes about 0.5% w/w to about 2.5% w/w of a coloring agent.

In some embodiments, the soft chewable formulation includes a plasticizer. In some embodiments, a plasticizer may be selected from alcohols, glycols (such as propylene glycol), lanolin, wool fat, liquid paraffin, mineral oil, petrolatum, benzyl phenylformate, chlorobutanol, diethyl phthalate, glycerol, polyethylene glycol, propylene glycol, sorbitol, triacetin, benzyl phenyl formate, dibutyl sebacate, tributyl citrate, triethyl citrate, or any combination of any two or more thereof. In some embodiments, the plasticizer is sorbitol.

In some embodiments, the soft chewable formulation includes about 1.0% to about 10% w/w of a plasticizer. In some embodiments, the soft chewable formulation includes about 1%, 2%, 3%, 4%, 5%, 6%, 7%, 8%, 9%, 10% w/w of a plasticizer, or ranges including and/or spanning the aforementioned values. In some embodiments, the soft chewable formulation includes about 3% w/w of a plasticizer. In some embodiments, the soft chewable formulation includes about 4% w/w of a plasticizer. In some embodiments, the soft chewable formulation includes about 5% w/w of a plasticizer. In some embodiments, the soft chewable formulation includes about 6% w/w of a plasticizer. In some embodiments, the soft chewable formulation includes about 7% w/w of a plasticizer. In some embodiments, the soft chewable formulation includes about 2% w/w to about 10% w/w of a plasticizer. In some embodiments, the soft chewable formulation includes about 3% w/w to about 7% w/w of a plasticizer. In some embodiments, the soft chewable formulation includes about 4% w/w to about 8% w/w of a plasticizer. In some embodiments, the soft chewable formulation includes about 3% w/w to about 6% w/w of a plasticizer. In some embodiments, the soft chewable formulation includes about 1% w/w to about 5% w/w of a plasticizer.

In some embodiments, the soft chewable formulation includes a sweetener. In some embodiments, the sweetener may include a natural sweetener, a synthetic sweetener, or a combination thereof. Non-limiting examples of sweeteners include honey, maple syrup, corn syrup, agave nectar, molasses, date sugar, stevia, monk fruit, aspartame, sucralose, saccharain, acesulfame potassium, neotame, high-fructose corn syrup, sorbitol, xylitol, erythritol, mannitol, coconut sugar, barley malt syrup, and combinations thereof. In some embodiments, the sweetener is a corn syrup.

In some embodiments, the soft chewable formulation includes about 1.0% to about 10% w/w of a sweetener. In some embodiments, the soft chewable formulation includes about 1%, 2%, 3%, 4%, 5%, 6%, 7%, 8%, 9%, 10% w/w of a sweetener, or ranges including and/or spanning the aforementioned values. In some embodiments, the soft chewable formulation includes about 3% w/w of a sweetener. In some embodiments, the soft chewable formulation includes about 4% w/w of a sweetener. In some embodiments, the soft chewable formulation includes about 5% w/w of a sweetener. In some embodiments, the soft chewable formulation includes about 6% w/w of a sweetener. In some embodiments, the soft chewable formulation includes about 7% w/w of a sweetener. In some embodiments, the soft chewable formulation includes about 3% w/w to about 7% w/w of a sweetener. In some embodiments, the soft chewable formulation includes about 4% w/w to about 8% w/w of a sweetener. In some embodiments, the soft chewable formulation includes about 1% w/w to about 5% w/w of a sweetener. In some embodiments, the soft chewable formulation includes about 4% w/w to about 10% w/w of a sweetener.

In some embodiments, the soft chewable formulation includes a softening agent. Softening agents include those which limit density and hardness of the soft chew formulation. Such agents may include as non-limiting examples polysaccharides and fiber. A polysaccharide may be included in the form of a complex food such as a fruit, a plant starch such as potato or tapioca starch. Polysaccharide may also be provided separately, for example, in the form of chondroitin sulfate or glucosamine HCl.

In certain embodiments, the soft chewable formulation includes a wetting agent. A wetting agent is a chemical additive which reduces the surface tension of a fluid, inducing it to spread readily on a surface to which it is applied, thus causing even “wetting” of the surface with the fluids. Wetting agents provide a means for the liquid formulation to achieve intimate contact with the mucous membrane or other surface areas with which the pharmaceutical formulation comes in contact. In some embodiments, the wetting agent may be sodium lauryl sulfate, a pharmaceutically acceptable salt of docusate, and mixtures thereof.

In some embodiments, the soft chewable formulation of any of the preceding embodiments may include one or more attributes selected from: (i) the soft chew formulation is stable to long-term storage at various temperatures as described herein; (ii) the soft chew formulation is stable to humidity as described herein; (iii) the soft chew formulation is pH stable as described herein; (iii) the soft chew formulation is stable with one or more active ingredients; (iv) the soft chew formulation is stable even with up to +10% variation in the formulated API concentrations, as described herein; (v) the soft chew formulation is capable of retaining a similar degree of dissolution profile and softness that was found at the time of manufacturing; and (vi) the soft chew formulation is stable to long-term storage with a similar degree of palatability that was initially found at the time of manufacturing.

In some embodiments, the soft chewable formulation has an added water content of less than or equal to 5%. In some embodiments, the soft chewable formulation has an added water content of less than or equal to 4%. In some embodiments, the soft chewable formulation has an added water content of less than or equal to 3%. In some embodiments, the soft chewable formulation has an added water content of less than or equal to 2%. In some embodiments, the soft chewable formulation has an added water content of less than or equal to 1%. In some embodiments, the soft chewable formulation does not include added water. In some embodiments, the soft chewable formulation has a moisture content less than 10%. In some embodiments, the soft chewable formulation has a moisture content less than 8%. In some embodiments, the soft chewable formulation has a moisture content less than 6%. In some embodiments, the soft chewable formulation has a moisture content less than 4%. In some embodiments, the soft chewable formulation has a moisture content less than 2%. In some embodiments, the soft chewable formulation has a moisture content from about 1% w/w to about 10% w/w. In some embodiments, the soft chewable formulation has a moisture content from about 1% w/w to about 5% w/w. In some embodiments, the soft chewable formulation has a moisture content from about 2% w/w to about 8% w/w. In some embodiments, the soft chewable formulation has a moisture content from about 5% w/w to about 1% w/w.

In some embodiments, the active ingredient in the soft chew formulation is at least about or equal to 90% stable under 25° C. and 60% relative humidity conditions after 1 month. In some embodiments, the active ingredient in the soft chew formulation is at least about or equal to 90% stable under 25° C. and 60% relative humidity conditions after 2 months. In some embodiments, the active ingredient in the soft chew formulation is at least about or equal to 90% stable under 25° C. and 60% relative humidity conditions after 3 months. In some embodiments, the active ingredient in the soft chew formulation is at least about or equal to 90% stable under 25° C. and 60% relative humidity conditions after 4 months. In some embodiments, the active ingredient in the soft chew formulation is at least about or equal to 90% stable under 25° C. and 60% relative humidity conditions after 5 months. In some embodiments, the active ingredient in the soft chew formulation is at least about or equal to 90% stable under 25° C. and 60% relative humidity conditions after 6 months. In some embodiments, the active ingredient in the soft chew formulation is at least about or equal to 90% stable under 25° C. and 60% relative humidity conditions after 36 months.

In some embodiments, the active ingredient in the soft chew formulation is at least about or equal to 95% stable under 25° C. and 60% relative humidity conditions after 1 month. In some embodiments, the active ingredient in the soft chew formulation is at least about or equal to 95% stable under 25° C. and 60% relative humidity conditions after 2 months. In some embodiments, the active ingredient in the soft chew formulation is at least about or equal to 95% stable under 25° C. and 60% relative humidity conditions after 3 months. In some embodiments, the active ingredient in the soft chew formulation is at least about or equal to 95% stable under 25° C. and 60% relative humidity conditions after 4 months. In some embodiments, the active ingredient in the soft chew formulation is at least about or equal to 95% stable under 25° C. and 60% relative humidity conditions after 5 months. In some embodiments, the active ingredient in the soft chew formulation is at least about or equal to 95% stable under 25° C. and 60% relative humidity conditions after 6 months. In some embodiments, the active ingredient in the soft chew formulation is at least about or equal to 95% stable under 25° C. and 60% relative humidity conditions after 36 months.

In some embodiments, the active ingredient in the soft chew formulation is at least about or equal to 98% stable under 25° C. and 60% relative humidity conditions after 1 month. In some embodiments, the active ingredient in the soft chew formulation is at least about or equal to 98% stable under 25° C. and 60% relative humidity conditions after 2 months. In some embodiments, the active ingredient in the soft chew formulation is at least about or equal to 98% stable under 25° C. and 60% relative humidity conditions after 3 months. In some embodiments, the active ingredient in the soft chew formulation is at least about or equal to 98% stable under 25° C. and 60% relative humidity conditions after 4 months. In some embodiments, the active ingredient in the soft chew formulation is at least about or equal to 98% stable under 25° C. and 60% relative humidity conditions after 5 months. In some embodiments, the active ingredient in the soft chew formulation is at least about or equal to 98% stable under 25° C. and 60% relative humidity conditions after 6 months. In some embodiments, the active ingredient in the soft chew formulation is at least about or equal to 98% stable under 25° C. and 60% relative humidity conditions after 36 months.

In some embodiments, the active ingredient in the soft chew formulation is at least about or equal to 90% stable under 40° C. and 75% relative humidity conditions after 1 month. In some embodiments, the active ingredient in the soft chew formulation is at least about or equal to 90% stable under 40° C. and 75% relative humidity conditions after 2 months. In some embodiments, the active ingredient in the soft chew formulation is at least about or equal to 90% stable under 40° C. and 75% relative humidity conditions after 3 months. In some embodiments, the active ingredient in the soft chew formulation is at least about or equal to 90% stable under 40° C. and 75% relative humidity conditions after 4 months. In some embodiments, the active ingredient in the soft chew formulation is at least about or equal to 90% stable under 40° C. and 75% relative humidity conditions after 5 months. In some embodiments, the active ingredient in the soft chew formulation is at least about or equal to 90% stable under 40° C. and 75% relative humidity conditions after 6 months.

In some embodiments, the active ingredient in the soft chew formulation is at least about or equal to 95% stable under 40° C. and 75% relative humidity conditions after 1 month. In some embodiments, the active ingredient in the soft chew formulation is at least about or equal to 95% stable under 40° C. and 75% relative humidity conditions after 2 months. In some embodiments, the active ingredient in the soft chew formulation is at least about or equal to 95% stable under 40° C. and 75% relative humidity conditions after 3 months. In some embodiments, the active ingredient in the soft chew formulation is at least about or equal to 95% stable under 40° C. and 75% relative humidity conditions after 4 months. In some embodiments, the active ingredient in the soft chew formulation is at least about or equal to 95% stable under 40° C. and 75% relative humidity conditions after 5 months. In some embodiments, the active ingredient in the soft chew formulation is at least about or equal to 95% stable under 40° C. and 75% relative humidity conditions after 6 months.

In some embodiments, the active ingredient in the soft chew formulation is at least about or equal to 98% stable under 40° C. and 75% relative humidity conditions after 1 month. In some embodiments, the active ingredient in the soft chew formulation is at least about or equal to 98% stable under 40° C. and 75% relative humidity conditions after 2 months. In some embodiments, the active ingredient in the soft chew formulation is at least about or equal to 98% stable under 40° C. and 75% relative humidity conditions after 3 months. In some embodiments, the active ingredient in the soft chew formulation is at least about or equal to 98% stable under 40° C. and 75% relative humidity conditions after 4 months. In some embodiments, the active ingredient in the soft chew formulation is at least about or equal to 98% stable under 40° C. and 75% relative humidity conditions after 5 months. In some embodiments, the active ingredient in the soft chew formulation is at least about or equal to 98% stable under 40° C. and 75% relative humidity conditions after 6 months.

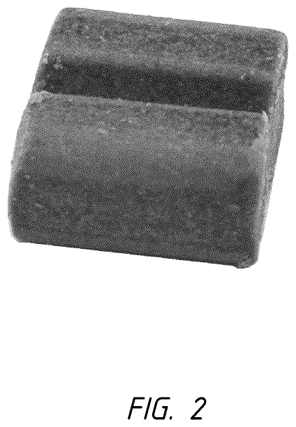

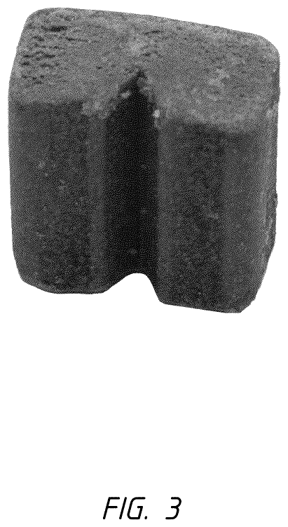

In some embodiments, the soft chew formulation can be formed as an edible pellet having any suitable shape (e.g., generally round, generally square, generally cuboid, or generally rectangular, or any combination, or other shape) or any suitable size, such that it is configured to fit within the mouth of the intended consuming animal for quick and easy mastication within the mouth without causing damage or discomfort. For example, the edible pellet can be less than or equal to about an inch in diameter, width, and/or length. FIGS. 2 and 3 illustrate an embodiment of a soft chew formulation having a generally square shape.

Before insertion into the mouth or mastication, the edible pellet can comprise an exterior surface that is sufficiently hard so as to retain the shape of the edible pellet as manufactured and so as to keep the material of the edible pellet together during packaging, shipping, storage, and holding or handling. In some embodiments, before insertion into the mouth, the edible pellet does not crumble, fracture, flake off, and/or morph its shape under normal forces or pressures incident to these activities, such as under any normal forces or pressures that are less than a typical range of bite, chew, or mastication forces or pressures for the intended consuming animal. For example, a normal mastication pressure by a dog while eating dog food can be at least about 50 pounds per square inch.

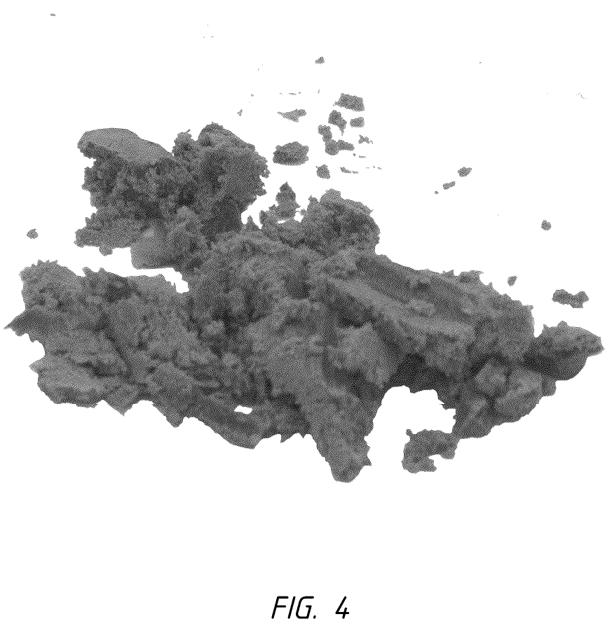

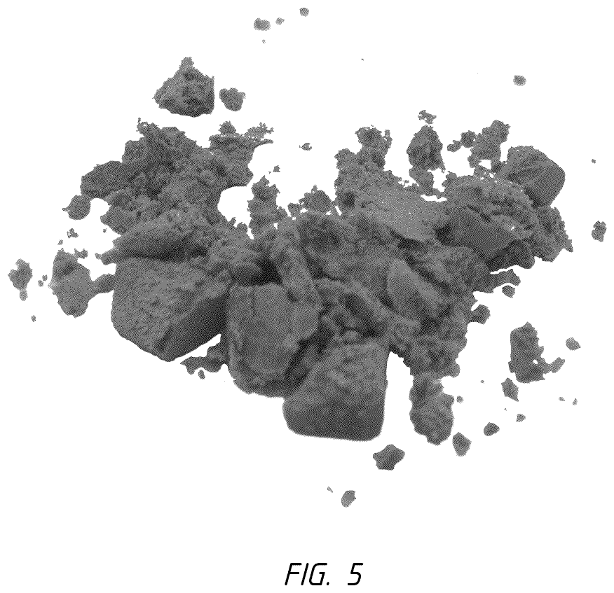

Upon or after an initial bite or chew by the intended consuming animal, which applies a range of typical mastication forces or pressures for such consuming animal, the hard exterior surface of the edible pellet can rapidly disintegrate, relent, or dissipate such that the edible pellet transforms into a soft, grainy amalgam. FIGS. 4 and 5 illustrate an embodiment of a soft chew formulation having been crushed into a soft and grainy amalgam. The majority of the grains of such amalgam can be small, such as less than or equal to about 0.5 mm or less than or equal to about 0.1 mm. The grains of the amalgam can tend to remain loosely together such that under low-level compression (e.g., less than or equal to about 10 pounds per square inch), they stick to each other without reforming a hard exterior surface. In some embodiments, after the initial bite or chew by the consuming animal, the former edible pellet does not produce any amount of hard or sharp portions or fragments that present any discomfort, or clinically significant risk of harm to the mouth or digestive system of the consuming animal, or that require long additional chewing (e.g., at least about 3 seconds) to eliminate.

Some aspects provide for an edible pellet with one or more active agents. In some embodiments, the edible pellet may be configured to be consumed by a predetermined type of animal. In some embodiments, the edible pellet comprises a matrix comprising an outer surface with a predefined shape that is sufficiently hard or durable so as to retain the predefined shape during packaging, shipping, storage, and pre-consumption handling without crumbling, flaking, or fracturing, the matrix comprising one or more active agents. In some embodiments, the matrix is chewable such that upon initial mastication in the mouth of the animal, applying a typical mastication pressure by animals of this type, the hard outer surface of the matrix disintegrates and the matrix transforms into a soft grainy amalgam. In some embodiments, the edible matrix retains its hardness and chewability properties for a shelf-life of at least two years. In some embodiments, the one or more active agents is uniformly dispersed within the matrix. In some embodiments, the matrix further comprises a regular starch and a pre-gelatinized starch in a ratio from about 3:1 to about 15:1. In some embodiments, the matrix further comprises at least one of one or more carbohydrates, a flavoring agent, a disintegrant, a lipid, a flavoring agent, a humectant, a preservative, a binder, an emulsifying agent, a sweetener, a coloring agent, surfactants, wetting agents, pH stabilizers, a softening agent, and a solvent. In some embodiments, the soft grainy amalgam induces the animal to chew thereupon.

In some embodiments, the edible pellet will maintain is predefined shape after 200 newtons of force is applied to the edible pellet. In some embodiments, the edible pellet will maintain is predefined shape after 250 newtons of force is applied to the edible pellet. In some embodiments, the edible pellet will maintain is predefined shape after 300 newtons of force is applied to the edible pellet. In some embodiments, the edible pellet will maintain is predefined shape after 350 newtons of force is applied to the edible pellet. In some embodiments, the edible pellet will maintain is predefined shape after 400 newtons of force is applied to the edible pellet. In some embodiments, the edible pellet will maintain is predefined shape after 450 newtons of force is applied to the edible pellet. In some embodiments, the edible pellet will maintain is predefined shape after 500 newtons of force is applied to the edible pellet. In some embodiments, the edible pellet will maintain is predefined shape after 550 newtons of force is applied to the edible pellet. In some embodiments, the edible pellet will maintain is predefined shape after 600 newtons of force is applied to the edible pellet.

In some embodiments, the matrix exhibits sufficient friability upon mastication in the mouth of the animal that the edible pellet is consumed by the animal in not more than about 2 seconds of chewing time by the majority of such animals. In some embodiments, the matrix exhibits sufficient friability upon mastication in the mouth of the animal that the edible pellet is consumed by the animal in not more than about 3 seconds of chewing time. In some embodiments, the matrix exhibits sufficient friability upon mastication in the mouth of the animal that the edible pellet is consumed by the animal in not more than about 4 seconds of chewing time. In some embodiments, the matrix exhibits sufficient friability upon mastication in the mouth of the animal that the edible pellet is consumed by the animal in not more than about 5 seconds of chewing time. In some embodiments, the matrix exhibits sufficient friability upon mastication in the mouth of the animal that the edible pellet is consumed by the animal in not more than about 6 seconds of chewing time. In some embodiments, the matrix exhibits sufficient friability upon mastication in the mouth of the animal that the edible pellet is consumed by the animal in not more than about 7 seconds of chewing time. In some embodiments, the matrix exhibits sufficient friability upon mastication in the mouth of the animal that the edible pellet is consumed by the animal in not more than about 8 seconds of chewing time. In some embodiments, the matrix exhibits sufficient friability upon mastication in the mouth of the animal that the edible pellet is consumed by the animal in not more than about 9 seconds of chewing time. In some embodiments, the matrix exhibits sufficient friability upon mastication in the mouth of the animal that the edible pellet is consumed by the animal in not more than about 10 seconds of chewing time.

In some embodiments, the matrix has a particle or granular material size from about 50 microns to about 1000 microns. In some embodiments, the matrix has a particle or granular material size from about 100 microns to about 1000 microns. In some embodiments, the matrix has a particle or granular material size from about 250 microns to about 1000 microns. In some embodiments, the matrix has a particle or granular material size from about 500 microns to about 1000 microns. In some embodiments, the amalgam has a particle or granular material size from 50 microns to 1000 microns. In some embodiments, the amalgam has a particle or granular material size from 100 microns to 1000 microns. In some embodiments, the amalgam has a particle or granular material size from 250 microns to 1000 microns. In some embodiments, the amalgam has a particle or granular material size from 500 microns to 1000 microns.

In some embodiments, the edible pellet retains its moldable properties for at least about one month. In some embodiments, the edible pellet retains its moldable properties for at least about two months. In some embodiments, the edible pellet retains its moldable properties for at least about three months. In some embodiments, the edible pellet retains its moldable properties for at least about six months. In some embodiments, the edible pellet retains its moldable properties for at least about one year. In some embodiments, the edible pellet retains its moldable properties for at least about two years.

### Preparation Process

Aspects of the disclosure also provide for a process for preparing a soft chewable formulation as described herein. In some embodiments, the process includes the steps of screening the starch, sugar, and the emulsifying agent through a mesh screen to form a dry raw material, mixing the dry raw material, adding the oil to the dry raw material to form a wetted raw material, mixing the wetted raw material, adding the at least one active anti-parasitic active ingredient to the wetted raw material to form a dough, and extruding the dough to form a chewable veterinary formulation.

In some embodiments, the process comprises screening all dry ingredients and mixing all the dry ingredients. In some embodiments, the liquid preservatives are subsequently added to the dry ingredients and mixed together. Next, glycerin, a coloring agent, and a sweetener are mixed in while a disintegrant is slowly added to the mixture. Next, the mixture is mixed to allow thorough mixing to occur of all the materials as the soft dough matrix of the disclosure is formed. Next, the dough is extruded and cut to individual doses of active drug and packaged.

### Method/Uses

Aspects disclosed herein relate to administering to a subject in need an effective amount of a soft chewable formulation as disclosed elsewhere herein. In some embodiments, the soft chewable formulation as described herein may be provided or administered to a subject to treat, prevent, control, or ameliorate a disease or condition in a subject in need thereof.

In some embodiments, the soft chewable formulation as described herein provides a very high level of bioavailability of the active ingredient after oral administration to the animal. Thus, depending on the active ingredient included in the formulations as described herein, the disclosure provides methods and uses for the treatment and prevention of various infections and diseases found in mammals, including animals. In some embodiments, the soft chewable formulation may be administered to a subject to treat, prevent, control or ameliorate an infection in a subject in need thereof.

In some embodiments, the soft chewable formulation may be provided at an effective amount to treat, prevent, control, delay the onset of, reduce the symptoms of, or ameliorate pain in a subject. In some embodiments, the soft chewable formulation may be provided at an effective amount to reduce the symptoms of pain in a subject. In some embodiments, the soft chewable formulation may be provided at an effective amount to retard the progression of pain in a subject. In some embodiments, the pain is chronic pain. In some embodiments, the pain is joint pain. In some embodiments, the pain is associated with inflammation. In some embodiments, the pain is associated with osteoarthritis. In some embodiments, the pain is associated with postoperative pain. In some embodiments, the pain is associated with soft-tissue in a subject.

In some embodiments, the soft chewable formulation may be provided at an effective amount to treat, prevent, control, delay the onset of, reduce the symptoms of, or ameliorate heart disease in a subject. In some embodiments, the soft chewable formulation may be provided at an effective amount to reduce the symptoms of heart disease in a subject. In some embodiments, the soft chewable formulation may be provided at an effective amount to retard the progression of heart disease in a subject. In some embodiments, the heart disease may be chronic heart disease. In some embodiments, the soft chewable formulation may be provided at an effective amount to treat, prevent, control, delay the onset of, reduce the symptoms of, or ameliorate coronary artery disease in a subject.

In some embodiments, the soft chewable formulation may be provided at an effective amount to treat, prevent, control, delay the onset of, reduce the symptoms of, or ameliorate nausea and vomiting in a subject. In some embodiments, the soft chewable formulation may be provided at an effective amount to reduce the symptoms of nausea and vomiting in a subject. In some embodiments, the soft chewable formulation may be provided at an effective amount to retard the progression of nausea and vomiting in a subject. In some embodiments, the soft chewable formulation may be provided at an effective amount to prevent, reduce or treat motion sickness. In some embodiments, the soft chewable formulation may be provided at an effective amount to prevent, reduce or treat gastroenteritis. In some embodiments, the soft chewable formulation may be provided at an effective amount to prevent, reduce or treat side effects of other drugs administered to the subject.

In some embodiments, the soft chewable formulation may be provided at an effective amount to treat, prevent, control, or ameliorate a viral infection or condition. In some embodiments, the viral infection or condition is selected from Ebola and Marburg virus (Filoviridae); Ross River virus, chikungunya virus, Sindbis virus, eastern equine encephalitis virus (Togaviridae, Alphavirus), vesicular stomatitis virus (Rhabdoviridae, Vesiculovirus), Amapari virus, Pichinde virus, Tacaribe virus, Junin virus, Machupo virus (Arenaviridae, Mammarenavirus), West Nile virus, dengue virus, yellow fever virus (Flaviviridae, Flavivirus); Moloney murine leukemia virus (Retroviridae, Gammaretrovirus); influenza A virus (Orthomyxoviridae); respiratory syncytial virus (Paramyxoviridae, Pneumovirinae, Pneumovirus); vaccinia virus (Poxviridae, Chordopoxvirinae, Orthopoxvirus); herpes simplex virus type 1, herpes simplex virus type 2 (Herpesviridae, Alphaherpesvirinae, Simplexvirus); Autographa californica nucleopolyhedrovirus (Baculoviridae, Alphabaculoviridae) (an insect virus); Ebola and Marburg virus (Filoviridae); Semliki Forest virus, Ross River virus, chikungunya virus, O'nyong-nyong virus, Sindbis virus, eastern/western/Venezuelan equine encephalitis virus (Togaviridae, Alphavirus); rubella (German measles) virus (Togaviridae, Rubivirus); rabies virus, Lagos bat virus, Mokola virus (Rhabdoviridae, Lyssavirus); Amapari virus, Pichinde virus, Tacaribe virus, Junin virus, Machupo virus, Guanarito virus, Sabia virus, Lassa virus (Arenaviridae, Mammarenavirus); West Nile virus, dengue virus, yellow fever virus, Zika virus, Japanese encephalitis virus, St. Louis encephalitis virus, tick-borne encephalitis virus, Omsk hemorrhagic fever virus, Kyasanur Forest virus (Flaviviridae, Flavivirus); influenza AB virus (Orthomyxoviridae, the common ‘flu’ virus); respiratory syncytial virus (Paramyxoviridae, Pneumovirinae, Pneumovirus); Hendra virus, Nipah virus (Paramyxoviridae, Paramyxovirinae, Henipavirus); measles virus (Paramyxoviridae, Paramyxovirinae, Morbillivirus); variola major (smallpox) virus (Poxviridae, Chordopoxvirinae, Orthopoxvirus); hepatitis delta virus (hepatitis D virus) (unassigned Family, Deltavirus); herpes simplex virus type 1, herpes simplex virus type 2 (Herpesviridae, Alphaherpesvirinae, Simplexvirus); canine distemper, canine influenza, canine parvovirus, herpes, feline calicivirus, coronavirus, feline leukemia virus, feline panleukopenia virus, feline valicivirus, feline herpes virus, varicella zoster virus, cytomegalovirus, or Epstein-Barr virus.

In some embodiments, the soft chewable formulation may be provided at an effective amount to treat, prevent, control, or ameliorate a bacterial infection or condition. In some embodiments, the soft chewable formulation may be provided at an effective amount to treat, prevent, control, or ameliorate a pathogenic or non-pathogenic microbes. Non-pathogenic microbes can, for example, cause colonization of a host without causing or producing any disease or disorder of the host. The microbial infection or colonization may be prokaryotic or eukaryotic, or a combination of both. Examples of prokaryotic microbes include bacteria and archaea. Examples of eukaryotic microbes include protists (such as algae, and slime-molds), fungi, multicellular micro-animals and plants including green algaes. Non-limiting examples of bacteria include gram positive bacteria, gram negative bacteria, biofilm-forming bacteria, extracellular bacteria, intracellular bacteria (including facultative and obligate intracellular bacteria), aerobic bacteria, and anaerobic bacteria. Some bacterial genera of interest, without limitation, include Bacillus, Bartonella, Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia. Some bacterial species of interest, without limitation, include Bacillus anthracia, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumonia, Chlamydia trachomatis, Chlamydophila psittaci, Clostridium botulinum, Clostridium difficile, Clostridium perfringens, Clostridium tetani, Corynebacterium diphtheria, Enterococcus faecalis, Enterococcus faecium, Escherichia coli, Francisella tularensis, Haemophilus influenza, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumonia, Neisseria gonorrhoeae, Neisseria meningitides, Pseudomonas aeruginosa, Rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumonia, Streptococcus pyogenes, Treponema pallidum, Ureaplasma urealyticum, Vibrio cholera, Yersinia pestis, Yersinia enterocolitica, Yersinia pseudotuberculosis.

In some embodiments, the soft chewable formulation may be provided at an effective amount to treat, prevent, control, or ameliorate a fungal disease or condition. In some embodiments, the soft chewable formulation may be provided at an effective amount to treat, prevent, control, or ameliorate a fungal organism or condition caused by a fungal organism. Examples of fungal disease or condition include, but are not limited to, Histoplasma capsulatum (causes Histoplasmosis), Blastomyces dermatitidis (causes Blastomycosis), and Coccidioides immitis (causes Coccidioidomycosis (Valley Fever)), Aspergillosis fumigatus (causes Aspergillosis), Aspergillus terreus (causes Disseminated Aspergillosis), Aspergillus deflectus, Aspergillus niger, Candida albicans (causes Candidiasis), Cryptococcus neoformans (causes Cryptococcosis), Cryptococcus gatti Geotrichum candidum (causes Geotrichosis), Pythium insidiosum (causes Oomycosis also called pythiosis), Rhinosporidium seeberi (causes Rhinosporidiosis), and Sporothrix schenckii (causes Sporotrichosis).

In some embodiments, the soft chewable formulation may be used for treating a parasitic, insect, acarid, or helminth infestation. In some embodiments, the soft chewable formulation may be used for preventing a parasitic, insect, acarid, or helminth infestation. In one embodiment, the soft chewable formulation may be used for ameliorating a parasitic, insect, acarid, or helminth infestation. In some embodiments, the soft chewable formulation may be used for controlling a parasitic, insect, acarid, or helminth infestation. In some embodiments, the soft chewable formulation may be used to treat a parasitic nematode infestation. The term “controlling a parasitic insect- and acarid infestation” refers to preventing, reducing or eliminating an infestation by such parasites on animals preferably by killing the insects and/or acarids or nematode parasites within hours or days.

In some embodiments, the subject is administered an effective amount of a soft chewable formulation as described herein to treat, prevent, control, or ameliorate ectoparasites. Ectoparasites is one or more insects or arachnids including those of the genera Ctenocephalides, Rhipicephalus, Dermacentor, Ixodes, Boophilus, Ambylomma, Haemaphysalis, Hyalomma, Sarcoptes, Psoroptes, Otodectes, Chorioptes, Hypoderma, Gasterophilus, Lucilia, Dermatobia, Cochliomyia, Chrysomyia, Damalinia, Linognathus, Haematopinus, Solenopotes, Trichodectes, and Felicola. In some embodiments for the treatment against ectoparasites, the ectoparasite is from the genera Ctenocephalides, Rhipicephalus, Dermacentor, Ixodes and/or Boophilus. The ectoparasites treated include but are not limited to fleas, ticks, mites, mosquitoes, flies, lice, blowfly and combinations thereof. Specific examples include, but are not limited to, cat and dog fleas (Ctenocephalides felis, Ctenocephalides sp. and the like), ticks (Rhipicephalus sp., Ixodes sp., Dermacentor sp., Amblyomma sp., Haemaphysalis sp., and the like), and mites (Demodex sp., Sarcoptes sp., Otodectes sp., Cheyletiella sp., and the like), lice (Trichodectes sp., Felicola sp., Linognathus sp., and the like), mosquitoes (Aedes sp., Culex sp., Anopheles sp., and the like) and flies (Hematobia sp. including Haematobia irritans, Musca sp., Stomoxys sp. including Stomoxys calcitrans, Dermatobia sp., Cochliomyia sp., and the like).

Additional examples of ectoparasites include but are not limited to the tick genus Boophilus, especially those of the species microplus (cattle tick), decoloratus and annulates, myiases such as Dermatobia hominis (known as Berne in Brazil) and Cochliomyia hominivorax (greenbottle); sheep myiases such as Lucilia sericata, Lucilia cuprina (known as blowfly strike in Australia, New Zealand and South Africa) and Gasterophilus in horses. Flies proper, namely those whose adult constitutes the parasite, such as Haematobia irritans (horn fly) and Stomoxys calcitrans (stable fly); lice such as Linognathus vituli, etc.; and mites such as Sarcoptes scabiei and Psoroptes ovis. The above list is not exhaustive and other ectoparasites are known to be harmful to animals and humans. These include, for example migrating dipteran larvae.

In some embodiments, the subject is administered an effective amount of a soft chewable formulation as described herein to treat, prevent, control, or ameliorate helminths. Helminths may be selected from the group comprising Anaplocephala, Ancylostoma, Anecator, Ascaris, Capillaria, Cooperia, Cyathostomum, Dipylidium, Dirofilaria, Echinococcus, Enterobius, Fasciola, Haemonchus, Oesophagostumum, Ostertagia, Parascaris, Toxocara, Strongylus, Strongyloides, Toxascaris, Trichinella, Trichuris and Trichostrongylus, among others.

In some embodiments, the subject is administered an effective amount of a soft chewable formulation as described herein to treat of at least about 90% against roundworm (Toxocara canis), whipworm (Trichuris vulpis) or hookworm (Ancylostoma caninum) while also controlling ectoparasites (e.g., fleas and ticks) with a high level of efficacy, as described above. In some embodiments, the subject is administered an effective amount of a soft chewable formulation as described herein to treat of at least 95% against roundworm (Toxocara canis), whipworm (Trichuris vulpis) or hookworm (Ancylostoma caninum). In some embodiments, the subject is administered an effective amount of a soft chewable formulation as described herein to treat of at least about 100% against Dirofilaria immitis (heartworm).

In some embodiments, the subject is administered an effective amount of a soft chewable formulation as described herein to treat, prevent, control, or ameliorate ticks. Ticks include, but are not limited to Dermacentor variabilis, Ixodes scapularis, Amblyomma americanum, Rhipicephalus sanguineus, Ixodes ricinus, Dermacentor reticulatus and Ixodes holocyclus.

In some embodiments, the subject is administered an effective amount of a soft chewable formulation as described herein to treat, prevent, control, or ameliorate inflammation. In some embodiments, the inflammation is acute inflammation. In some embodiments, the inflammation is chronic inflammation. In some embodiments, the inflammation is toxic inflammation. In some embodiments, the inflammation is infectious inflammation. In some embodiments, the inflammation is associated with a specific disease or condition selected from, but not limited to, arthritis, mastitis, encephalitis, dermatitis, bronchitis, cystitis, pancreatitis, colitis, otitis, or a combination thereof. In some embodiments, the subject is administered a therapeutically effective amount in a single dose. In some embodiments, the subject is administered a therapeutically effective amount in multiple doses.

In some embodiments, the subject is administered an effective amount of a soft chewable formulation as described herein to treat, prevent, control, or ameliorate a behavioral condition. In some embodiments, the behavioral condition is general anxiety. In some embodiments, the behavioral condition is separation anxiety. In some embodiments, the behavioral condition is aggression. In some embodiments, the behavioral condition is fear-based aggression. In some embodiments, the behavioral condition is a compulsive disorder. In some embodiments, the behavioral condition is a cognitive dysfunction syndrome (CDS). In some embodiments, the behavioral condition is a urine-marking or inappropriate elimination condition or disorder. In some embodiments, the behavioral condition is noise phobias. In some embodiments, the behavioral condition is head-pressing or head-sharking in horses.

In some embodiments, the subject is administered an effective amount of a soft chewable formulation as described herein to treat, prevent, control, or ameliorate a gastrointestinal disease or condition. In some embodiments, the gastrointestinal disease or condition is vomiting. In some embodiments, the gastrointestinal disease or condition is diarrhea. In some embodiments, the gastrointestinal disease or condition is a stomach ulcer.

In some embodiments, the subject is administered an effective amount of a soft chewable formulation as described herein to treat, prevent, control, or ameliorate hypertension. In some embodiments, the subject is administered an effective amount of a soft chewable formulation as described herein to treat, prevent, control, or ameliorate hypotension. In some embodiments, the subject is administered an effective amount of a soft chewable formulation as described herein to treat, prevent, control, or ameliorate hyperthyroidism. In some embodiments, the subject is administered an effective amount of a soft chewable formulation as described herein to treat, prevent, control, or ameliorate hypothyroidism. In some embodiments, the subject is administered an effective amount of a soft chewable formulation as described herein to treat, prevent, control, or ameliorate seizures.

In some embodiments, the soft chewable formulation may be provided at an effective amount of the active ingredients, meaning a non-toxic, but sufficient amount to provide the desired control effect. A person skilled in the art using routine experimentation may determine an appropriate “effective” amount in any individual case. Such an amount will depend on the age, condition, weight and type of the target animal. The soft chews may be formulated to contain an amount of active ingredients that is adjusted to animals in a specific weight range. The animals may receive a dosage every 2, 3, 4, 5 or 6 months or receives a monthly, weekly or daily dosage. The treatment can, for example, be continuing or seasonal.

In some embodiments, the soft chewable formulation as described herein may be administered to all species of animals that have an infestation. The recipient of the product may be a livestock animal, e.g., sheep, cattle, pig, goat or poultry; a laboratory test animal, e.g., guinea pig, rat or mouse; or a companion animal, e.g., dog, cat, rabbit, ferret or horse. The product according to the invention is especially suitable for use in companion animals, e.g., dogs, cats or ferrets. In some embodiments, the soft chewable formulation may be administered to warm-blooded animals, such as humans, cattle, sheep, pigs, cats, dogs, horses, llamas, deer, rabbits, skunks, raccoons, camels, etc., or birds. In some embodiments, the soft chewable may be administered to dogs, cats, horses, and other companion animals.

In some embodiments, the subject is administered an effective amount of a soft chewable formulation as described herein providing protection of at least 90% efficacy against one or more infections for at least 30 days. In some embodiments, the subject is administered an effective amount of a soft chewable formulation as described herein providing protection of at least 90% efficacy against one or more infection for at least 45 days. In some embodiments, the subject is administered an effective amount of a soft chewable formulation as described herein providing protection of at least 90% efficacy against one or more infections for at least 60 days. In some embodiments, the subject is administered an effective amount of a soft chewable formulation as described herein providing protection of at least 95% efficacy against one or more infections for at least 30 days. In some embodiments, the subject is administered an effective amount of a soft chewable formulation as described herein providing protection of at least 95% efficacy against one or more infections for at least 45 days. In some embodiments, the subject is administered an effective amount of a soft chewable formulation as described herein providing protection of at least 95% efficacy against one or more infections for at least 60 days.

In some embodiments, the subject is administered an effective amount of a soft chewable formulation as described herein treats at least 90% of at least one or more infections after 5 days. In some embodiments, the subject is administered an effective amount of a soft chewable formulation as described herein treats at least 95% of at least one or more infections after 5 days. In some embodiments, the subject is administered an effective amount of a soft chewable formulation as described herein treats at least 99% of at least one or more infections after 5 days. In some embodiments, the subject is administered an effective amount of a soft chewable formulation as described herein treats at least 90% of at least one or more infections after 10 days. In some embodiments, the subject is administered an effective amount of a soft chewable formulation as described herein treats at least 95% of at least one or more infections after 10 days. In some embodiments, the subject is administered an effective amount of a soft chewable formulation as described herein treats at least 99% of at least one or more infections after 10 days.

In some embodiments, the subject is administered an effective amount of a soft chewable formulation as described herein providing control of at least 90% of one or more infections for at least 30 days. In some embodiments, the subject is administered an effective amount of a soft chewable formulation as described herein providing control of at least 90% of one or more infections for at least 45 days. In some embodiments, the subject is administered an effective amount of a soft chewable formulation as described herein providing control of at least 90% of one or more infections for at least 60 days. In some embodiments, the subject is administered an effective amount of a soft chewable formulation as described herein providing control of at least 95% of one or more infections for at least 30 days. In some embodiments, the subject is administered an effective amount of a soft chewable formulation as described herein providing control of at least 95% of one or more infections for at least 45 days. In some embodiments, the subject is administered an effective amount of a soft chewable formulation as described herein providing control of at least 95% of one or more infections for at least 60 days.

In some embodiments, a subject receives sufficient active ingredient from the soft chewable formulation from multiple dosages before sufficient levels of the active ingredient are achieved. One can readily and immediately envision a regimen wherein a subject is administered a first soft chewable formulation, and the subject receives one or more subsequent soft chewable formulation dosages. Such a regimen may continue such that the subject receives a soft chewable formulation dosage after the subject receives the second composition dosage. In some embodiments, a subject may receive: 1, 2, 3, 4, 5, 6, 7, 8, 9, 10 or more doses during treatment.

In some instances, a period of time passes between administering soft chewable formulation doses to a subject. In some embodiments, the time period between soft chewable formulation dosages is equal to or at least about: 1 day, 1 week, 2 weeks, 3 weeks, 1 month, 2 months, 3 months, 4 months, 5 months, 6 months, 7 months, 8 months, 9 months, 10 months, 11 months, 1 year, or ranges including and/or spanning the aforementioned values. In some embodiments, one or more additional soft chewable formulation or therapeutic agents are administered to the subject during the period between the subject's administrations of the soft chewable formulation.

In aspects, administering a composition described herein may increase longevity, survival time, life span, or health span of the subject. In some embodiments, the expected longevity, survival time, life span, or health span of the subject is the median expectation for similarly situated subjects. In other embodiments, the expected longevity, survival time, life span, or health span of the subject is the mean expectation for similarly situated subjects. Subjects of similar situation may be determined based upon any one or more factors, including but not limited to, age, health, family history, or activity levels. In some embodiments, the expected increase as measured from the time treatment is started may be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 1000%, about any of the aforementioned percentages, or a range bounded by any of the aforementioned percentages (e.g., about 1%-30%, about 5%-25%, about 5%-20%, about 5%-15% or 1%-30%, 5%-25%, 5%-20%, 5%-15%), 1%-100%, 1%-90%, 1%-80%, 1%-70%, 1%-60%, 1%-50%, 1%-40%, 1%-30%, 1%-20%, 1%-10%, 10%-100%, 10%-90%, 10%-80%, 10%-70%, 10%-70%, 10%-60%, 10%-50%, 10%-40%, 10%-30%, 10%-20%, 20%-100%, 20%-90%, 20%-80%, 20%-70%, 20%-60%, 20%-50%, 20%-40%, 20%-30%, 30%-100%, 30%-90%, 30%-80%, 30%-70%, 30%-60%, 30%-50%, 30%-40%, 40%-100%, 40%-90%, 40%-80%, 40%-70%, 40%-60%, 40%-50%, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-1000%, about any of the aforementioned range of percentages (e.g., about 10%-70%, about 30%-60%, or about 50%-70%), relative to the expected longevity, survival time, life span, or health span of the subject. In some embodiments, the expected increase is in years and is 1-40 years, 1-19 years, 1-18 years, 1-17 years, 1-16 years, 1-15 years, 1-14 years, 1-13 years, 1-12 years, 1-11 years, 1-10 years, 1-9 years, 1-8 years, 1-7 years, 1-6 years, 1-5 years, 1-4 years, 1-3 years, 1-2 years, 1 year, at least the aforementioned years (e.g., at least 1-10 years), or about the aforementioned years (e.g., about 1-2 years or at least about 1-2 years), relative to the expected longevity, survival time, life span, or health span of the subject. In some embodiments, the expected increase is in days to months, and is one day to one year, one day to 11 months, one day to 10 months, one day to 9 months, one day to 8 months, one day to 7 months, one day to 6 months, one day to 5 months, one day to 4 months, one day to 3 months, one day to 2 months, one day to one month, at least the aforementioned range of days to months (e.g., at least one day to 11 months), or about the aforementioned range of days to months (e.g., about one day to 6 months or at least about one day to 6 months), relative to the expected longevity, survival time, life span, or health span of the subject.

In some embodiments, the soft chewable formulation as described herein are made to be appetizing to an animal (e.g., by including a flavorant that tends to induce an animal to chew upon it), thereby inducing the animal to consume an otherwise undesirable one or more active ingredients. Such formulations may result in an animal masticating the soft chew formulation sufficiently to dissolve the entire soft chew formulation. In some embodiments, the soft chewable formulation is appetizing to an animal with a sufficient palatable property to ensure voluntary uptake. In some embodiments, the soft chewable formulation provides a palatable dosage form to entice an animal to consume it. In some embodiments, the soft chewable formulation provides a palatable dosage form for voluntary acceptance of the soft chewable formulation. In some embodiments, the soft chewable formulation provides a palatable dosage form for spontaneous consumption by an animal. In some embodiments, the soft chewable formulation provides a palatable form according to all standards promulgated by the Committee for Medicinal Products for Veterinary Use (CVMP) Guidance. In some embodiments, the soft chew formulation is formulated so that the animal may chew on the soft chew formulation for at least about 2.5 seconds before being swallowed. In some embodiments, the soft chew formulation is formulated so that the animal may chew on the soft chew formulation for at least about 5 seconds before being swallowed. In some embodiments, the soft chew formulation is formulated so that the animal may chew on the soft chew formulation for at least about 10 seconds before being swallowed. In some embodiments, the soft chew formulation is formulated so that the animal may chew on the soft chew formulation for at least about 15 seconds before being swallowed. In some embodiments, the soft chew formulation is formulated so that the animal may chew on the soft chew formulation for at least about 20 seconds before being swallowed. In some embodiments, the soft chew formulation is formulated so that the animal chews on the soft chew formulation for less than or equal to about 2.5 seconds before being swallowed. In some embodiments, the soft chew formulation is formulated so that the animal chews on the soft chew formulation for less than or equal to about 5.0 seconds before being swallowed. In some embodiments, the soft chew formulation is formulated so that the animal chews on the soft chew formulation for less than or equal to about 7.5 seconds before being swallowed. In some embodiments, the soft chew formulation is formulated so that the animal chews on the soft chew formulation for less than or equal to about 10 seconds before being swallowed. In some embodiments, the soft chew formulation is formulated so that the animal chews on the soft chew formulation for less than or equal to about 15 seconds before being swallowed. In some embodiments, the soft chew formulation is formulated so that the animal chews on the soft chew formulation for less than or equal to about 20 seconds before being swallowed. In some embodiments, the soft chew formulation is formulated so that the animal swallows intact the soft chew formulation. In some embodiments, the soft chew formulation is formulated so that the animal swallows an at least about 95% intact soft chew formulation. In some embodiments, the soft chew formulation is formulated so that the animal swallows an at least about 90% intact soft chew formulation. In some embodiments, the soft chew formulation is formulated so that the animal swallows an at least about 85% intact soft chew formulation. In some embodiments, the soft chew formulation is formulated so that the animal swallows an at least about 80% intact the soft chew formulation. In some embodiments, the soft chew formulation is formulated so that the animal swallows an at least about 75% intact soft chew formulation. In some embodiments, the soft chew formulation is formulated so that the animal swallows an at least about 70% intact soft chew formulation. In some embodiments, the soft chew formulation is formulated so that the animal swallows an intact formulation after holding the soft chew formulation in its mouth for less than or equal to 2.5 seconds. In some embodiments, the soft chew formulation is formulated so that the animal swallows an intact formulation after holding the soft chew formulation in its mouth for less than or equal to 5.0 seconds. In some embodiments, the soft chew formulation is formulated so that the animal swallows an intact formulation after holding the soft chew formulation in its mouth for less than or equal to 7.5 seconds.

Accordingly, some aspects described relate to the following numbered alternatives:

1. A soft chewable veterinary formulation, the formulation comprising: a starch between about 2% w/w and about 50% w/w; one or more carbohydrates between about 5% w/w and about 25% w/w; a flavoring agent between about 5% w/w and about 15% w/w; a disintegrant between about 0.1% w/w and about 10.0% w/w; a lipid between about 5% w/w and about 15% w/w; and at least one active ingredient between about 0.001% w/w and about 20% w/w, wherein, the starch comprises a regular starch and a pre-gelatinized starch that is in a ratio from about 3:1 to about 15:1.

2. The soft chewable veterinary formulation of alternative 1, wherein the flavoring agent is an animal-derived flavoring agent.

3. The soft chewable veterinary formulation of alternative 2, wherein the animal-derived flavoring agent is chicken liver powder, pork liver powder, beef liver powder, ham, fish, or a combination thereof.

4. The soft chewable veterinary formulation of alternative 3, wherein the chicken-liver comprises about 8% to about 14% of the soft chewable veterinary formulation.

5. The soft chewable veterinary formulation of any one of alternatives 1 to 4, wherein the soft chewable veterinary formulation further comprises a humectant.

6. The soft chewable veterinary formulation of alternative 5, wherein the humectant is glycerin, glycerol triacetate, polydextrose, lactic acid, or a combination thereof.

7. The soft chewable veterinary formulation of alternative 5 or 6, wherein the humectant comprises between about 8% w/w and about 20% w/w of the soft chewable veterinary formulation.

8. The soft chewable veterinary formulation of any one of alternatives 1 to 7, wherein the soft chewable veterinary formulation further comprises a preservative.

9. The soft chewable veterinary formulation of alternative 8, wherein the preservative is benzalkonium chloride, benzethonium chloride, benzoic acid, benzyl alcohol, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, propyl paraben, myristyl gamma-picolinium chloride, methylparaben, propylparaben, quaternary ammonium compounds, or combinations thereof.

10. The soft chewable veterinary formulation of alternative 8 or 9, wherein the preservative comprises between about 0.05% w/w to about 3.0% w/w of the soft chewable veterinary formulation.

11. The soft chewable veterinary formulation of any one of alternatives 1 to 10, wherein the soft chewable veterinary formulation further comprises a binder.

12. The soft chewable veterinary formulation of alternative 11, wherein the binder comprises between about 0.1% to about 5.0% w/w.

13. The soft chewable veterinary formulation of any one of alternatives 1 to 12, wherein the disintegrant is sodium starch glycolate, crospovidone, croscarmellose sodium, microcrystalline cellulose, alginic acid, veegum, bentonite, croscarmellose, or combinations thereof.

14. The soft chewable veterinary formulation of alternative 13, wherein the disintegrant comprises between about 0.1% w/w to about 5.0% w/w of the soft chewable veterinary formulation.

15. The soft chewable veterinary formulation of any one of alternatives 1 to 14, wherein the soft chewable veterinary formulation further comprises an emulsifying agent.

16. The soft chewable veterinary formulation of alternative 14, wherein the emulsifying agent is potassium laurate, triethanolamine stearate, sodium lauryl sulfate, alkyl polyoxyethylene sulfates, sodium dodecyl sulfate, dioctyl sodium sulfosuccinate, polyoxyethylene fatty acid derivatives of the sorbitan esters, polyoxyethylene fatty alcohol ethers sorbitan fatty acid esters polyoxyethylene alkyl ethers, polyoxyethylene sorbitan fatty acid esters, polyoxyethylene polyoxypropylene block copolymers, polyethylene glycol 400 monostearate, lanolin alcohols, hydrophilic colloids, alginates, acacia, tragacanth, xanthan, vitamin E, pectin, gelatin, casein, lecithin, or combinations thereof.

17. The soft chewable veterinary formulation of alternative 15 or 16, wherein the emulsifying agent is less than or equal to about 5.0% w/w of the soft chewable veterinary formulation.

18. The soft chewable veterinary formulation of any one of alternatives 1 to 17, wherein the at least one active ingredient is a moisture-sensitive active ingredient.

19. The soft chewable veterinary formulation of any one of alternatives 1 to 17, wherein the at least one active ingredient is at least one active anti-parasitic active ingredient.

20. The soft chewable veterinary formulation of alternative 19, wherein the at least one active ingredient is selected from the group consisting of pyrantel pamoate, ivermectin, pimobendan, firocoxib, carprofen, maropitant citrate, and praziquantel.

21. The soft chewable veterinary formulation of any one of alternatives 1 to 20, wherein the lipid is a vegetable oil.

22. The soft chewable veterinary formulation of alternative 21, wherein the vegetable oil is refined soybean oil.

23. The soft chewable veterinary formulation of any one of alternatives 1 to 22, wherein the one or more carbohydrates is a simple carbohydrate.

24. The soft chewable veterinary formulation of alternative 23, wherein the simple carbohydrate is dextrose, ribose, fructose, sucrose, maltose, erythritol, xylitol, hydrogenatetd isomaltulose, maltitol, or combinations thereof.

25. The soft chewable veterinary formulation of any one of alternatives 1 to 23, wherein the soft chewable veterinary formulation further comprises a sweetener.

26. The soft chewable veterinary formulation of alternative 25, wherein the sweetener is honey, maple syrup, corn syrup, synthetic sweeteners, natural sweeteners, or combinations thereof.

27. The soft chewable veterinary formulation of alternative 25 or 26, wherein the sweetener is between about 1% to about 10% of the soft chewable veterinary formulation.

28. The soft chewable veterinary formulation of any one of alternatives 1 to 27, wherein the soft chewable veterinary formulation further comprises a coloring agent.

29. The soft chewable veterinary formulation of alternative 28, wherein the coloring agent is caramel color liquid.

30. The soft chewable veterinary formulation of alternative 28 or 29, wherein the coloring agent is between about 0.1% to about 4.0% of the soft chewable veterinary formulation.

31. The soft chewable veterinary formulation of any one of alternatives 1 to 30, wherein the chewable veterinary formulation further comprises one or more components selected from the group consisting of surfactants, wetting agents, pH stabilizers, a softening agent, and a solvent.

32. The soft chewable veterinary formulation of any one of alternatives 1 to 31, wherein the soft chewable veterinary formulation has added water content of less than about 5%.

33. The soft chewable veterinary formulation of any one of alternatives 1 to 31, wherein the soft chewable veterinary formulation has a moisture content less than about 10%.

34. The soft chewable veterinary formulation of any one of alternatives 1 to 33, wherein the at least one active ingredient is a nutrient.

35. A soft chewable veterinary formulation, the formulation comprising: a liquid between about 20% to about 40%; a regular starch between about 20% to about 50%; a pre-gelatinized starch between about 2% to about 50%, and an active ingredient between about 0.01% and about 15%, wherein, the total of the active ingredient and the regular starch comprises about 35% to about 50% of the soft chewable veterinary formulation, wherein, the ratio of the active ingredient and the regular starch to the pre-gelatinized starch is in a ratio from about 3:1 to about 15:1.

36. A method of treating, controlling, preventing, or ameliorating a disease or condition in a subject in need thereof, the method comprising: administering to the subject a soft chewable formulation of any one of alternatives 1 to 34, thereby, treating, controlling, preventing, or ameliorating the disease or condition in the subject in need thereof.

37. The method of alternative 36, wherein the disease or condition is associated with a viral infection.

38. The method of alternative 37, wherein the viral infection is selected from canine distemper, canine influenza, canine parvovirus, herpes, rabies, feline calicivirus, coronavirus, feline leukemia virus, feline panleukopenia virus, feline calicivirus, feline herpes virus, varicella zoster virus, herpes simplex virus, cytomegalovirus, or Epstein-Barr virus.

39. The method of alternative 36, wherein the disease or condition is associated with a bacterial infection or condition.

40. The method of alternative 39, wherein the bacterial infection or condition is selected from Bacillus anthracia, Bacillus cereus, Bartonella henselae, Bartonella quintana, Bordetella pertussis, Borrelia burgdorferi, Borrelia garinii, Borrelia afzelhi, Borrelia recurrentis, Brucella abortus, Brucella canis, Brucella melitensis, Brucella suis, Campylobacter jejuni, Chlamydia pneumonia, Chlamydia trachomatis, Chlamydophila psittaci, Clostridium botulinum, Clostridium difficile, Clostridium perfringens, Clostridium tetani, Corynebacterium diphtheria, Enterococcus faecalis, Enierococcus faecium, Escherichia coli, Francisella tularensis, Haemophilus influenza, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumonia, Neisseria gonorrhoeae, Neisseria meningitides, Pseudomonas aeruginosa, Rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumonia, Streptococcus pyogenes, Treponema pallidum, Ureaplasma urealyticum, Vibrio cholera, Yersinia pestis, Yersinia enterocolitica, Yersinia pseudotuberculosis.

41. The method of alternative 36, wherein the disease or condition is associated with a fungus disease or condition.

42. The method of alternative 41, wherein the fungal disease or condition is selected from Histoplasma capsulatum (causes Histoplasmosis), Blastomyces dermatitidis (causes Blastomycosis), and Coccidioides immitis (causes Coccidioidomycosis (Valley Fever)), Aspergillosis fumigaus (causes Aspergillosis), Aspergillus tereus (causes Disseminated Aspergillosis), Aspergillus deflectus, Aspergillus niger, Candida albicans (causes Candidiasis), Cryptococcus neoformans (causes Cryptococcosis), Cryptococcus gattii, Geotrichum candidum (causes Geotrichosis), Pythium insidiosum (causes Oomycosis also called pythiosis), Rhinosporidium seeberi (causes Rhinosporidiosis), and Sporothrix schenckii (causes Sporotrichosis).

43. A method of treating, controlling, preventing, or ameliorating a parasitic, insect, acarid, or helminth infestation in a subject in need thereof, the method comprising: administering to the subject a soft chewable formulation of any one of alternatives 1 to 34, thereby, treating, controlling, preventing, or ameliorating the parasitic, insect, acarid, or helminth infestation in a subject in need thereof.

44. A method of treating, controlling, preventing, or ameliorating an infection in a subject in need thereof, the method comprising: administering to the subject a soft chewable formulation of any one of alternatives 1 to 34, thereby, treating, controlling, preventing, or ameliorating the infection in a subject in need thereof.

45. The method of alternative 43, wherein treating the parasitic, insect, acarid, or helminth infestation reduces the parasitic, insect, acarid, or helminth infestation by at least about 90% after about 5 days.

46. The method of alternative 44, wherein treating the infection reduces infection by at least about 90% after about 5 days.

47. The method of any one of alternatives 36 to 46, wherein the soft chewable formulation is administered to the subject at least once a day.

48. The method of any one of alternatives 36 to 46, wherein the soft chewable formulation is administered to the subject once a week.

49. The method of any one of alternatives 36 to 46, wherein the soft chewable formulation is administered to the subject once a month.

50. The method of any one of alternatives 36 to 46, wherein the soft chewable formulation is administered to the subject once every 3 months.

51. The method of any one of alternatives 36 to 50, wherein the subject is a companion animal.

52. The method of alternative 51, wherein the companion animal is a cat, dog, or horse.

53. A method of treating, controlling, preventing, managing, or ameliorating pain in a subject in need thereof, the method comprising: administering to the subject a soft chewable formulation of any one of alternatives 1 to 34, thereby, treating, controlling, preventing, or ameliorating the pain in a subject in need thereof.

54. A method of treating, controlling, preventing, managing, or ameliorating heart disease in a subject in need thereof, the method comprising: administering to the subject a soft chewable formulation of any one of alternatives 1 to 34, thereby, treating, controlling, preventing, or ameliorating the chronic heart disease in a subject in need thereof.

55. A method of treating, controlling, preventing, managing, or ameliorating nausea and vomiting in a subject in need thereof, the method comprising: administering to the subject a soft chewable formulation of any one of alternatives 1 to 34, thereby, treating, controlling, preventing, or ameliorating the nausea and vomiting in a subject in need thereof.

56. A method of treating, controlling, preventing, managing, or ameliorating inflammation in a subject in need thereof, the method comprising: administering to the subject a soft chewable formulation of any one of alternatives 1 to 34, thereby, treating, controlling, preventing, or ameliorating the nausea and vomiting in a subject in need thereof.

57. A method of treating, controlling, preventing, managing, or ameliorating a gastrointestinal disease in a subject in need thereof, the method comprising: administering to the subject a soft chewable formulation of any one of alternatives 1 to 34, thereby, treating, controlling, preventing, or ameliorating the gastrointestinal disease subject in need thereof.

58. A method of treating, controlling, preventing, managing, or ameliorating a behavioral conditional in a subject in need thereof, the method comprising: administering to the subject a soft chewable formulation of any one of alternatives 1 to 34, thereby, treating, controlling, preventing, or ameliorating the behavioral condition in a subject in need thereof.

59. A method of treating, controlling, preventing, managing, or ameliorating hypertension in a subject in need thereof, the method comprising: administering to the subject a soft chewable formulation of any one of alternatives 1 to 34, thereby, treating, controlling, preventing, or ameliorating the hypertension in a subject in need thereof.

60. An edible pellet with one or more active agents, the edible pellet being configured to be consumed by a predetermined type of animal, the edible pellet comprising: a matrix comprising an outer surface with a predefined shape that is sufficiently hard so as to retain the predefined shape during packaging, shipping, storage, and pre-consumption handling without crumbling, flaking, or fracturing, the matrix comprising one or more active agents; wherein the matrix is chewable such that upon initial mastication in the mouth of the animal, applying a typical mastication pressure by animals of this type, the hard outer surface of the matrix disintegrates and the matrix transforms into a soft grainy amalgam; and wherein the edible matrix retains its hardness and chewability properties for a shelf-life of at least two years.

61. The edible pellet of alternative 60, wherein the one or more active agents is uniformly dispersed within the matrix.

62. The edible pellet of alternative 60 or 61, wherein the matrix further comprises a regular starch and a pre-gelatinized starch in a ratio from about 3:1 to about 15:1.

63. The edible pellet of alternative 62, wherein the matrix further comprises at least one of one or more carbohydrates, a flavoring agent, a disintegrant, a lipid, a flavoring agent, a humectant, a preservative, a binder, an emulsifying agent, a sweetener, a coloring agent, surfactants, wetting agents, pH stabilizers, a softening agent, and a solvent.

64. The edible pellet of any one of alternatives 60 to 63, wherein the edible pellet will maintain is predefined shape after 200 newtons of force is applied to the edible pellet.

65. The edible pellet of any one of alternatives 60 to 64, wherein the soft grainy amalgam induces the animal to chew thereupon.

66. The edible pellet ofany one of alternatives 60 to 65, wherein the matrix exhibits sufficient friability upon mastication in the mouth of the animal that the edible pellet is consumed by the animal in not more than 5 seconds of chewing time.

67. The edible pellet of any one of alternatives 60 to 66, wherein the amalgam has a particle or granular material size from 50 microns to 1000 microns.

68. The edible pellet of any one of alternatives 60 to 67, wherein the edible matrix retains its moldable properties for at three months.

### EXAMPLES

### Example 1—Soft Chewable Formulation

This example provides a soft chewable formulation of an embodiment of the disclosure as described in Table 1.

TABLE 1

| Raw Material Name | Percentage (%) w/w |
|---|---|
| Regular Starch | 33 |
| Pre-gelatinized Starch | 5 |
| Chicken Liver Powder | 10 |
| Dextrose | 11 |
| Lecithin Powder | 3 |
| Anthelmintic (Active) | 5.7 |
| Macrocyclic Lactone (Active) | <1 |
| Potassium Sorbate Powder | <1 |
| Vitamin E T-70 (Tocopherol) | <1 |
| Refined Soybean Oil | 7 |
| Glycerin | 11 |
| Caramel Color Liquid | 1.5 |
| Corn Syrup | 4 |
| Sorbitol | 3.5 |
| Crospovidone | 4 |
| Croscarmellose | <1 |
| Total Liquid | 27.1 |
| Total Starch Ratio (Regular Starch/Pre- | (33:5) |
| gelatinized Starch) | |

It was observed that the dough was soft and could extrude easily.

### Example 2—Soft Chewable Formulation

This example provides a soft chewable formulation of an embodiment of the disclosure as described in Table 2.

TABLE 2

| Raw Material Name | Percentage (%) w/w |
|---|---|
| Regular Starch | 32 |
| Pre-gelatinized Starch | 5 |
| Chicken Liver Powder | 10 |
| Dextrose | 11 |
| Lecithin Powder | 3 |
| A broad spectrum macrocyclic lactone | 5.7 |
| (Active) | |
| Ivermectin (Active) | <1 |
| Potassium Sorbate Powder | <1 |
| Vitamin E T-70 (Tocopherol) | <1 |
| Refined Soybean Oil | 7 |
| Glycerin | 11 |
| Caramel Color Liquid | 1.5 |
| Corn Syrup | 4 |
| Sorbitol | 4.5 |
| Crospovidone | 4 |
| Croscarmellose | <1 |
| Total Liquid | 28.1 |
| Total Starch Ratio (Regular Starch/Pre- | (32:5) |
| gelatinized Starch) | |

It was observed that the dough was soft and could extrude easily.

### Example 3—Soft Chewable Formulation

This example provides a soft chewable formulation of an embodiment of the disclosure as described in Table 3.

TABLE 3

| Raw Material Name | Percentage (%) w/w |
|---|---|
| Regular Starch | 31 |
| Pre-gelatinized Starch | 5 |
| Chicken Liver Powder | 10 |
| Dextrose | 11 |
| Lecithin Powder | 3 |
| Pyrantel Pamoate (Active) | 5.7 |
| Ivermectin (Active) | <1 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4.5 |
| Crospovidone | 4 |
| Croscarmellose | <1 |
| Total Liquid | 28.6 |
| Total Starch Ratio (Regular Starch/Pre- | (31:5) |
| gelatinized Starch) | |

It was observed that the dough was soft and could extrude easily.

### Example 4—Soft Chewable Formulation

This example provides a soft chewable formulation of an embodiment of the disclosure as described in Table 4.

TABLE 4

| Raw Material Name | Percentage (%) w/w |
|---|---|
| Regular Starch | 34 |
| Pre-gelatinized Starch | 5 |
| Chicken Liver Powder | 10 |
| Dextrose | 13 |
| Lecithin Powder | 3 |
| Anti-parasitic ingredient (Active) | <1 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4.5 |
| Crospovidone | 4 |
| Croscarmellose | <1 |
| Total Liquid | 28.6 |
| Total Starch Ratio (Regular Starch/Pre- | (34:5) |
| gelatinized Starch) | |

It was observed that the dough was soft and could extrude easily.

### Example 5—Soft Chewable Formulation

This example provides a soft chewable formulation of an embodiment of the disclosure as described in Table 5.

TABLE 5

| Raw Material Name | Percentage (%) w/w |
|---|---|
| Regular Starch | 35.5 |
| Pre-gelatinized Starch | 3.5 |
| Chicken Liver Powder | 12 |
| Dextrose | 10 |
| Lecithin Powder | 1 |
| Praziquantel (Active) | <1 |
| Potassium Sorbate Powder | <1 |
| Vitamin E T-70 (Tocopherol) | <1 |
| Refined Soybean Oil | 9.5 |
| Glycerin | 13.5 |
| Corn Syrup | 5 |
| Sorbitol | 4.5 |
| Crospovidone | 3 |
| Croscarmellose | <1 |
| Total Liquid | 32.6 |
| Total Starch Ratio (Regular Starch/Pre- | (35.5:3.5) |
| gelatinized Starch) | |

It was observed that the dough was hard and difficult to extruded. However, it was still able to form a soft chew.

### Example 6—Soft Chewable Formulation

This example provides a soft chewable formulation of an embodiment of the disclosure as described in Table 6.

TABLE 6

| Raw Material Name | Percentage (%) w/w |
|---|---|
| Regular Starch | 35 |
| Pre-gelatinized Starch | 4 |
| Chicken Liver Powder | 12 |
| Dextrose | 10 |
| Lecithin Powder | 1 |
| iosquinoline-pyrazine derivative (Active) | <1 |
| Potassium Sorbate Powder | <1 |
| Vitamin E T-70 (Tocopherol) | <1 |
| Refined Soybean Oil | 9.5 |
| Glycerin | 13.5 |
| Corn Syrup | 5 |
| Sorbitol | 4.5 |
| Crospovidone | 3 |
| Croscarmellose | 0.5 |
| Total Liquid | 32.6 |
| Total Starch Ratio (Regular Starch/Pre- | (35:4) |
| gelatinized Starch) | |

It was observe red that the dough was soft an extruded easily.

### Example 7—Soft Chewable Formulation

This example provides a soft chewable formulation of an embodiment of the disclosure as described in Table 7.

TABLE 7

| Raw Material Name | Percentage (%) w/w |
|---|---|
| Regular Starch | 35 |
| Pre-gelatinized Starch | 4 |
| Chicken Liver Powder | 12 |
| Dextrose | 10 |
| Lecithin Powder | 1 |
| Praziquantel (Active) | <1 |
| Potassium Sorbate Powder | <1 |
| Vitamin E T-70 (Tocopherol) | <1 |
| Refined Soybean Oil | 9 |
| Glycerin | 13 |
| Caramel Color Liquid | 1 |
| Corn Syrup | 4.5 |
| Sorbitol | 4.5 |
| Crospovidone | 3 |
| Croscarmellose | <1 |
| Total Liquid | 32.6 |
| Total Starch Ratio (Regular Starch/Pre- | (35:4) |
| gelatinized Starch) | |

It was observed that the dough was soft and could extrude easily.

### Example 8—Soft Chewable Formulation

This example provides a soft chewable formulation of an embodiment of the disclosure as described in Table 8.

TABLE 8

| Raw Material Name | Percentage (%) w/w |
|---|---|
| Regular Starch | 36 |
| Pre-gelatinized Starch | 4 |
| Chicken Liver Powder | 12 |
| Dextrose | 7.5 |
| Soy Lecithin | 3 |
| Pyrantel Pamoate (Active) | 5.7 |
| Ivermectin (Active) | <1 |
| Potassium Sorbate | <1 |
| Vitamin E 70 (Tocopherol) | <1 |
| Soybean Oil | 9 |
| Glycerin | 12 |
| Caramel Color Liquid | 2 |
| Corn Syrup | 5 |
| Sorbitol | 3 |
| Total Liquid | 31.1 |
| Total Starch Ratio (Regular Starch/Pre- | (36:4) |
| gelatinized Starch) | |

It was observed that the dough was soft an could extrude easily.

### Example 9—Soft Chewable Formulation

This example provides a soft chewable formulation of an embodiment of the disclosure as described in Table 9.

TABLE 9

| Raw Material Name | Percentage (%) w/w |
|---|---|
| Regular Starch | 31 |
| Pre-gelatinized Starch | 4 |
| Chicken Liver Powder | 12 |
| Dextrose | 7.5 |
| Soy Lecithin | 3 |
| Pyrantel Pamoate (Active) | 5.7 |
| Ivermectin (Active) | <1 |
| Potassium Sorbate | <1 |
| Vitamin E 70 (Tocopherol) | <1 |
| Soybean Oil | 9 |
| Glycerin | 12 |
| Caramel Color Liquid | 2 |
| Corn Syrup | 5 |
| Sorbitol | 4.5 |
| Crospovidone | 3 |
| Croscarmellose | <1 |
| Total Liquid | 32.6 |
| Total Starch Ratio (Regular Starch/Pre- | (31:4) |
| gelatinized Starch) | |

It was observed that the dough was little on the dry but could extrude easily.

### Example 10—Soft Chewable Formulation

This example provides a soft chewable formulation of an embodiment of the disclosure as described in Table 10.

TABLE 10

| Raw Material Name | Percentage (%) w/w |
|---|---|
| Regular Starch | 36.5 |
| Pre-gelatinized Starch | 3.5 |
| Chicken Liver Powder | 12 |
| Dextrose | 7.5 |
| Soy Lecithin | 3 |
| Pyrantel Pamoate (Active) | 5.7 |
| Ivermectin (Active) | <1 |
| Potassium Sorbate | <1 |
| Vitamin E 70 (Tocopherol) | <1 |
| Soybean Oil | 9 |
| Glycerin | 12 |
| Caramel Color Liquid | 2 |
| Corn Syrup | 5 |
| Sorbitol | 3 |
| Total Liquid | 31.1 |
| Total Starch Ratio (Regular Starch/Pre- | (36.5:3.5) |
| gelatinized Starch) | |

Dough would appear to be similar to Example 8 and could extrude easily.

### Example 11—Soft Chewable Formulation

This example provides a soft chewable formulation of an embodiment of the disclosure as described in Table 11.

TABLE 11

| Raw Material Name | Percentage (%) w/w |
|---|---|
| Regular Starch | 34 |
| Pre-gelatinized Starch | 4 |
| Chicken Liver Powder | 12 |
| Dextrose | 9.3 |
| Soy Lecithin | 3 |
| Praziquantel (Active) | <1 |
| Potassium Sorbate | <1 |
| Vitamin E 70 (Tocopherol) | <1 |
| Soybean Oil | 9 |
| Glycerin | 12 |
| Caramel Color Liquid | 2 |
| Corn Syrup | 5 |
| Sorbitol | 4.5 |
| Croscarmellose | <1 |
| Crospovidone | 3.0 |
| Total Liquid | 32.6 |
| Total Starch Ratio (Regular Starch/Pre- | (34:4) |
| gelatinized Starch) | |

Dough appeared to be soft and extruded easily.

### Example 12—Soft Chewable Formulation

This example provides a soft chewable formulation of an embodiment of the disclosure as described in Table 12.

TABLE 12

| Raw Material Name | Percentage (%) w/w |
|---|---|
| Regular Starch | 33 |
| Pre-gelatinized Starch | 4 |
| Chicken Liver Powder | 12 |
| Dextrose | 9.3 |
| Soy Lecithin | 3 |
| Praziquantel (Active) | <1 |
| Potassium Sorbate | <1 |
| Vitamin E 70 (Tocopherol) | <1 |
| Soybean Oil | 9 |
| Glycerin | 12 |
| Caramel Color Liquid | 2 |
| Corn Syrup | 5 |
| Sorbitol | 4.5 |
| Croscarmellose | <1 |
| Crospovidone | 3.0 |
| Total Liquid | 32.6 |
| Total Starch Ratio (Regular Starch/Pre- | (33:4) |
| gelatinized Starch) | |

Dough appeared to be soft and extruded easily.

### Example 13—Soft Chewable Formulation

This example provides a soft chewable formulation of an embodiment of the disclosure as described in Table 13.

TABLE 13

| Raw Material Name | Percentage (%) w/w |
|---|---|
| Regular Starch | 37.5 |
| Pre-gelatinized Starch | 3.5 |
| Chicken Liver Powder | 12 |
| Dextrose | 9.3 |
| Soy Lecithin | 3 |
| Praziquantel (Active) | <1 |
| Potassium Sorbate | <1 |
| Vitamin E 70 (Tocopherol) | <1 |
| Soybean Oil | 9 |
| Glycerin | 12 |
| Caramel Color Liquid | 2 |
| Corn Syrup | 5 |
| Sorbitol | 3 |
| Croscarmellose | <1 |
| Primmojel | <1 |
| Total Liquid | 32.6 |
| Total Starch Ratio (Regular Starch/Pre- | (37:4) |
| gelatinized Starch) | |

It was observed that the dough was very soft and would have been difficult to extrude.

### Example 14—Soft Chewable Formulation

This example provides a soft chewable formulation of an embodiment of the disclosure as described in Table 14.

TABLE 14

| Raw Material Name | Percentage (%) w/w |
|---|---|
| Regular Starch | 37.5 |
| Pre-gelatinized Starch | 2.5 |
| Chicken Liver Powder | 12 |
| Dextrose | 7.5 |
| Soy Lecithin | 3 |
| Pyrantel Pamoate (Active) | 5.7 |
| Ivermectin (Active) | <1 |
| Potassium Sorbate | <1 |
| Vitamin E 70 (Tocopherol) | <1 |
| Soybean Oil | 9 |
| Glycerin | 12 |
| Caramel Color Liquid | 2 |
| Corn Syrup | 5 |
| Sorbitol | 3 |
| Total Liquid | 31.1 |
| Total Starch Ratio (Regular Starch/Pre- | (37.5:2.5) |
| gelatinized Starch) | |

Dough would appear to be similar to Example 10 and could extrude easily.

### Example 15—Soft Chewable Formulation

This example provides a soft chewable formulation of an embodiment of the disclosure as described in Table 15.

TABLE 15

| Raw Material Name | Percentage (%) w/w |
|---|---|
| Regular Starch | 35.5 |
| Pre-gelatinized Starch | 4.5 |
| Chicken Liver Powder | 12 |
| Dextrose | 7.5 |
| Soy Lecithin | 3 |
| Pyrantel Pamoate (Active) | 5.7 |
| Ivermectin (Active) | <1 |
| Potassium Sorbate | <1 |
| Vitamin E 70 (Tocopherol) | <1 |
| Soybean Oil | 9 |
| Glycerin | 12 |
| Caramel Color Liquid | 2 |
| Corn Syrup | 5 |
| Sorbitol | 3 |
| Total Liquid | 31.1 |
| Total Starch Ratio (Regular Starch/Pre- | (35.5:4.5) |
| gelatinized Starch) | |

It was observed that the dough was soft and could extrude easily.

### Example 16—Soft Chewable Formulation

This example provides a soft chewable formulation of an embodiment of the disclosure as described in Table 16.

TABLE 16

| Raw Material Name | Percentage (%) w/w |
|---|---|
| Regular Starch | 39 |
| Pregelatinized Starch | 4 |
| Glycerin | 12 |
| Chicken Liver Powder | 12 |
| Dextrose | 9.3 |
| Soy Oil | 9 |
| Lecithin | 3 |
| Praziquantel (Active) | <1 |
| Potassium Sorbate | <1 |
| Tocopherol (Vitamin E T-70) | <1 |
| Caramel Color liquid | 2 |
| Corn Syrup | 5 |
| Sorbitol | 3 |
| Total Liquid | 31.1 |
| Total Starch Ratio (Regular Starch/Pre- | (39:4) |
| gelatinized Starch) | |

Doug appeared soft and could extrude easily.

### Example 17—Soft Chewable Formulation

This example provides a soft chewable formulation of an embodiment of the disclosure as described in Table 17.

TABLE 17

| Raw Material Name | Percentage (%) w/w |
|---|---|
| Regular Starch | 38.5 |
| Pregelatinized Starch | 4.5 |
| Glycerin | 12 |
| Chicken Liver Powder | 12 |
| Dextrose | 9.3 |
| Soy Oil | 9 |
| Lecithin | 3 |
| Praziquantel (Active) | <1 |
| Potassium Sorbate | <1 |
| Vitamin E 70 (Tocopherol) | <1 |
| Caramel Color liquid | 2 |
| Corn Syrup | 5 |
| Sorbitol | 3 |
| Total Liquid | 31.1 |
| Total Starch Ratio (Regular Starch/Pre- | (38.5:4.5) |
| gelatinized Starch) | |

Dough would appear to be similar to Example 16 and could extrude easily.

### Example 18—Soft Chewable Formulation

This example provides a soft chewable formulation of an embodiment of the disclosure as described in Table 18.

TABLE 18

| Raw Material Name | Percentage (%) w/w |
|---|---|
| Regular Starch | 27.5 |
| Pregelatinized Starch | 7 |
| Glycerin | 15 |
| Chicken Liver Powder | 12 |
| Dextrose | 23.3 |
| Soy Oil | 8 |
| Lecithin | 4 |
| Praziquantel (Active) | <1 |
| Potassium Sorbate | <1 |
| Vitamin E 70 (Tocopherol) | <1 |
| Caramel Color | 1.5 |
| Total Liquid | 24.6 |
| Total Starch Ratio (Regular Starch/Pre- | (27.5:7) |
| gelatinized Starch) | |

Dough appeared to be soft and easily extruded.

### Example 19—Soft Chewable Formulation

This example provides a soft chewable formulation of an embodiment of the disclosure as described in Table 19.

TABLE 19

| Raw Material Name | Percentage (%) w/w |
|---|---|
| Regular Starch | 37.5 |
| Pregelatinized Starch | 7 |
| Glycerin | 15 |
| Chicken Liver Powder | 12 |
| Dextrose | 13.3 |
| Soy Oil | 8 |
| Lecithin | 4 |
| Praziquantel (Active) | <1 |
| Potassium Sorbate | <1 |
| Vitamin E 70 (Tocopherol) | <1 |
| Caramel Color | 1.5 |
| Total Liquid | 24.6 |
| Total Starch Ratio (Regular Starch/Pre- | (37.5:7) |
| gelatinized Starch) | |

Dough appeared to be more on the softer side but could be extruded.

### Example 20—Soft Chewable Formulation

This example provides a soft chewable formulation of an embodiment of the disclosure as described in Table 20.

TABLE 20

| Raw Material Name | Percentage (%) w/w |
|---|---|
| Regular Starch | 38.5 |
| Pregelatinized Starch | 7 |
| Glycerin | 15.5 |
| Chicken Liver Powder | 12 |
| Dextrose | 13 |
| Soy Oil | 8 |
| Lecithin | 4 |
| Praziquantel (Active) | <1 |
| Potassium Sorbate | <1 |
| Vitamin E 70 (Tocopherol) | <1 |
| Total Liquid | 23.6 |
| Total Starch Ratio (Regular Starch/Pre- | (38.5:7) |
| gelatinized Starch) | |

Dough appeared to be more on the softer side and would be difficult to extrude.

### Example 21—Soft Chewable Formulation

This example provides a soft chewable formulation of an embodiment of the disclosure as described in Table 21.

TABLE 21

| Raw Material Name | Percentage (%) w/w |
|---|---|
| Pre-gelatinized Starch | 44 |
| Glycerin | 14.5 |
| Chicken Liver Powder | 12 |
| Dextrose | 10 |
| Soy Oil | 8.5 |
| Sorbitol | 6 |
| Lecithin | 3 |
| Praziquantel | <1 |
| Potassium Sorbate | <1 |
| Vitamin E 70 (Tocopherol) | <1 |
| Total Liquid | 29.1 |
| Total Starch Ratio (Regular Starch/Pre- | N/A |
| gelatinized Starch) | |

It was observed that the dough was a little hard to extrude and would have produced chews that would become hard in a few weeks.

### Example 22—Soft Chewable Formulation

This example provides a soft chewable formulation of an embodiment of the disclosure as described in Table 22.

TABLE 22

| Raw Material Name | Percentage (%) w/w |
|---|---|
| Pre-gelatinized Starch | 44 |
| Glycerin | 14.5 |
| Chicken Liver Powder | 12 |
| Dextrose | 10.3 |
| Soy Oil | 8.5 |
| Sorbitol | 6 |
| Lecithin | 3 |
| Praziquantel (Active) | <1 |
| Potassium Sorbate | <1 |
| Vitamin E 70 (Tocopherol) | <1 |
| Total Liquid | 29.1 |
| Total Starch Ratio (Regular Starch/Pre- | N/A |
| gelatinized Starch) | |

It was observed that the dough was a little hard to extrude and would have produced chews that would become hard in a few weeks.

### Example 23—Soft Chewable Formulation

This example provides a soft chewable formulation of an embodiment of the disclosure as described in Table 23.

TABLE 23

| Raw Material Name | Percentage (%) w/w |
|---|---|
| Pre-gelatinized Starch | 35 |
| Glycerin | 20 |
| Chicken Liver Powder | 12 |
| Dextrose | 8.2 |
| Soy Oil | 10 |
| Sorbitol | 7 |
| Regular Starch | 4 |
| Citric Acid | 3 |
| Potassium Sorbate | <1 |
| Propylparaben | <1 |
| Methylparaben | <1 |
| Vitamin E 70 (Tocopherol) | <1 |
| Total Liquid | 37.1 |
| Total Starch Ratio (Regular Starch/Pre- | (4:35) |
| gelatinized Starch) | |

It was observed that the dough was a little hard to extrude and would have produced chews that would become hard in a few weeks.

### Example 24—Soft Chewable Formulation

This example provides a soft chewable formulation of an embodiment of the disclosure as described in Table 24.

TABLE 24

| Raw Material Name | Percentage (%) w/w |
|---|---|
| Pre-gelatinized Starch | 39 |
| Glycerin | 16 |
| Chicken Liver Powder | 12 |
| Dextrose | 8.2 |
| Soy Oil | 9 |
| Sorbitol | 7 |
| Regular Starch | 5 |
| Citric Acid | 3 |
| Potassium Sorbate | <1 |
| Propylparaben | <1 |
| Methylparaben | <1 |
| Vitamin E 70 (Tocopherol) | <1 |
| Total Liquid | 32.1 |
| Total Starch Ratio (Regular Starch/Pre- | (5:39) |
| gelatinized Starch) | |

It was observed that the dough was a little hard to extrude and would have produced chews that would become hard in a few weeks.

### Example 25—Soft Chewable Formulation

This example provides a soft chewable formulation of an embodiment of the disclosure as described in Table 25.

TABLE 25

| Raw Material Name | Percentage (%) w/w |
|---|---|
| Glycerin | 15 |
| Chicken Liver Powder | 12 |
| Dextrose | 11.2 |
| Soy Oil | 8 |
| Sorbitol | 7 |
| Regular Starch | 46 |
| Potassium Sorbate | <1 |
| Propylparaben | <1 |
| Methylparaben | <1 |
| Vitamin E 70 (Tocopherol) | <1 |
| Total Liquid | 30.1 |
| Total Starch Ratio (Regular Starch/Pre- | N/A |
| gelatinized Starch) | |

It was observed that the dough became very hard and could not extrude.

### Example 26—Soft Chewable Formulation

This example provides a soft chewable formulation of an embodiment of the disclosure as described in Table 26.

TABLE 26

| Raw Material Name | Percentage (%) w/w |
|---|---|
| Regular Starch | 44 |
| Glycerin | 13 |
| Gamma Irradiated Chicken | 12 |
| Dextrose | 11.2 |
| Soy Oil | 7 |
| Sorbitol | 7 |
| Lecithin | 5 |
| Potassium Sorbate | <1 |
| Propylparaben | <1 |
| Vitamin E 70 (Tocopherol) | <1 |
| Total Liquid | 27.1 |
| Total Starch Ratio (Regular Starch/Pre- | N/A |
| gelatinized Starch) | |

It was observed that the dough looked soft initially. Extrusion was followed after production. At the start of extrusion, the chews were good but with time the dough became hard and difficult to extrude.

### Example 27—Soft Chewable Formulation

This example provides a soft chewable formulation of an embodiment of the disclosure as described in Table 27.

TABLE 27

| Raw Material Name | Percentage (%) w/w |
|---|---|
| Regular Starch | 33 |
| Pre-gelatinized Starch | 4 |
| Glycerin | 12 |
| Chicken Liver Powder | 12 |
| Dextrose | 9.3 |
| Soy Oil | 9 |
| Lecithin | 3 |
| Praziquantel (Active) | <1 |
| Potassium Sorbate | <1 |
| Vitamin E 70 (Tocopherol) | <1 |
| Caramel Color liquid | 2 |
| Corn Syrup | 5 |
| Sorbitol | 5 |
| Crospovidone | 4 |
| Total Liquid | 33.1 |
| Total Starch Ratio (Regular Starch/Pre- | (33:4) |
| gelatinized Starch) | |

In this example, crospovidone disintegrant was used an the use of Regular Starch was reduced. It was observed that the dough was not formed and could not be extruded easily.

### Example 28—Soft Chewable Formulation

This example provides a soft chewable formulation of an embodiment of the disclosure as described in Table 28.

TABLE 28

| Raw Material Name | Percentage (%) w/w |
|---|---|
| Pre-gelatinized Starch | 44 |
| Glycerin | 14.5 |
| Chicken Liver Powder | 12 |
| Dextrose | 10.3 |
| Soy Oil | 8.5 |
| Lecithin | 3 |
| Praziquantel (Active) | <1 |
| Potassium Sorbate | <1 |
| Vitamin E 70 (Tocopherol) | <1 |
| Caramel Color liquid | 2.0 |
| Sorbitol | 6 |
| Total Liquid | 29.1 |
| Total Starch Ratio (Regular Starch/Pre- | N/A |
| gelatinized Starch) | |

In this example, only pre-gelatinized starch was used in the formulation. The dough was good, could be extruded, and the extruded chews provided desirable dissolution profile. However, the chews did not maintain desired softness and became hard in few weeks.

### Example 29—Soft Chewable Formulation

This example provides a soft chewable formulation of an embodiment of the disclosure as described in Table 29.

TABLE 29

| Raw Material Name | Percentage (%) w/w |
|---|---|
| Regular Starch | 21 |
| Pre-gelatinized Starch | 5 |
| Chicken Liver Powder | 10 |
| Dextrose | 11 |
| Lecithin Powder | 3 |
| Pyrantel Pamoate (Active) | 16.4 |
| Ivermectin (Active) | <1 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular Starch/Pre- | (21:5) |
| gelatinized Starch) | |

Dough was soft and could extrude easily.

### Example 30—Soft Chewable Formulation

This example provides a soft chewable formulation of an embodiment of the disclosure as described in Table 30.

TABLE 30

| Raw Material Name | Percentage (%) w/w |
|---|---|
| Regular Starch | 21 |
| Pre-gelatinized Starch | 5 |
| Chicken Liver Powder | 10 |
| Dextrose | 14 |
| Pyrantel Pamoate (Active) | 16.4 |
| Ivermectin (Active) | <1 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular Starch/Pre- | (21:5) |
| gelatinized Starch) | |

Dough was on the softer side and could extrude easily.

### Example 31—Soft Chewable Formulation

This example provides a soft chewable formulation of an embodiment of the disclosure as described in Table 31.

TABLE 31

| Raw Material Name | Percentage (%) w/w |
|---|---|
| Regular Starch | 24 |
| Pre-gelatinized Starch | 5 |
| Chicken Liver Powder | 10 |
| Dextrose | 11 |
| Pyrantel Pamoate (Active) | 16.4 |
| Ivermectin (Active) | <1 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular Starch/Pre- | (24:5) |
| gelatinized Starch) | |

Dough was on the softer side and could extrude easily.

### Example 32—Soft Chewable Formulation

This example provides a soft chewable formulation of an embodiment of the disclosure as described in Table 32.

TABLE 32

| Raw Material Name | Percentage (%) w/w |
|---|---|
| Regular Starch | 26 |
| Pre-gelatinized Starch | 5 |
| Chicken Liver Powder | 10 |
| Dextrose | 14 |
| Lecithin Powder | 3 |
| Pyrantel Pamoate (Active) | 8.2 |
| Ivermectin (Active) | <1 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular Starch/Pre- | (26:5) |
| gelatinized Starch) | |

Dough was on the softer side and could extrude easily.

### Example 33—Soft Chewable Formulation

This example provides a soft chewable formulation of an embodiment of the disclosure as described in Table 33.

TABLE 33

| Raw Material Name | Percentage (%) w/w |
|---|---|
| Regular Starch | 29 |
| Pre-gelatinized Starch | 5 |
| Chicken Liver Powder | 10 |
| Dextrose | 14 |
| Pyrantel Pamoate (Active) | 8.2 |
| Ivermectin (Active) | <1 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular Starch/Pre- | (29:5) |
| gelatinized Starch) | |

Doug was on the softer side an could extrude easily.

### Example 34—Soft Chewable Formulation

This example provides a soft chewable formulation of an embodiment of the disclosure as described in Table 34.

TABLE 34

| Raw Material Name | Percentage (%) w/w |
|---|---|
| Regular Starch | 32 |
| Pre-gelatinized Starch | 5 |
| Chicken Liver Powder | 10 |
| Dextrose | 11 |
| Pyrantel Pamoate (Active) | 8.2 |
| Ivermectin (Active) | <1 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular Starch/Pre- | (32:5) |
| gelatinized Starch) | |

Dough was soft and could extrude easily.

### Example 35—Soft Chewable Formulation

This example provides a soft chewable formulation of an embodiment of the disclosure as described in Table 35.

TABLE 35

| Raw Material Name | Percentage (%) w/w |
|---|---|
| Regular Starch | 34 |
| Pre-gelatinized Starch | 5 |
| Chicken Liver Powder | 10 |
| Dextrose | 11 |
| Lecithin Powder | 3 |
| Pyrantel Pamoate (Active) | 4.1 |
| Ivermectin (Active) | <1 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular Starch/Pre- | (34:5) |
| gelatinized Starch) | |

Dough would appear to be soft and could be extruded easily.

### Example 36—Soft Chewable Formulation

This example provides a soft chewable formulation of an embodiment of the disclosure as described in Table 36.

TABLE 36

| Raw Material Name | Percentage (%) w/w |
|---|---|
| Regular Starch | 37 |
| Pre-gelatinized Starch | 5 |
| Chicken Liver Powder | 10 |
| Dextrose | 11 |
| Pyrantel Pamoate (Active) | 4.1 |
| Ivermectin (Active) | <1 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular Starch/Pre- | (37:5) |
| gelatinized Starch) | |

It was observed that the dough was lightly firm but easy to extrude.

### Example 37—Soft Chewable Formulation

This example provides a soft chewable formulation of an embodiment of the disclosure as described in Table 37.

TABLE 37

| Raw Material Name | Percentage (%) w/w |
|---|---|
| Regular Starch | 31 |
| Pre-gelatinized Starch | 5 |
| Chicken Liver Powder | 10 |
| Dextrose | 14 |
| Lecithin Powder | 3 |
| Pyrantel Pamoate (Active) | 4.1 |
| Ivermectin (Active) | <1 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular Starch/Pre- | (31:5) |
| gelatinized Starch) | |

It was observed that the dough was soft and easy to be extruded.

### Example 38—Soft Chewable Formulation

This example provides a soft chewable formulation of an embodiment of the disclosure as described in Table 38.

TABLE 38

| Raw Material Name | Percentage (%) w/w |
|---|---|
| Regular Starch | 44 |
| Pre-gelatinized Starch | 4 |
| Chicken Liver Powder | 10 |
| Dextrose | 14 |
| Pyrantel Pamoate (Active) | 4.1 |
| Ivermectin (Active) | <1 |
| Potassium Sorbate Powder | <1 |
| Vitamin E T-70 (Tocopherol) | <1 |
| Refined Soybean Oil | 6 |
| Glycerin | 9 |
| Caramel Color Liquid | 1 |
| Corn Syrup | 4 |
| Sorbitol | 3 |
| Total Liquid | 23.1 |
| Total Starch Ratio (Regular Starch/Pre- | (44:4) |
| gelatinized Starch) | |

It was observed that the dough was soft and easy to be extruded.

### Example 39—Soft Chewable Formulation

This example provides a soft chewable formulation of an embodiment of the disclosure as described in Table 39.

TABLE 39

| Raw Material Name | Percentage (%) w/w |
|---|---|
| Regular Starch | 41 |
| Pre-gelatinized Starch | 4 |
| Chicken Liver Powder | 10 |
| Dextrose | 14 |
| Lecithin Powder | 3 |
| Pyrantel Pamoate (Active) | 4.1 |
| Ivermectin (Active) | <1 |
| Potassium Sorbate Powder | <1 |
| Vitamin E T-70 (Tocopherol) | <1 |
| Refined Soybean Oil | 6 |
| Glycerin | 9 |
| Caramel Color Liquid | 1 |
| Corn Syrup | 4 |
| Sorbitol | 3 |
| Total Liquid | 23.1 |
| Total Starch Ratio (Regular Starch/Pre- | (41:4) |
| gelatinized Starch) | |

It was observed that the dough was soft and easy to be extruded.

### Example 40—Soft Chewable Formulation

This example provides a soft chewable formulation of an embodiment of the disclosure as described in Table 40.

TABLE 40

| Raw Material Name | Percentage (%) w/w |
|---|---|
| Regular Starch | 36 |
| Pre-gelatinized Starch | 5 |
| Chicken Liver Powder | 10 |
| Dextrose | 11 |
| Lecithin Powder | 3 |
| Carprofen (Active) | 2.5 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular Starch/Pre- | (36:5) |
| gelatinized Starch) | |

It was observed that the dough was soft and easy to be extruded.

### Example 41—Soft Chewable Formulation

This example provides a soft chewable formulation of an embodiment of the disclosure as described in Table 41.

TABLE 41

| Raw Material Name | Percentage (%) w/w |
|---|---|
| Regular Starch | 41 |
| Pre-gelatinized Starch | 5 |
| Chicken Liver Powder | 10 |
| Dextrose | 11 |
| Carprofen (Active) | 0.6 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular Starch/Pre- | (41:5) |
| gelatinized Starch) | |

It was observed that the dough was soft and easy to be extruded.

### Example 42—Soft Chewable Formulation

This example provides a soft chewable formulation of an embodiment of the disclosure as described in Table 42.

TABLE 42

| Raw Material Name | Percentage (%) w/w |
|---|---|
| Regular Starch | 38 |
| Pre-gelatinized Starch | 5 |
| Chicken Liver Powder | 10 |
| Dextrose | 11 |
| Firocoxib (Active) | 2.9 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular Starch/Pre- | (38:5) |
| gelatinized Starch) | |

It was observed that the dough was soft an easy to be extrude.

### Example 43—Soft Chewable Formulation

This example provides a soft chewable formulation of an embodiment of the disclosure as described in Table 43.

TABLE 43

| Raw Material Name | Percentage (%) w/w |
|---|---|
| Regular Starch | 38 |
| Pre-gelatinized Starch | 5 |
| Chicken Liver Powder | 10 |
| Dextrose | 11 |
| Firocoxib (Active) | 2.9 |
| Potassium Sorbate Powder | <1 |
| Vitamin E T-70 (Tocopherol) | <1 |
| Refined Soybean Oil | 8 |
| Glycerin | 11 |
| Caramel Color Liquid | 2 |
| Corn Syrup | 3 |
| Sorbitol | 4 |
| Crospovidone | 4 |
| Croscarmellose | <1 |
| Total Liquid | 28.1 |
| Total Starch Ratio (Regular Starch/Pre- | (38:5) |
| gelatinized Starch) | |

It was observed that the dough was soft and easy to be extruded.

### Example 44—Soft Chewable Formulation

This example provides a soft chewable formulation of an embodiment of the disclosure as described in Table 44.

TABLE 44

| Raw Material Name | Percentage (%) w/w |
|---|---|
| Regular Starch | 46 |
| Pre-gelatinized Starch | 5 |
| Chicken Liver Powder | 10 |
| Dextrose | 11 |
| Firocoxib (Active) | 2.9 |
| Potassium Sorbate Powder | <1 |
| Vitamin E T-70 (Tocopherol) | <1 |
| Refined Soybean Oil | 7 |
| Glycerin | 9 |
| Caramel Color Liquid | 2 |
| Corn Syrup | 4 |
| Sorbitol | 2 |
| Croscarmellose | 1 |
| Total Liquid | 23.6 |
| Total Starch Ratio (Regular Starch/Pre- | (46:5) |
| gelatinized Starch) | |

It was observed that the dough was soft and easy to be extruded.

### Example 45—Soft Chewable Formulation

This example provides a soft chewable formulation of an embodiment of the disclosure as described in Table 45.

TABLE 45

| Raw Material Name | Percentage (%) w/w |
|---|---|
| Regular Starch | 29 |
| Pre-gelatinized Starch | 5 |
| Chicken Liver Powder | 10 |
| Dextrose | 11 |
| Lecithin Powder | 0 |
| Firocoxib (Active) | 11 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular Starch/Pre- | (29:5) |
| gelatinized Starch) | |

It was observed that the dough was soft and easy to be extruded.

### Example 46—Soft Chewable Formulation

This example provides a soft chewable formulation of an embodiment of the disclosure as described in Table 46.

TABLE 46

| Raw Material Name | Percentage (%) w/w |
|---|---|
| Regular Starch | 40 |
| Pre-gelatinized Starch | 5 |
| Chicken Liver Powder | 10 |
| Dextrose | 11 |
| Firocoxib (Active) | 1.4 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular | (40:5) |
| Starch/Pre-gelatinized Starch) | |

It was observed that the dough was soft and easy to be extruded.

### Example 47—Soft Chewable Formulation

This example provides a soft chewable formulation of an embodiment of the disclosure as described in Table 47.

TABLE 47

| Raw Material Name | Percentage (%) w/w |
|---|---|
| Regular Starch | 34 |
| Pre-gelatinized Starch | 5 |
| Chicken Liver Powder | 10 |
| Dextrose | 11 |
| Lecithin Powder | 3 |
| Maropitant Citrate (Active) | 4 |
| Potassium Sorbate Powder | <1 |
| Vitamin E T-70 (Tocopherol) | <1 |
| Refined Soybean Oil | 7 |
| Glycerin | 11 |
| Caramel Color Liquid | 2 |
| Corn Syrup | 4 |
| Sorbitol | 4 |
| Crospovidone | 4 |
| Croscarmellose | <1 |
| Total Liquid | 28.1 |
| Total Starch Ratio (Regular | (34:5) |
| Starch/Pre-gelatinized Starch) | |

It was observed that the dough was soft and easy to be extruded.

### Example 48

In this example, data from a praziquantel soft chew stability was determined.

Dough from Example 11 was manufactured and cut to approximately 4.0 g of a soft chew that included a dose of 34 mg of praziquantel per chew. Testing of Example 11 for stability of physical parameters and active drug content occurred at time 0, and then at time points and conditions: 1 month, 3 months, 6 Months at ambient and accelerated conditions of 40° C./75% RH. Testing performed for physical parameters included: hardness (tablet breaking force), moisture content, active assay, and single point dissolution. The assay analytical results for Example 11 were obtained via HPLC methodology. Assay results are described in Table 48.

TABLE 48

| | Months | Months | | |
|---|---|---|---|---|
| | Ambient Conditions | 40° C./75% RH | | |
| Formulation | 6 M | 1 M | 3 M | 6 M |
| Example 11 | 113.0 | 107.7 | 105.5 | 105.5 |

The analytical assay data of Example 11 shows that praziquantel at accelerated stability conditions are stable for up to 6 months and projected stability of up to 36 months at 25° C. room temperature with 95% assay of the required praziquantel dose.

For the related substances of praziquantel finished product and stability, the related substances estimation was performed by performed by HPLC. Assay results are provided in Table 49.

TABLE 49

| | Months | Months | | |
|---|---|---|---|---|
| | Ambient Conditions | 40° C./75% RH | | |
| Formulation | 6 M | 1 M | 3 M | 6 M |
| Example 11 | 0.07% | 0.09% | 0.12% | 0.13% |

Individually, for all strengths “Known Related Compound” were either none detected or less than 0.2%. Similarly, “Unknown related substances≥0.2%” were none detected. At both conditions, results were consistent with the USP specifications for Praziquantel as the API.

For the dissolution of praziquantel finished product and stability analysis, as per the current USP, a 3× sink condition was performed. A dissolution medium consisting of 0.2% Sodium lauryl sulfate in 0.1 N HCl was used. This dissolution medium allowed 3× sink conditions. Table 50 shows the dissolution conditions and the percent dissolved are given in Table 51. Dissolution samples were analyzed using HPLC.

TABLE 50

| | |
|---|---|
| Apparatus | USP Apparatus 2 (paddle) |
| Dissolution Medium | 0.2% Sodium Lauryl Sulfate in 0.1N HCl |
| Media volume: | 900 mL |
| Rotation | 100 |
| Vessels | Agilent TruCenter, Clear glass, 1-L, |
| | Peak Vessel w/collar (Part Number:12-5125) |
| Sample preparation: | Filtered a portion of the solution through a 0.45 μm |
| | Nylon filter. |

TABLE 51

| | |
|---|---|
| Dissolution | Praziquantel % Dissolved of the label Claim |
| Profile 100 RPM | @ Months (M) under Ambient Conditions |
| Example 11 | 6 M |

| | | |
|---|---|---|
| 60 | min | 54% |
| 120 | min | 86% |

The hardness of the chews was performed using Brookfield CT-3 Texture Analyzer. The peak load results are described in Table 52.

TABLE 52
Peak load in Newtons (N)

| | 5 | >24 | 1 | 3 | 6 |
|---|---|---|---|---|---|
| | Month | Month | Month | Month | Month |
| Formulation | Ambient Conditions | | 40° C./75% RH | | |
| Example 11 | 34.55 | 36.57 | 37.45 | 45.12 | 43.39 |

Hardness of chews at ambient stability condition after 27 months is very much comparable to initial hardness of soft chews.

Loss on drying was performed using aeABAM™, PMB 163 moisture analyzers. The LOD results are described in Table 53.

TABLE 53
Loss on drying

| | 6 Month | 1 Month | 3 Month | 6 Month |
|---|---|---|---|---|
| Formulation | Ambient Conditions | 40° C./75% RH | | |
| Example 11 | 8.8 | 8.6 | 9.3 | 7.7 |

The results show the invention maintains 95% of original softness for a projected 18 years when held at 25° C., as determined by the Arrhenius equation for stability.

### Example 49

In this example, data from a firocoxib soft chew stability was determined.

Dough from Example 42 was manufactured and cut to approximately 2.0 g of a soft chew that included a dose of 57 mg of firocoxib per chew. Testing of Example 42 for stability of physical parameters and active drug content occurred at time 0, and then at time points and conditions: 3 months and 6 Months at ambient and accelerated conditions of 40° C./75% RH. Testing performed for physical parameters included: hardness (tablet breaking force), moisture content, active assay, and single point dissolution. The assay analytical results for Example 42 were obtained via HPLC methodology. Assay results are described in Table 54

TABLE 54

| % Assay @ Months | |
|---|---|
| Ambient Conditions | 40° C./75% RH |

| Formulation | 3 M | 9 M | 3 M | 6 M |
|---|---|---|---|---|
| Example 42 | 103.7 | 103.3 | 105.3 | 103.3 |

For the related substances of firocoxib finished product and stability, the related substances estimation was performed by performed by HPLC. Assay results are provided in Table 55.

TABLE 55

| % Related Substances @ Months | |
|---|---|
| Ambient Conditions | 40° C./75% RH |

| Formulation | 3 M | 9 M | 3 M | 6 M |
|---|---|---|---|---|
| Example 42 | <0.2% | <0.2% | <0.2% | <0.2% |

The analytical assay data of Example 42 shows that firocoxib formulation at accelerated stability conditions are stable for up to 6 months and projected stability of up to 24 months at 25° C. room temperature.

For the dissolution of firocoxib finished product and stability analysis, as per the current USP, a 3× sink condition was performed. A dissolution medium consisting of Sa cetrimide solution with a pH of 4.5. This dissolution medium allowed 3× sink conditions. Table 56 shows the dissolution conditions and the percent dissolved are given in Table 56. Dissolution samples were analyzed using HPLC.

TABLE 56

| | |
|---|---|
| Apparatus | USP Apparatus 2 (paddle) |
| Dissolution Medium | 1% Cetrimide Solution (pH = 4.5) |
| Media volume: | 900 mL |
| Rotation | 75 |
| Vessels | Clear glass, 1-L, Peak Vessel w/collar |
| Sample preparation: | Filtered a portion of the solution through a 0.22 μm |
| | PTFE filter. |

TABLE 57

| | |
|---|---|
| Dissolution | Firocoxib % Dissolved of the label Claim |
| Profile 75 RPM | @ Months (M) under Ambient Conditions |

| Example 43 | 3 M | 9 M |
|---|---|---|
| 30 min | 82% | 83% |
| 90 min | 99% | 96% |

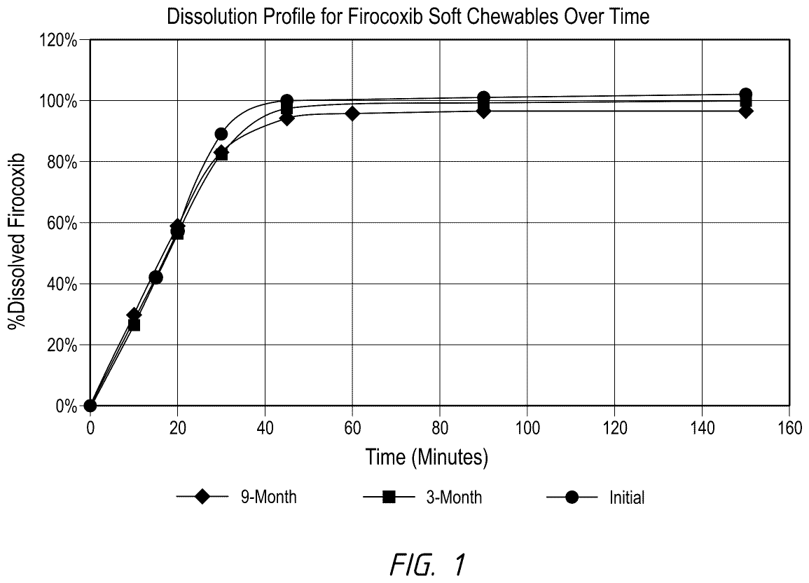

The dissolution profile from this example over a period of 9 months is shown in FIG. 1. The hardness of the chews was performed using Brookfield CT-3 Texture Analyzer. The peak load results are described in Table 47.

TABLE 58
Peak load in Newtons (N)

| | |
|---|---|
| 3 Month | 9 Month |

| | |
|---|---|
| Formulation | Ambient Conditions |

| | | |
|---|---|---|
| Example 42 | 14 | 9 |

Hardness of chews at ambient stability condition after 9 months is very much comparable to initial hardness of soft chews.

Loss on drying was performed using a moisture analysis method. The LOD results are described in Table 59.

TABLE 59
Loss on drying

| | |
|---|---|
| 3 Month | 9 Month |

| | |
|---|---|
| Formulation | Ambient Conditions |

| | | |
|---|---|---|
| Example 42 | 7.3% | 8.0% |

The results show the invention maintains 95% of original characteristics for a projected 2 years when held at 25° C., as determined by the Arrhenius equation for stability.

### Example 50

In this example, data from a firocoxib soft chew stability was determined.

Dough from Example 37 was manufactured and cut to approximately 2.0 g of a soft chew that included a dose of 57 mg of firocoxib per chew. Testing of Example 50 for stability of physical parameters and active drug content occurred at time 0, and then at time points and conditions: 3 months at ambient and accelerated conditions of 40° C./75% RH. Testing performed for physical parameters included: hardness (tablet breaking force), moisture content, active assay, and single point dissolution. The assay analytical results for Example 50 were obtained via HPLC methodology. Assay results are described in Table 60.

TABLE 60

% Assay @ Months

| | Ambient Conditions | 40° C./75% RH |
|---|---|---|
| Formulation | 3 M | 3 M |
| Example 37 | 99.1% | 97.5% |

For the related substances of firocoxib finished product and stability, the related substances estimation was performed by performed by HPLC. Assay results are provided in Table 61.

TABLE 61

% Related Substances @ Months

| | Ambient Conditions | 40° C./75% RH |
|---|---|---|
| Formulation | 3 M | 3 M |
| Example 37 | <0.2% | <0.2% |

For the dissolution of firocoxib finished product and stability analysis, as per the current USP, a 3× sink condition was performed. A dissolution medium consisting of 1% cetrimide solution with a pH of 4.5. This dissolution medium allowed 3× sink conditions. Table 62 shows the dissolution conditions and the percent dissolved are given in Table 63. Dissolution samples were analyzed using HPLC.

TABLE 62

| | |
|---|---|
| Apparatus | USP Apparatus 2 (paddle) |
| Dissolution Medium | 1% Cetrimide Solution (pH = 4.5) @ 37° C. |
| Media volume: | 900 mL |
| Rotation | 75 |
| Vessels | Clear glass, 1-L, Peak Vessel w/collar |
| Sample preparation: | Filtered a portion of the solution through a 0.22 μm |
| | PTFE filter. |

TABLE 63

| Dissolution | Firocoxib % Dissolved of the label Claim @ |
|---|---|
| Profile 75 RPM | Months (M) under Ambient Conditions |
| Example 37 | 3 M |
| 30 min | 69% |
| 90 min | 92% |

The hardness of the chews was performed using Brookfield CT-3 Texture Analyzer. The peak load results are described in Table 64.

TABLE 64
Peak load in Newtons (N)

| | 3 Month |
|---|---|
| Formulation | Ambient Conditions |
| Example 37 | 13 |

Loss on drying was performed using a moisture analysis method. The LOD results are described in Table 65.

TABLE 65
Loss on drying

| | 3 Month |
|---|---|
| Formulation | Ambient Conditions |
| Example 37 | 7.0% |

The results show the soft chewables in Example 37 provide the desired properties for a medicated soft chewable and is likely to remain stable over a 2-year shelf period.

### Example 51

In this example, data from a Ivermectin/Pyrantel Pamoate soft chew stability was determined.

Dough from Example 36 was manufactured and cut to approximately 4.0 g of a soft chew that included a dose of 68 microgram of ivermectin and 57 mg of pyrantel as pamoate salt per chew. Testing of Example 36 for stability of physical parameters and active drug content occurred at time 0, and then at 9 Months at ambient condition. Testing performed for physical parameters included: hardness (tablet breaking force), moisture content, active assay, and single point dissolution. The assay analytical results for Example 36 were obtained via HPLC methodology. Assay results are described in Table 66.

TABLE 66

| | |
|---|---|
| Formulation | Months |
| Example 36 | Ambient |

| Assay (% Label) | Initial | 9 M |
|---|---|---|
| Ivermectin | 105% | 97% |
| Pyrantel Pamoate | 97% | 95% |

No reportable degradation products (0.2) were observed either initially or at 9M of ambient storage. The analytical assay data of Example 51 show that Ivermectin and Pyrantel Pamoate in the soft chewable example are likely to stay stable over a 2-year shelf life.

For the dissolution of pyrantel pamoate in finished product and stability analysis, as per the current USP, a 3× sink condition was performed. A dissolution medium consisting of 1% SLS (pH=7.0). This dissolution medium allowed 3× sink conditions. Table 67 shows the dissolution conditions and the percent dissolved are given in Table 68. Dissolution samples were analyzed using HPLC.

TABLE 67

| | |
|---|---|
| Apparatus | USP Apparatus 2 (paddle) |
| Dissolution Medium | 1% SLS (pH = 7.0) @ 37° C. |
| Media volume: | 900 mL |
| Rotation | 75 |
| Vessels | Clear glass, 1-L, Peak Vessel w/collar |
| Sample preparation: | Filtered a portion of the solution through a 0.45 μm |
| | Nylon filter. |

TABLE 68

| | |
|---|---|
| Dissolution | Pyrantel Pamoate % Dissolved of the label Claim |
| Profile 75 RPM | @ Months (M) under Ambient Conditions |
| Example 36 | 9 M |

| | | |
|---|---|---|
| 60 | min | 93% |
| 120 | min | 97% |

The hardness of the chews was performed using Brookfield CT-3 Texture Analyzer. The peak load results are described in Table 69.

TABLE 69
Peak load in Newtons (N)

| | 9 Months |
|---|---|
| Formulation | Ambient Conditions |
| Example 36 | 17.8 |

Loss on drying was performed using forced air convection oven at 60° C. The LOD results are described in Table 48.

TABLE 70
Loss on Drying (%)

| | 9 Months |
|---|---|
| Formulation | Ambient Conditions |
| Example 36 | 8.6% |

The results show the soft chewables in Example 36 provide the desired properties for a medicated soft chewable and is likely to remain stable over a 2-year shelf period.

### Example 52

In this example, data from a Ivermectin/Pyrantel Pamoate soft chew stability was determined.

Dough from Example 37 was manufactured and cut to approximately 4.0 g of a soft chew that included a dose of 68 microgram of ivermectin and 57 mg of pyrantel as pamoate salt per chew. Testing of Example 36 for stability of physical parameters and active drug content occurred at time 0, and then at 9 Months at ambient condition. Testing performed for physical parameters included: hardness (tablet breaking force), moisture content, active assay, and single point dissolution. The assay analytical results for Example 36 were obtained via HPLC methodology. Assay results are described in Table 71.

TABLE 71

| | |
|---|---|
| Formulation | Months |
| Example 37 | Ambient |

| Assay (% Label) | Initial | 9 M |
|---|---|---|
| Ivermectin | 99% | 98% |
| Pyrantel Pamoate | 99% | 98% |

No reportable degradation products (0.2) were observed either initially or at 9M of ambient storage. The analytical assay data of Example 37 show that Ivermectin and Pyrantel Pamoate in the soft chewable example are likely to stay stable over a 2-year shelf life.

For the dissolution of pyrantel pamoate in finished product and stability analysis, as per the current USP, a 3× sink condition was performed. A dissolution medium consisting of 1% SLS (pH=7.0). This dissolution medium allowed 3× sink conditions. Table 72 shows the dissolution conditions and the percent dissolved are given in Table 73. Dissolution samples were analyzed using HPLC.

TABLE 72

| | |
|---|---|
| Apparatus | USP Apparatus 2 (paddle) |
| Dissolution Medium | 1% SLS (pH = 7.0) @ 37° C. |
| Media volume: | 900 mL |
| Rotation | 75 |
| Vessels | Clear glass, 1-L, Peak Vessel w/collar |
| Sample preparation: | Filtered a portion of the solution through a 0.45 μm |
| | Nylon filter. |

TABLE 73

| | |
|---|---|
| Dissolution | Pyrantel Pamoate % Dissolved of the label Claim |
| Profile 75 RPM | @ Months (M) under Ambient Conditions |
| Example 37 | 9 M |

| | | |
|---|---|---|
| 60 | min | 62% |
| 120 | min | 105% |

The hardness of the chews was performed using Brookfield CT-3 Texture Analyzer. The peak load results are described in Table 73.

TABLE 73
Peak load in Newtons (N)

| | 9 Months |
|---|---|
| Formulation | Ambient Conditions |
| Example 37 | 28.0 |

Loss on drying was performed using forced air convection oven at 60° C. The LOD results are described in Table 74.

TABLE 74
Loss on Drying (%)

| | 9 Months |
|---|---|
| Formulation | Ambient Conditions |
| Example 37 | 6.9% |

The results show the soft chewables in Example 37 provide the desired properties for a medicated soft chewable and is likely to remain stable over a 2-year shelf period.

It is concluded that the stability of various soft chew formulations produced under this disclosure and related physical criteria is stable over the duration of study and meets regulatory acceptance criteria.

### CONCLUSION

In this example, it was discovered specified ratios of starch to pregelatinized starch with no water added to the process affect physical parameters of soft chew formats when combined in particular with active pharmaceutical drugs and excipients in the absence of water and heat. Unexpectedly, it was discovered that when starch is combined with pregelatinized starches in certain ratios from ranging from 4:1 to 11:1, the swelling capability is affected in a positive manner that allows for actives and other excipients to have optimal physical final matrix characteristics that allow for extrudability that is not too dry or too sticky. It should be noted that the combination of certain excipients in combination with actives can cause problems of texture and density of soft chew formulations. However, these novel standard starch to pregelatinized starch ratios allow for the matrix to remain pliable with a specified degree of hardness that is sufficient to retain its shape under normal manufacturing, shipping, storage and handling conditions over the course of a shelf-life of two years. In addition, the starch to pregelatinized starch ratios allow for stability of the actives, dissolution of the matrix and palatability to meet the specifications of a shelf-life of a minimum of two years. With these novel features, the current disclosure of soft chew formulations is particularly suitable for moisture-sensitive drugs. In accordance with one aspect of the present invention, there is provided a soft chew that includes one or more pharmaceutically-active or nutritionally-active ingredients and a plurality of excipients.

Many soft chewable formulations intended for veterinary or human administration use a similar formulation approach with ingredients consisting of diluents, flavor, compressible sugars, emulsifying agents, texturizers, flow aid, lubricants, binders, animal or vegetable-sourced proteins, disintegrants, pigments, water, etc. However, many of these formulations suffer from stability issues of the actives and the matrix itself for maintaining palatability, softness, and dissolution characteristics over the defined shelf life. This is due to the interaction of drugs and specific ingredients and concentrations of these ingredients in the soft chew matrix. Hence, a new soft chewable formulation that can meet the regulatory requirements for dissolution in physiologically relevant media is needed. This has been a challenge for a number of drug actives in soft chew formulations. The ability to extrude any particular formulation that is not too dry or too sticky for the equipment used continues to be a challenge to manufacturers. Furthermore, maintaining the stability of many active drugs is a challenge in soft chew formats because the presence of water is typically determinantal to drug actives.

In some embodiments, the inventors have overcome a number of these issues in the current invention with the use of certain ratios of regular starch to pregelatinized starch to maintain stability in a soft chew formulation. The use of starches in this example appear to play the functional role as a thickener, binding agent, emulsifier, and gelling agent. Starch will swell when heated in water and take on a gelatin like physical appearance. Starch gelatinization is a process of breaking down the intermolecular bonds of starch molecules in the presence of water and heat, allowing the hydrogen bonding sites (the hydroxyl hydrogen and oxygen) to engage more water. This irreversibly dissolves the starch granule in water. Water acts as a plasticizer. Pregelatinized starch is starch which has been cooked and then dried into a powder making the pregelatinized starch powder to be cold-water-soluble. Pre-gelatinized starches are plant-based ingredients typically made from arrowroot, potato, wheat, corn or tapioca. This means it is entirely suitable for vegetarian and vegan consumption. Pre-gelatinized starch dissolves in cold liquids and becomes viscous. Because pregelatinized starch easily dissolves in cold liquids, it eliminates a heating step in manufacturing and allows for the incorporation of drug or nutraceutical actives that are sensitive to heat and water to maintain stability.

Thus, in some embodiments, the inventors have discovered surprisingly and unexpectantly that certain ratios of normal starch to pregelatinized starch can play a significant role in extrudability, maintaining the stability of the actives and the chew matrix for palatability, softness and disintegration over the shelf life of the finished product.

1. A soft chewable veterinary formulation, the formulation comprising:
a starch between about 2% w/w and about 50% w/w;
one or more carbohydrates between about 5% w/w and about 25% w/w;
a flavoring agent between about 5% w/w and about 15% w/w;
a disintegrant between about 0.1% w/w and about 10.0% w/w;
a lipid between about 5% w/w and about 15% w/w; and
at least one active ingredient between about 0.001% w/w and about 40% w/w,
wherein, the starch comprises a regular starch and a pre-gelatinized starch that is in a ratio from about 3:1 to about 15:1.

2. The soft chewable veterinary formulation of claim 1, wherein the flavoring agent is an animal-derived flavoring agent.

3. The soft chewable veterinary formulation of claim 2, wherein the animal-derived flavoring agent is chicken liver powder, pork liver powder, beef liver powder, ham, fish, or a combination thereof.

4. The soft chewable veterinary formulation of claim 3, wherein the chicken-liver comprises about 8% to about 14% of the soft chewable veterinary formulation.

5. The soft chewable veterinary formulation of claim 1, wherein the soft chewable veterinary formulation further comprises a humectant.

6. The soft chewable veterinary formulation of claim 5, wherein the humectant is glycerin, glycerol triacetate, polydextrose, lactic acid, or a combination thereof.

7. The soft chewable veterinary formulation of claim 5, wherein the humectant comprises between about 8% w/w and about 20% w/w of the soft chewable veterinary formulation.

8. The soft chewable veterinary formulation of claim 1, wherein the soft chewable veterinary formulation further comprises a preservative.

9. The soft chewable veterinary formulation of claim 8, wherein the preservative is benzalkonium chloride, benzethonium chloride, benzoic acid, benzyl alcohol, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, propyl paraben, myristyl gamma-picolinium chloride, methylparaben, propylparaben, quaternary ammonium compounds, or combinations thereof.

10. The soft chewable veterinary formulation of claim 8, wherein the preservative comprises between about 0.05% w/w to about 3.0% w/w of the soft chewable veterinary formulation.

11. The soft chewable veterinary formulation of claim 1, wherein the soft chewable veterinary formulation further comprises a binder.

12. (canceled)

13. The soft chewable veterinary formulation of claim 1, wherein the disintegrant is sodium starch glycolate, crospovidone, croscarmellose sodium, microcrystalline cellulose, alginic acid, veegum, bentonite, croscarmellose, or combinations thereof.

14. (canceled)

15. The soft chewable veterinary formulation of claim 1, wherein the soft chewable veterinary formulation further comprises an emulsifying agent.

16. (canceled)

17. (canceled)

18. The soft chewable veterinary formulation of claim 1, wherein the at least one active ingredient is a moisture-sensitive active ingredient.

19. The soft chewable veterinary formulation of claim 1, wherein the at least one active ingredient is at least one active anti-parasitic active ingredient.

20. The soft chewable veterinary formulation of claim 1, wherein the at least one active ingredient is selected from the group consisting of pyrantel pamoate, ivermectin, pimobendan, firocoxib, carprofen, maropitant citrate, and praziquantel.

21. (canceled)

22. (canceled)

23. (canceled)

24. (canceled)

25. (canceled)

26. (canceled)

27. (canceled)

28. (canceled)

29. (canceled)

30. (canceled)

31. The soft chewable veterinary formulation of claim 1, wherein the chewable veterinary formulation further comprises one or more components selected from the group consisting of surfactants, wetting agents, pH stabilizers, a softening agent, and a solvent.

32. (canceled)

33. (canceled)

34. (canceled)

35. (canceled)

36. A method of treating, controlling, preventing, or ameliorating a disease or condition in a subject in need thereof, the method comprising:
administering to the subject a soft chewable formulation of claim 1,
thereby, treating, controlling, preventing, or ameliorating the disease or condition in the subject in need thereof.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. The method of claim 36, wherein the disease or condition is inflammation or pain.

50. The method of claim 36, wherein administering to the soft chewable formulation is administered to the subject to treat, prevent, control, delay the onset of, reduce the symptoms of, or ameliorate the pain in the subject.
